SEQUENCE LISTING (19 sequences)

SEQ ID NO: 1 - DNA, 1751 bp, Homo sapiens
ctggaccttccacaagtgtttgcacatgacctggaagacagtcagaggcagaagtccctgcaaatgctcctggctacttcctgcaggcagctcatgctgctgcagcagaagactttcctatgtctagctgccagtattctgggtgctgggggcatcctgttcctaggatcacagccatggtggtatgttctagaactcagaaatttaccaaatcactggatggaccagaggagtgagctccaggttgggtttccagaagggactcccagaactcttccaccatacaggcctctcagggagctgctccctctgccatggtcagtgagagggggaagcaggagccgccattggggttgttgagttcgtggctgcaacccagggatgaggaagctgctgctacacacccatgaagctgatgcctggacataaatccctactgataagtgtttacgacatttccagcgtggtgccgacactgcatggaaatgctgcatggaaagtccttatacatctatctttgtgcatttattgtgagcacctactatgagtaaaacctgggctggtggctggagaaacatgaagatgagtaagagccaattcctgttcttggggatttaataatatattcaagggaaaagacacaaaataaccatttccaggtaaactctggtggggagggtgggaggagaaccgtggtttgcttttgtgcccaacacttcacattccttacctctttctccccaccgagaccttgaggagcagcctgagccagaggaccagcatcctcatcttctctccacctcttactaggggggtggctttgggccagtttcttacattctttgggccactttagtttccttattgaaaaatggggataataatagtggctacatctcagtgtgattctgaggaaaccagaattgtacatgcagagcacatgcacagaacagtgcctggcacagtcagtaattgatcaatgtgcgctattgttgttgtgtatattagttctcccctgatacagatgagagctatctctaactcagagacttgccacatcattaaattaggagtgagaactgagcctggataaggaagaggaacaggaattcaacacagtgaacacagcaggaaaggcttgtgcgaaggctctgggctggagatgtgtaaagcatgtctggagatggggaaggtccattgggccaggaaaccacaggctctgctctcctggagcagaagcacagcgaaatcacagccgagcagaggagggcaagggagagggccgccccagtctgagagctgcagggtctggctggaactgctcccggccagcggacttcacccgggcgcgggggccgcacctgccgggcgcggcctgctctatggcgccctctgctgttagtccgccccaggctccgcgccggcctctcctgggtccgtggggcctgcgggctgcggggatcaccgagacccacattcccgtggccagcagcctttcgctctgctcagaggagaggcagaagggcatattgctgtttcccagtcgctttttacacctgccttcttcggataaacccaaaaatcttccttcagagaagacggcccgtatttcccgttatttgggggtggaggtggggctaagggcgtcatagggagagccttactttcaacattctgcattatgaaaccaagggagactttttttcccaacaagtgtgaacattttttttcaagagaattaaatcgtttat

SEQ ID NO: 2 - RNA, 27 nt, Homo sapiens
agggagcugcucccucugccaugguca

SEQ ID NO: 3 - RNA, 27 nt, Homo sapiens
cagcaggaaaggcuugugcgaaggcuc

SEQ ID NO: 4 - RNA, 27 nt, Artificial Sequence
  Synthetic
ugaccauggcagagggagcagcucccu

SEQ ID NO: 5 - RNA, 27 nt, Artificial Sequence
  Synthetic
agggagcugcucccucugccaugguca

SEQ ID NO: 6 - RNA, 27 nt, Artificial Sequence
  Synthetic
gagccuucgcacaagccuuuccugcug

SEQ ID NO: 7 - RNA, 27 nt, Artificial Sequence
  Synthetic
cagcaggaaaggcuugugcgaaggcuc

SEQ ID NO: 8 - RNA, 1751 nt, Homo sapiens
cuggaccuuccacaaguguuugcacaugaccuggaagacagucagaggcagaagucccugcaaaugcuccuggcuacuuccugcaggcagcucaugcugcugcagcagaagacuuuccuaugucuagcugccaguauucugggugcugggggcauccuguuccuaggaucacagccauggugguauguucuagaacucagaaauuuaccaaaucacuggauggaccagaggagugagcuccagguuggguuuccagaagggacucccagaacucuuccaccauacaggccucucagggagcugcucccucugccauggucagugagagggggaagcaggagccgccauugggguuguugaguucguggcugcaacccagggaugaggaagcugcugcuacacacccaugaagcugaugccuggacauaaaucccuacugauaaguguuuacgacauuuccagcguggugccgacacugcauggaaaugcugcauggaaaguccuuauacaucuaucuuugugcauuuauugugagcaccuacuaugaguaaaaccugggcugguggcuggagaaacaugaagaugaguaagagccaauuccuguucuuggggauuuaauaauauauucaagggaaaagacacaaaauaaccauuuccagguaaacucugguggggagggugggaggagaaccgugguuugcuuuugugcccaacacuucacauuccuuaccucuuucuccccaccgagaccuugaggagcagccugagccagaggaccagcauccucaucuucucuccaccucuuacuagggggguggcuuugggccaguuucuuacauucuuugggccacuuuaguuuccuuauugaaaaauggggauaauaauaguggcuacaucucagugugauucugaggaaaccagaauuguacaugcagagcacaugcacagaacagugccuggcacagucaguaauugaucaaugugcgcuauuguuguuguguauauuaguucuccccugauacagaugagagcuaucucuaacucagagacuugccacaucauuaaauuaggagugagaacugagccuggauaaggaagaggaacaggaauucaacacagugaacacagcaggaaaggcuugugcgaaggcucugggcuggagauguguaaagcaugucuggagauggggaagguccauugggccaggaaaccacaggcucugcucuccuggagcagaagcacagcgaaaucacagccgagcagaggagggcaagggagagggccgccccagucugagagcugcagggucuggcuggaacugcucccggccagcggacuucacccgggcgcgggggccgcaccugccgggcgcggccugcucuauggcgcccucugcuguuaguccgccccaggcuccgcgccggccucuccuggguccguggggccugcgggcugcggggaucaccgagacccacauucccguggccagcagccuuucgcucugcucagaggagaggcagaagggcauauugcuguuucccagucgcuuuuuacaccugccuucuucggauaaacccaaaaaucuuccuucagagaagacggcccguauuucccguuauuuggggguggagguggggcuaagggcgucauagggagagccuuacuuucaacauucugcauuaugaaaccaagggagacuuuuuuucccaacaagugugaacauuuuuuuucaagagaauuaaaucguuuau

SEQ ID NO: 9 - DNA, 21 bp, Homo sapiens
cagcagcttcctcatccctgg

SEQ ID NO: 10 - DNA, 21 bp, Homo sapiens
tccccctctcactgaccatgg

SEQ ID NO: 11 - DNA, 21 bp, Homo sapiens
cctgagaggcctgtatggtgg

SEQ ID NO: 12 - RNA, 25 nt, Artificial Sequence
  Synthetic
cuuccucucuuucucucccuuguga

SEQ ID NO: 13 - RNA, 27 nt, Artificial Sequence
  Synthetic
cuuccucucuuucucucccuugccccc

SEQ ID NO: 14 - RNA, 27 nt, Artificial Sequence
  Synthetic
uccuuccucucuuucucucccuuguga

SEQ ID NO: 15 - DNA, 21 bp, Artificial Sequence
  Synthetic
gcagcuucauaaccgaagatt

SEQ ID NO: 16 - DNA, 20 bp, Artificial Sequence
  Synthetic
ucuucgguuaugagcugctt

SEQ ID NO: 17 - DNA, 21 bp, Artificial Sequence
  Synthetic
gguugaagcuuacuccggatt

SEQ ID NO: 18 - RNA, 21 nt, Artificial Sequence
  Synthetic
uccggaguaagcuucaaccac

SEQ ID NO: 19 - DNA, 27 bp, Artificial Sequence
  Synthetic
tccuuccucucuuucucucccuuguga

(19) United States
(12) Patent Application Publication
(10) Pub. No.: US 2016/0220599 A1
(43) Pub. Date: Aug. 4, 2016

(54) NOVEL LINCRNA AND INTERFERING NUCLEIC ACID MOLECULES, COMPOSITIONS AND METHODS AND USES THEREOF FOR REGULATING ANGIOGENESIS AND RELATED CONDITIONS

(71) Applicants: ST. MICHAEL'S HOSPITAL, Toronto (CA); THE HOSPITAL FOR SICK CHILDREN, Toronto (CA)

(72) Inventors: Philip A. Marsden, Toronto (CA); Sunit Das, Toronto (CA); Jenny Jing Wang, Toronto (CA); Megan Yijun Wu, Toronto (CA)

(21) Appl. No.: 15/024,406

(22) PCT Filed: Sep. 26, 2014
(86) PCT No.: PCT/CA2014/050930

§ 371 Date: Mar. 24, 2016

Related U.S. Application Data
(60) Provisional application No. 61/883,643, filed on Sep. 27, 2013.

(51) Int. Cl. A61K 31/7105; A61K 45/06; C12N 15/113

(52) U.S. Cl. CPC A61K 31/7105; C12N 15/113; A61K 45/06; C12N 2310/11; C12N 2310/14

(57) ABSTRACT

The present disclosure provides a large intergenic RNA (LIVE) and compositions thereof. Also provided is a nucleic acid molecule that silences the expression of the LIVE. Further provided are methods and uses of the compositions for modulating angiogenesis.

### CROSS-REFERENCE TO RELATED APPLICATIONS

This application claims the benefit of U.S. Provisional Application No. 61/883,643, filed Sep. 27, 2013, which is herein incorporated by reference in its entirety.

### FIELD

The present disclosure relates to long intergenic non-coding RNAs that are VEGF responsive and to interfering RNAs thereof. The disclosure also relates to compositions, methods and uses for regulating angiogenesis.

### BACKGROUND

The functional relevance of the mammalian transcriptome extends beyond its role as the messenger between the genome and proteome. The genome is populated by thousands of genes encoding long non-protein-coding transcripts (>200 bp in length), termed long noncoding RNAs (lncRNAs), which include natural anti-sense transcripts (NATs), pseudogenes, and large intergenic non-coding RNAs (lincRNAs). Although previously viewed as “dark matter”, lncRNAs have recently emerged as critical effector molecules in various cellular processes, including regulation of chromatin states, transcription, RNA stability, alternative splicing, translational efficiency, and cellular signaling (Kino, Hurt et al. 2010; Orom, Derrien et al. 2010; Tripathi, Ellis et al. 2010; Cesana, Cacchiarelli et al. 2011; Gong and Maquat 2011; Guttman, Donaghey et al. 2011; Ulitsky, Shkumatava et al. 2011).

Furthermore, recent reports have shown that dysregulation of lncRNA transcript levels is associated with human disease (Roberts, Abraira et al. 2012; Yang, U et al. 2012; Zhang, Chen et al. 2012), and have been implicated in cancer biology (Gupta, Shah et al. 2010; Kotake, Nakagawa et al. 2011; Lai, Yang et al. 2011; Prensner, lyer et al. 2011; Ren, Peng et al. 2012; Yang, Li et al. 2012). For example, the lincRNA HOTAIR has been found to be expressed at increased levels in primary breast tumours and to confer an invasive phenotype in a human breast carcinoma xenograft model (Gupta, Shah et al. 2010). While the role of lncRNAs in development and the maintenance of the stem cell state have been established, their functional impact, and that of lincRNAs in particular, on physiological processes outside of development has not been broadly studied (Pandey, Mondal et al. 2008; Cesana, Cacchiarelli et al. 2011; Ulitsky, Shkumatava et al. 2011; Wang, Yang et al. 2011).

Endothelial cells line every blood and lymphatic vessel throughout the human body. Angiogenesis and the maintenance of vascular health are largely dependent on the endothelial monolayer. Although natural antisense transcripts have been functionally implicated in vascular function and angiogenesis, the role of lincRNAs in endothelial biology remains unknown (Robb, Carson et al. 2004; Fish, Matouk et al. 2007; Li, Blum et al. 2010). Endothelial cells must be able to adopt different structural morphologies depending on the surrounding milieu, whether in a macrovascular context, such as the aorta, or in a microvascular network, such as capillary beds. Moreover, it is important for endothelial cells to respond to extracellular factors, such as vascular endothelial growth factor (VEGF), which has a crucial role in endothelial vascular physiology and angiogenesis (Ferrara, Gerber et al. 2003; Spoerke, Murray et al. 2005; Leimgruber, Ostermann et al. 2006; Dutta, Ray et al. 2008).

### SUMMARY

As lncRNAs directly participate in various cellular processes, the present inventors have demonstrated that the lncRNA fraction of the transcriptome of the vascular endothelium is dynamically regulated to a similar extent as the proteome in response to extracellular signals and morphological changes. The present inventors have shown that lncRNAs, and more specifically lincRNAs, are differentially expressed in response to VEGF-A and hold functional relevance to endothelial biology. These endothelial VEGF-A-responsive lincRNAs were examined for functional effects on vascular biology and contribution to the pathobiology of human disease, specifically in the context of tumour-associated blood vessel formation in glioblastoma multiforme (GBM).

Accordingly, the present disclosure provides an isolated nucleic acid encoding a large intergenic RNA (LIVE), wherein the nucleic acid comprises the nucleic acid sequence as shown in SEQ ID NO:1 or a variant thereof. Also provided is a vector comprising the nucleic acid encoding LIVE RNA or variant thereof and a host cell transformed with the vector.

In another aspect, there is provided a method of promoting angiogenesis in a cell or animal in need thereof comprising administering to the cell or animal in need thereof the nucleic acid encoding the LIVE RNA or variant thereof, the vector comprising the nucleic acid or the host cell transformed with the vector. Also provided is a use of the nucleic acid encoding the LIVE RNA or variant thereof, the vector comprising the nucleic acid or the host cell transformed with the vector for promoting angiogenesis in a cell or animal in need thereof. Further provided is use of the nucleic acid encoding the LIVE RNA or variant thereof, the vector comprising the nucleic acid or the host cell transformed with the vector in the manufacture of a medicament for promoting angiogenesis in a cell or animal in need thereof. Even further provided is the nucleic acid encoding the LIVE RNA or variant thereof, the vector comprising the nucleic acid or the host cell transformed with the vector for use in promoting angiogenesis in a cell or animal in need thereof.

In an embodiment, the methods and uses for promoting angiogenesis are useful in treating ischemic heart disease, peripheral vascular disease, cerebrovascular disease or preeclampsia. Accordingly, also provided herein is a method of treating ischemic heart disease, peripheral vascular disease, cerebrovascular disease or preeclampsia comprising administering to the cell or animal in need thereof the nucleic acid encoding the LIVE RNA or variant thereof, the vector comprising the nucleic acid or the host cell transformed with the vector. Also provided is a use of the nucleic acid encoding the LIVE RNA or variant thereof, the vector comprising the nucleic acid or the host cell transformed with the vector for treating ischemic heart disease, peripheral vascular disease, cerebrovascular disease or preedampsia in a cell or animal in need thereof. Further provided is use of the nucleic acid encoding the LIVE RNA or variant thereof, the vector comprising the nucleic acid or the host cell transformed with the vector in the manufacture of a medicament for treating ischemic heart disease, peripheral vascular disease, cerebrovascular disease or preeclampsia in a cell or animal in need thereof. Even further provided is the nucleic acid encoding the LIVE RNA or variant thereof, the vector comprising the nucleic acid or the host cell transformed with the vector for use in treating ischemic heart disease, peripheral vascular disease, cerebrovascular disease or preeclampsia in a cell or animal in need thereof.

In another aspect, the present disclosure provides an isolated nucleic acid molecule that silences the expression of the large intergenic RNA encoded by SEQ ID NO:1 (LIVE). In one embodiment, the isolated nucleic acid molecule targets the sequence AGGGAGCUGCUCCCUCUGCCAUGGUCA (SEQ ID NO:2). In another embodiment, the isolated nucleic acid molecule targets the sequence CAGCAGGAAAGGCUUGUGCGAAGGCUC (SEQ ID NO:3).

In an embodiment, the isolated nucleic acid molecule that silences the expression of LIVE is an antisense oligonucleotide or an siRNA molecule.

In one embodiment, the siRNA molecule is a double stranded RNA, Dicer substrate siRNA, shRNA or microRNA.

In an embodiment, the isolated siRNA molecule is optionally 19-49 nucleotides in length. In another embodiment, the siRNA molecule is optionally 21-27 nucleotides in length.

In a particular embodiment, the siRNA molecule is a double stranded siRNA molecule comprising the sense strand 5′UGACCAUGGCAGAGGGAGCAGCUCCCU3′ (SEQ ID NO:4) and the antisense strand 5′AGGGAGCUGCUCCCUCUGCCAUGGUCA3′ (SEQ ID NO:5) or comprising nucleic acids which are at least 75%, 80%, 85% 90% or 95% identical to SEQ ID NO: 4 or 5. In another embodiment U can be substituted with T.

In another particular embodiment, the siRNA molecule is a double stranded siRNA molecule comprising the sense strand 5′GAGCCUUCGCACAAGCCUUUCCUGCUG3′ (SEQ ID NO:6) and the antisense strand 5′CAGCAGGAAAGGCUUGUGCGAAGGCUC3′ (SEQ ID NO: 7) or comprising nucleic acids which are at least 75%, 80%, 85% 90% or 95% identical to SEQ ID NO: 6 or 7. In another embodiment U can be substituted with T.

The isolated nucleic acid molecule that silences the expression of LIVE is optionally chemically modified to increase stability.

Further provided herein is a pharmaceutical composition comprising an isolated nucleic acid molecule that silences the expression of LIVE disclosed herein and a pharmaceutically acceptable carrier or diluent. In an embodiment, the carrier is a nanoparticle for delivery of the molecule. In another embodiment, the isolated nucleic acid molecule that silences the expression of LIVE is a shRNA or antisense and is contained in an expression vector. In yet another embodiment, the pharmaceutical composition further comprises a poly(ADP-ribose) polymerase (PARP)1 inhibitor and/or an RNA helicase (RHA) inhibitor.

In yet another aspect, the present disclosure provides a method of inhibiting angiogenesis comprising administering to a cell or animal in need thereof an agent that inhibits the large intergenic RNA (LIVE) encoded by the nucleic acid sequence as shown in SEQ ID NO:1. Also provided is use of an agent that inhibits the large intergenic RNA (LIVE) encoded by the nucleic acid sequence as shown in SEQ ID NO:1 for inhibiting angiogenesis in a cell or animal in need thereof. Further provided is use of an agent that inhibits the large intergenic RNA (LIVE) encoded by the nucleic acid sequence as shown in SEQ ID NO:1 in the manufacture of a medicament for inhibiting angiogenesis in a cell or animal in need thereof. Even further provided is an agent that inhibits the large intergenic RNA (LIVE) encoded by the nucleic acid sequence as shown in SEQ ID NO:1 for use in inhibiting angiogenesis in a cell or animal in need thereof.

In one embodiment, the animal has a cancer associated with vascularization, such as melanoma, renal cell carcinoma, breast carcinoma, colon carcinoma, lung cancer or choriocarcinoma. In a particular embodiment, the animal has glioma, optionally glioblastoma.

In another embodiment, the animal has diabetic retinopathy, diabetic nephropathy, proliferative retinopathy, proliferative renal disease or wet age-related macular degeneration.

In an embodiment, the agent that inhibits LIVE in the methods and uses disclosed herein is an isolated nucleic acid molecule that silences the expression of LIVE as disclosed herein.

In a further embodiment, a PARP1 inhibitor and/or an RHA inhibitor is contemporaneously used or administered with the agent that inhibits LIVE.

In yet another aspect, the disclosure provides a method for classifying a clinical histopathological grade of glioma in a subject, comprising the steps:

(a) determining the expression of LIVE RNA in a sample from the subject; and

(b) comparing the expression of LIVE RNA from the sample with a control;

wherein the LIVE RNA is encoded by a nucleic acid comprising the nucleic acid sequence as shown in SEQ ID NO:1, and wherein a difference, or lack of difference, in the expression of LIVE RNA in the sample from the subject as compared to the control is indicative of the grade of glioma.

In another embodiment, the disclosure provides a method for classifying a change in the grade of glioma in a subject over time, comprising

(a) determining the expression of LIVE RNA in a sample from the subject at a first time point;

(b) determining the expression of LIVE RNA in a sample from the subject at a second time point; and

(c) comparing the expression of LIVE RNA in a sample from the first time point to the second time point;

wherein the LIVE RNA is encoded by a nucleic acid comprising the nucleic acid sequence as shown in SEQ ID NO:1, and wherein an increase in the expression level of LIVE RNA from the first time point to the second time point is indicative of an increase in the grade of glioma in the subject and a decrease in the expression level of LIVE RNA from the first time point to the second time point is indicative of a decrease in the grade of glioma in the subject.

Other features and advantages of the present disclosure will become apparent from the following detailed description. It should be understood, however, that the detailed description and the specific examples while indicating embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 shows relative transcript levels of control gene in 3D Matrigel and 2D HUVEC cultures with or without 100 ng/ml treatments of VEGF. RNA was collected from 2D and 3D culture and measured via qRT-PCR (N=4, mean±s.d.). Absolute copy number of cyclophilin A was obtained across samples to standardize for recovery efficiency and utilized as a negative control. A known amount of exogenous luciferase was added prior to RNA recovery to assess percent RNA recovery and first strand efficiency. Delta-Delta ct were obtained using cyclophilin A as normalization gene. Angiopoietin 2 and EGR3 were used as positive controls.

FIG. 2 shows VEGF-A₁₆₅ induces a distinct steady-state lncRNA profile within human umbilical vein endothelial cells in monolayer culture and in 3 dimensional vascular network formation. (A) The Venn diagram depicts the overlap between VEGF-responsive lncRNAs in 2D and 3D cultures. (B) The top 23 VEGF-responsive lncRNAs were chosen from both 2D and 3D culture and characterized by Ensembl/ENCODE. Pseudogenes and natural antisense transcripts were excluded. Fold change and P-value were used to select the top candidate genes. “Proximal” is defined by a 500 kb genomic window on either side of the protein-coding gene. Non-coding islands are defined as clusters of non-coding genes spanning 1 Mb or more, with no protein coding genes within the delineated region.

FIG. 3 shows the top 23 lncRNA candidates selected based on fold-change and P-value. (A) Top 23 VEGF responsive lncRNAs (which include lincRNAs) were chosen from both 2D and 3D culture and characterized by Ensembl/ENCODE. High fold change and low p-value were used to select the top candidate genes. LincRNA-VEGFR1 (“LIVE”, previously referred to as LIVE1) transcript is downregulated in the presence of VEGF-A₁₆₅ in 2D monolayer culture. (B) Microarray data from four biologically distinct samples demonstrates that lincRNA-VEGFR1 is significantly downregulated by VEGF-A₁₆₅ (100 ng/ml) in HUVECs cultured on gelatin. LincRNA-VEGFR1 decreases by 3.05 fold in raw hybridization intensity in 2D culture and increase with VEGF in 3D. Using student t-test, down regulation by VEGF in 2D has a p-value of 0.030. No change was observed for PDX1 across treatment conditions. Total VEGF receptor 1 exhibited down regulation in response to VEGF but change was not statistically significant. Pseudogenes and natural antisense transcripts were excluded. (C) No RT control was used for pre-mRNA measurements. LincRNA-VEGFR1 is enriched in VEGF responsive cell types such as endothelial cells. P-values were determined with student t-test and both p-value as well as mean±s.e.m. are shown above.

FIG. 4 shows LIVE is enriched in endothelial cells and RNAi knockdown of lincRNA-VEGFR1 inhibits VEGFR1 transcription and endothelial cell plexus stabilization. (A) RNA from biological triplicates from each cell type was obtained and measured via qRT-PCR with standard curves. Data were normalized to cyclophilin A and relative expression levels were obtained. (B) Dicer substrate siRNA (dsi-RNA) designed against lincRNA-VEGFR1 (dsi-RNA (I)) was transfected into HUVECs (N=3, mean±s.d.). RNA was collected at 48 h post-transfection and qRT-PCR was performed with standard curves for absolute quantification. 39% knockdown of lincRNA-VEGFR1 was observed in three separate experiments with three distinct biological samples. (C) Knockdown of lincRNA-VEGFR1 lead to significant reductions in VEGFR1 mRNA and the soluble variant sFLT levels (N=4). (D) Spheroid sprouting assay of siRNA transfected HUVECs, representative of three biologically distinct samples (N=3; scale bar=100 μm). (E) Time-lapse microscopy of vascular network formation on Matrigel with siRNA-transfected HUVECs, representative of three distinct biological samples. (P-value and mean±s.d. for all bar graphs are shown).

FIG. 5 shows human umbilical vein endothelial cells exposed to 0, 4 or 24 hours of hypoxia. All hypoxia experiments were carried out in Forma Anaerobic system (Thermo Fisher). A hypoxic environment (1% O₂) was achieved and maintained using a high purity anaerobic gas mixture (5% CO₂, 10% H₂, 85% N₂; Linde). Absolute RNA copy numbers were obtained by standard curves using qRT-PCR and normalized to a defined copy number of exogenous luciferase RNA in order to control for first strand synthesis efficiency as well as RNA recovery. 18S RNA level was used as control. LincRNA-VEGFR1 increased with 4 hours of hypoxia and was determined to have a p-value of 0.019 with paired student t-test (N=3, mean±s.d.). VEGFR1 pre-mRNA has a similar distribution with hypoxia treatment.

FIG. 6 shows a second Dicer substrate siRNA (dsi-RNA (II)) knockdown of lincRNA-VEGFR1 and the effects on VEGFR1 in HUVEC. dsiRNA (II) was assayed to ensure consistency using qRT-PCR and absolute quantification with standard curve (N=3).

FIG. 7 shows lincRNA-VEGFR1 knockdown inhibits angiogenic sprouting in spheroid model on collagen and Matrigel. HUVEC isolated from female and male births (N=3) was transfected with 40 uM of Dicer substrate siRNA against lincRNA-VEGFR1 (lincRNA-VEGFR1 siRNA) in 6 cm plates. The cells were harvested to generate 100 spheroids that were approximately 2250 cells per spheroid. (A) 26 hours after spheroid generation (34 h post-transfection), the 50 spheroids were collected and (B) seeded on collagen matrix (top panel) or Matrigel matrix (bottom panel). Sprouting was assayed after 24 hours post seeding in matrix (scale bar=100 μm). The number of sprouts were quantified by S.Core; P<0.05 versus scramble (student t-test). (C) The diameter of each spheroid was measured to assess whether cells within lincRNA-VEGFR1 knockdown spheroids were still viable after seeding. The knockdown spheroids continued to increase in diameter after plating, however, no sprouts formed (N=3, mean±s.d.).

FIG. 8 shows lincRNA-VEGFR1 is expressed at high levels in glioblastoma and enriched in GSCs and glioma-derived endothelial progenitor cells. (A) RNA was isolated from 16 gliomas (Grade I: 4 samples; Grade II: 2 samples; Grade III: 6 samples; GBM: 7 samples). Absolute copy number was determined with standard curve and all absolute quantification was normalized to % RNA recovery and first-strand efficiency. LincRNA-VEGFR1 is expressed at higher levels in glioblastoma compared to lower-grade gliomas; p-value was determined by unpaired student t-test (mean and iSEM shown). Each point represents an individual patient sample. Data for two glioblastoma samples were omitted as outliers. (B) Diagrammatic representation of lineage hierarchy of GSCs to endothelial cells as established by Wang et al. (2010). (C) Expression of lincRNA-VEGFR1 across the four intratumoural cell fractions, expressed in absolute copy number relative to 10,000 copies of GAPDH transcript. Each glioblastoma specimen is shown individually (representative figure; N=3).

FIG. 9 shows nuclear transfection of pEZM02-lincRNA-VEGFR1 in human umbilical endothelial cells and the effects of lincRNA-VEGFR1 over-expression on VEGFR-1 pre- and mature mRNA. (A) qRT-PCR was used to determine the absolute copy number of lincRNA-VEGFR1 as well as VEGFR1 pre- and mature mRNA in transfected HUVECs (N=3, mean±s.d.). P-values were determined by paired student t-test. Given that plasmid transfections lead to episomal expression of lincRNA-VEGFR1, the enhancer activity of lincRNA-VEGFR1 is not cis-restricted and distance dependent. (B) LincRNA-VEGFR1 over-expression increases the rate of elongation and plexus formation during vascular network formation (microvasculogenesis) on Matrigel, determined by time-lapse microscopy. Time-lapse images were taken from 2-16 hours. The number of tubes was calculated by S.Core; p=0.039 vs. Scramble (student t-test).

FIG. 10 shows expression of VEGFR1 transcriptional products across different grades of glioma tumours. RNA was isolated from 14 glioma tumours with exogenous luciferase RNA introduced in a known amount during extraction to control for total RNA recovery and first strand efficiency. Absolute copy number was determined with standard curve and all absolute quantifications were normalized to % RNA recovery and first strand efficiency.

FIG. 11 shows dsi-RNA (I) knockdown of lincRNA-VEGFR1 in GSCs and glioma-derived endothelial cell precursors inhibits VEGFR1 transcription and vascular network formation. (A) GliNS1 GSCs were treated with either standard stem cell medium or endothelial-conditioned medium and seeded on 3D Matrigel (N=4; scale bar=100 μm). (B) LincRNA-VEGFR1 expression was determined by absolute quantification using qRT-PCR. All values were normalized to exogenous luciferase RNA for RNA recovery and first strand efficiency. Fetal neural stem cells (FNS) were used as a control. (N=4, mean±s.d.) (C) Endothelial-differentiated GSCs were transfected and then seeded on to 3D Matrigel (N=4; scale bar=100 μm). Tube formation was quantified by S.Core; P=0.015 vs. scramble (student t-test). (D) GSCs were transfected with Dicer-substrate siRNA against lincRNA-VEGFR1. Absolute copy numbers of lincRNA-VEGFR1 transcripts as well as VEGFR1 and sFLT were obtained via qRT-PCR and normalized to exogenous luciferase (N=3, mean±s.d.). All p-values were determined by student t-test.

FIG. 12 shows GliNS1 CD133+GBM stem-like cells were treated with either standard stem cell medium or endothelial conditioned medium and seeded on 3D Matrigel (N=4, mean±s.d). (A) VEGFR1 and (B) eNOS expression was determined by absolute quantification with qRT-PCR, all values were normalized to exogenous luciferase RNA for RNA recovery and first strand efficiency. Transcription of endothelial nitric oxide synthase was used as a positive control for endothelial conditioned medium treatments. Fetal neural stem cells (FNS) were used as a negative control. P-values were determined by student t-test.

FIG. 13 shows nanoparticle-based, RNAi targeting of lincRNA-VEGFR1 results in decreased tumour volume and vascularity in glioblastoma xenograft tumours. (A) Nanoparticle-based delivery of dsiRNA (I) targeting lincRNA-VEGFR1 leads to 74% decrease in lincRNA-VEGFR1 abundance in the tumour core when compared to scrambled control (statistical significance was determined by unpaired student t-test, N=5; mean±SEM). (B,C) GliNS1 GSCs were used to generate xenograft tumours in NOD-SCID mice. Treatment with nanoparticle-based delivery of RNAi against lincRNA-VEGFR1 results in decreases in tumour weight and volume compared to treatment with nanoparticles carrying scrambled control. Treatment consisted of 5 intratumoural injections over 10 days (N=9 in each group, student t-test was used to determine statistical significance after correlation of weight and volume was established with linear regression). (D) Orthotopic xenografts created with injection of GliNS1 were treated with dsi-RNA (I) against lincRNA-VEGFR1 with a half-life of 24 hours delivered by ALZET osmotic pumps. Kaplan-Maier survival curves were plotted (N=5). (E) Co-immunofluorescence staining for PCNA and CD31 demonstrates a significant decrease in proliferation of perivascular glioma cells in RNAi-treated xenograft tumours (N=4; representative image, scale bar depicts 22 μm).

FIG. 14 shows nanoparticle-based delivery of RNAi against lincRNA-VEGFR1 results in a decrease in intra-tumoural microvasculature. (A) IHC for CD31 demonstrates a significant decrease in microvessel density (microvessels <100 μm in diameter). (B) Quantification of perfused vascular density as assessed by high molecular weight FITC-dextran (N=3; representative images, scale bar=200 μm). (For all box and whiskers plots lower quartile (Q1), median (Q2), upper quartile (Q3) shown). (C) LincRNA-VEGFR1 RNAi-treated glioblastoma xenograft tumours show a 53% reduction in the expression of VEGFR1 compared to control (representative image; N=6; student t-test used to determine statistical significance). (D) Double IHC staining for CD31 and cleaved caspase 3 demonstrates apoptotic cells near blood vessels in lincRNA-VEGFR1 RNAi-treated xenografts. Haematoxylin was used to visualize the nucleus (representative image; N=4, scale bar=200 μm). (E) Quantification of double IF for SOX2 and CD31 demonstrates a significant decrease in perivascular SOX2-positive cells in lincRNA-VEGFR1 RNAi-treated tumours (statistical significance was determined by student t-test; N=4; representative images, scale bar=22 μm). (F) Co-IF with CD31 and PDGFR-β or NG2 delineates blood vessels and pericytes in glioblastoma xenografts (statistical significance was determined by student t-test; N=3; representative images, scale bar=30μm). All statistical significance was determined by student t-test.

FIG. 15 shows effects of dsi-RNA (II) against lincRNA-VEGFR1 on GBM xenografts and the intratumoural distribution of Cy3-labeled dsi-RNA (II). (A) Subcutaneous xenograft tumours were injected with scrambled negative control delivered by nanoparticle vehicle. Tumour length and width were measured pre-injection and calculated by the equation [(width)²×length]/2 (N=4). (B) Cy3-labeled scrambled negative control was complexed with nanoparticle at the same concentration as treatment to visualize intra-tumoural localization (N=4; scale bar=120 μM).

FIG. 16 shows HIF-1α AND HIF-2α signatures within the xenograft tumours. No detectable difference was detected in the signatures amongst treatments (control versus dsi-RNA (I)). Absolute quantification of HIF target genes was obtained by qRT-PCR.

FIG. 17 shows microarray profiling of dsi-RNA (I)-mediated knockdown of lincRNA-VEGFR1 in HUVECs (N=4). (A) Western blot quantification of trans-membrane VEGFR1 in lincRNA-VEGFR1 knockdown lysates compared to scrambled control transfection lysates demonstrate a decrease in protein levels at 72 hours (N=4). (B) Four biological samples of HUVECs were subject to knockdown with either of the two dsi-RNAs designed against lincRNA-VEGFR1. With knockdown of lincRNA-VEGFR1 there were more protein encoding genes downregulated compared to those observed to be upregulated. (C) qRT-PCR was used to examine VE-Cadherin as well as VEGF-A expression. (D) Representative western blots for VEGFR2 and neuropilin 1 (N=4).

FIG. 18 shows LIVE enhances VEGFR1 transcription and knockdown of LIVE identifies additional gene targets. (A) Using absolute copy number quantification with qRT-PCR, top LIVE-regulated genes were assessed. Decreases in VEGFR2 mRNA expression were not significant due to baseline copy number variation across biological samples. Neuropilin-1 transcripts changed significantly by relative expression level, and VE-Cadherin transcript levels remained unchanged. mRNA levels of neighboring protein genes within the genomic region of LIVE and VEGFR1, such as GSX1, PDX1 and PAN3, were unchanged, consistent with microarray results from knockdown (N=4; mean±SEM, student t-test). (B) sFLT ELISA was used to determine the concentration of sFLT in HUVEC media in knockdown and control conditions (N=4; paired student t-test).

FIG. 19 shows results of overexpression of lincRNA-VEGFR1 in HUVECs. (A) Forty-eight hours post-transfection, protein lysates from HUVECs grown in 2D monolayer were collected. Lysates were probed for VEGFR1 and normalized to GAPDH for equal loading. No observable difference in VEGFR1 protein was observed (N=4; C=control vector only, E=experimental overexpression vector; representative blot shown). (B) Forty-eight hours post-transfection of overexpression vector was observed to result in a stable increase in VEGFR1 pre-mRNA transcriptional products, however, total mature mRNA products for VEGFR1 gene remained constant and did not increase (N=3, mean t s.d.).

FIG. 20 shows expression of VEGF-A, VEGFR2, and VEGFR1 transcriptional products in tumour xenograft cores. Mature mRNA of VEGF-A as well as VEGFR2 and VEGFR1 transcriptional products were measured in RNA isolated from the tumour core via qRT-PCR (N=5).

FIG. 21 shows effects of lincRNA-VEGFR1 knockdown on VEGF receptor expression levels in whole tumour extracts in GBM xenografts. A) Immunoblots of tumour core and periphery samples (N=5). B) Quantification of VEGFR1, VEGFR2 and NRP1 of immunoblots (+/−SEM, N=5).

FIG. 22 shows immunofluorescence images (top; LIVE/DEAD stain in GSCs) and graphs (bottom; graphical quantification of immunofluorescence images) which demonstrate that depletion of LIVE does not cause changes in glioma stem cell viability or vitality.

FIG. 23 shows (A) phase contrast and immunofluorescence images; (B) graphs showing expression of LIVE, GADPH, PDGFR-β, endoglin, desmin, PECAM-1 and NG2 mRNA; and (C) western blot analysis (left) and graphical representation of protein expression (right), all in low LIVE-expressing glioma stem cells engineered to overexpress LIVE (resulting in increased expression of pericyte and endothelial markers at both mRNA and protein levels).

FIG. 24 shows the results of RNA-binding protein immunoprecipitation using anti-RHA and anti-PARP antibodies in GLiNS1 lysates to confirm association with LIVE via absolute quantification qRT-PCR (N=3).

FIG. 25 shows LIVE associates with PARP1 to drive endothelial and pericyte gene expression in (A) GSCs and (B) HUVECs.

FIG. 26 shows that depletion of LIVE combined with PARP1-specific inhibitors leads to decreases in endothelial and pericyte gene expression in GSCs.

FIG. 27 shows immunofluorescence images which demonstrate GSCs give rise to vascular pericytes and not to endothelial cells in xenografts.

### DETAILED DESCRIPTION

To detect lincRNAs that are functionally important to human angiogenesis, the present inventors used a custom microarray to profile long noncoding transcripts in human vascular endothelium in two-dimensional versus three-dimensional pro-angiogenic cultures, with or without VEGF-A₁₆₅s. In doing so, the present inventors identified a VEGF-A-responsive lincRNA near the VEGFR1 gene, which was termed lincRNA-VEGFR1 or “LIVE”. This lincRNA-VEGFR1 was previously referred to as LIVE1 but due to knowledge of a different molecule with that name was changed to LIVE.

Through knockdown and over-expression studies, LIVE was found to exert transcriptional control over the VEGFR1 gene and direct angiogenesis in vitro. Furthermore, LIVE was found to be highly expressed in glioblastoma, and enriched in glioma stem cell (GSC) fractions and neoplastic endothelial progenitor populations. In vivo knockdown of LIVE in a glioblastoma xenograft model decreased microvascular density and tumour volume and slowed tumour progression. Inhibition of LIVE within tumor also depletes vascular pericytes and decreases the number of cancer stem cells. These results establish LIVE as a key mediator of angiogenesis and demonstrate the potential of lincRNA-based therapeutics.

### DEFINITIONS

As used herein “a”, “an” and/or “the” includes one and/or more than one.

The term “LIVE” or “lincRNA-VEGFR1” refers to a VEGF-A-responsive lincRNA near the VEGFR1 gene as shown in SEQ ID NO:8 and which is encoded by the DNA sequence shown in SEQ ID NO:1. This molecule was previously referred to as “LIVE1”.

The term an “agent that inhibits LIVE” as used herein refers to an agent that reduces or silences LIVE RNA expression.

The term “treatment or treating” as used herein means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable.

The term a “therapeutically effective amount”, “effective amount” or a “sufficient amount” of a compound of the present application is a quantity sufficient to, when administered to the subject, including a mammal, for example a human, effect beneficial or desired results, including clinical results, and, as such, an “effective amount” or synonym thereto depends upon the context in which it is being applied. For example, in the context of inhibiting LIVE expression, it is an amount of the agent sufficient to achieve such an inhibition as compared to the response obtained without administration of the agent. For example, in the context of overexpressing LIVE, it is an amount of the agent sufficient to achieve such overexpression as compared to the response obtained without administration of the agent. In the context of disease, therapeutically effective amounts of the agents are used to treat, modulate, attenuate, reverse, or affect angiogenesis in such diseases. An “effective amount” is intended to mean that amount of an agent that is sufficient to treat, prevent or inhibit such disorders, conditions or diseases. The amount of a given agent that will correspond to such an amount will vary depending upon various factors, such as the given agent, the pharmaceutical formulation, the route of administration, the type of condition, disease or disorder, the identity of the subject or host being treated, and the like, but can nevertheless be routinely determined by one skilled in the art. Also, as used herein, a “therapeutically effective amount” of an agent is an amount which prevents, inhibits, suppresses or reduces a disorder, disease or conditions that benefits from an inhibition of LIVE expression, for example, vascularized tumours, as determined by clinical symptoms, in a subject as compared to a control. Also, as used herein, a “therapeutically effective amount” of an agent is an amount which prevents, inhibits, suppresses or reduces a disorder, disease or conditions that benefits from overexpression of LIVE expression, for example, in diabetic nephropathy, as determined by clinical symptoms, in a subject as compared to a control. As defined herein, a therapeutically effective amount of an agent may be readily determined by one of ordinary skill by routine methods known in the art.

Moreover, a “treatment” or “prevention” regime of a subject with a therapeutically effective amount of an agent may consist of a single administration, or alternatively comprise a series of applications. For example, the agent may be administered at least once a week. However, in another embodiment, the agent may be administered to the subject from about one time per week to about once daily for a given treatment. The length of the treatment period depends on a variety of factors, such as the severity of the disease, the age of the patient, the concentration and the activity of the agent, or a combination thereof. It will also be appreciated that the effective dosage of the agent used for the treatment or prophylaxis may increase or decrease over the course of a particular treatment or prophylaxis regime. Changes in dosage may result and become apparent by standard diagnostic assays known in the art. In some instances, chronic administration may be required.

As used herein, the term “control” refers to a sample from a subject or a group of subjects who are either known as having a particular condition or trait or as not having a particular condition or trait. The control can vary depending on what is being monitored, assessed or diagnosed. For example, if one is monitoring the progression of a glioma, the control can be from a subject who is known to have a particular grade of glioma. In another embodiment, the control is from the cells of a subject or a group of subjects known to express a particular level or amount of LIVE RNA. The control can also be a predetermined standard or reference range of values.

The term “angiogenesis” as used herein refers to the creation of new blood vessels from either pre-existing vessels or nascent formation of vascular networks which then connect to pre-existing vessels.

The term “subject” or “animal” as used herein includes all members of the animal kingdom including mammals, suitably humans.

The term “administering” is defined as any conventional route for administering an agent to a subject for use, for example, in promoting or inhibiting angiogenesis, as is known to one skilled in the art. This may include, for example, administration via the parenteral (i.e. subcutaneous, intradermal, intramuscular, etc.) or mucosal surface route. In other embodiments this may include oral administration. The dose of the agent may vary according to factors such as the health, age, weight and sex of the animal. The dosage regime may be adjusted to provide the optimum dose. One skilled in the art will appreciate that the dosage regime can be determined and/or optimized without undue experimentation.

To “inhibit” or “suppress” or “lower” or “reduce” or “downregulate” a function or activity, such as LIVE RNA expression, is to reduce the function or activity when compared to otherwise same conditions except for a condition or parameter of interest, or alternatively, as compared to another condition or control.

The term “inhibiting angiogenesis” as used herein refers to decreasing the amount, extent or degree of angiogenesis.

To “promote” or “enhance” or “increase” or “upregulate” a function or activity, such as LIVE RNA expression, is to increase the function or activity when compared to otherwise same conditions except for a condition or parameter of interest, or alternatively, as compared to another condition or control.

The term “promoting angiogenesis” as used herein refers to increasing the amount, extent or degree of angiogenesis.

The term “nucleic acid” and/or “oligonucleotide” as used herein refers to a sequence of nucleotide or nucleoside monomers consisting of naturally occurring bases, sugars, and intersugar (backbone) linkages, and includes single stranded and double stranded molecules, RNA and DNA. The term also includes modified or substituted oligomers comprising non-naturally occurring monomers or portions thereof, which function similarly, which are referred to herein as “chemical analogues” and/or “oligonucleotide analogues” such as “peptide nucleic acids”. Such modified or substituted nucleic acids may be preferred over naturally occurring forms because of properties such as enhanced cellular uptake, or increased stability in the presence of nucleases. The term also includes chimeric nucleic acids that contain two or more chemically distinct regions. For example, chimeric nucleic acids may contain at least one region of modified nucleotides that confer beneficial properties (e.g. increased nuclease resistance, increased uptake into cells), or two or more nucleic acids of the disclosure may be joined to form a chimeric nucleic acid. The term “nucleic acid” includes for example, “antisense oligonucleotides”, “siRNA oligonucleotides”, and “miRNA” as well as oligonucleotide analogues such as “morpholino oligonucleotides”, “phosphorothioate oligonucleotides”, and “peptide nucleic acids”. The term “nucleic acid” also includes aptamers.

The term “isolated nucleic acid molecule” as used herein refers to a nucleic acid substantially free of cellular material or culture medium when produced by recombinant DNA techniques, or chemical precursors, or other chemicals when chemically synthesized. An isolated nucleic acid is also substantially free of sequences which naturally flank the nucleic acid (i.e. sequences located at the 5′ and 3′ ends of the nucleic acid) from which the nucleic acid is derived. The term “nucleic acid” is intended to include DNA and RNA and can be either double stranded or single stranded, and represents the sense or antisense strand.

The term “variant” as used herein includes modifications, substitutions, additions, derivatives, analogs, fragments or chemical equivalents of the nucleic acid sequences disclosed herein that perform substantially the same function in substantially the same way.

The terms “RNA interference,” “interfering RNA” or “RNAi” refer to single-stranded RNA or double-stranded RNA (dsRNA) that is capable of reducing or inhibiting expression of a target nucleic acid by mediating the degradation of mRNAs which are complementary to the sequence of the interfering RNA when the interfering RNA is in the same cell as the target gene. Interfering RNA may have substantial or complete identity to the target nucleic acid or may comprise a region of mismatch.

The term “siRNA” or “siRNA oligonucleotide” refers to a short inhibitory RNA that can be used to reduce or inhibit nucleic acid expression of a specific nucleic acid by RNA interference. For example siRNAs can be 19-49 nucleotide double stranded RNA molecules that correspond to a target region in a gene of interest (e.g. comprise a sense strand homologous to the target mRNA).

The siRNA can be a duplex, such as a Dicer substrate siRNA, a short RNA hairpin (shRNA) or a microRNA (miRNA).

A person skilled in the art will recognize that an RNA molecule can be altered by substituting uracil (U) with thymine (T) residues without abolishing the ability of the resulting molecule to inhibit RNA expression. Optionally, the nucleic acids disclosed herein are chemically synthesized. siRNA can also be generated by cleavage of longer dsRNA (e.g., dsRNA greater than about 25 nucleotides in length) with the E. coli RNase III or Dicer. These enzymes process the dsRNA into biologically active siRNA. The dsRNA can encode for an entire gene transcript or a partial gene transcript. In the case of a shRNA, the siRNA can be derived from DNA-based vectors or agents that synthesize shRNAs.

Methods of designing specific nucleic acid molecules that silence gene expression and administering them are known to a person skilled in the art. For example, it is known in the art that efficient silencing is obtained with siRNA duplex complexes paired to have a two nucleotide 3′ overhang. The siRNA can also be chemically modified to increase stability. For example adding two thymidine nucleotides and/or 2′O methylation is thought to add nuclease resistance. Other modifications include the addition of a 2′-O-methyoxyethyl, 2′-O-benzyl, 2′-O-methyl-4-pyridine, C-allyl, O-allyl, O-alkyl, O-alkylthioalkyl, O-alkoxylalkyl, alkyl, alkylhalo, O-alkylhalo, F, NH2, ONH2, O-silylalkyl, or N-phthaloyl group (see U.S. Pat. No. 7,205,399; Kenski et al. Mol. Ther. Nucl. Acids 1:1-8 (2012); Behlke, Oligonucleotides 18:305-320 (2008)). Other modifications include direct modification of the internucleotide phosphate linkage, for example replacement of a non-bridging oxygen with sulfur, boron (boranophosphate), nitrogen (phosphoramidate) or methyl (methylphosphonate). A person skilled in the art will recognize that other nucleotides can also be added and other modifications can be made. As another example deoxynucleotide residues (e.g. dT) can be employed at the 3′ overhang position to increase stability.

The term “Dicer substrate siRNA” as used herein refers to a 25- to 35-mer that acts as a direct substrate for Dicer and thereby mediates gene silencing from endogenous RNAi pathways. As used herein in the examples, the terms “dsi-RNA”, “RNAi”, “siRNA” all refer to the Dicer substrate siRNA disclosed in Table 1.

The term “antisense oligonucleotide” as used herein refers to a nucleic acid molecule which is complementary to a “sense” nucleic acid molecule encoding an RNA, e.g., complementary to the coding strand of a double-stranded cDNA molecule or complementary to an mRNA sequence. Accordingly, an antisense nucleic acid can hydrogen bond to a sense nucleic acid. For example the nucleic acid can comprise DNA, RNA or a chemical analog, that binds to the messenger RNA produced by the target nucleic acid. Binding of the antisense nucleic acid prevents translation and thereby inhibits or reduces target protein expression. Antisense nucleic acid molecules may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine-substituted nucleotides. The antisense nucleic acid can be complementary to an entire target gene coding strand, or only to a portion thereof. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

The term “miRNA” refers to microRNAs which are small non-coding RNA molecules, for example 22 nucleotides, that are processed from hairpin RNA precursors, for example about 70 nucleotides long. miRNAs can inhibit gene expression through targeting homologous mRNAs.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log 10 [Na+])+0.41(% (G+C)−600/1), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In some embodiments, stringent hybridization conditions are selected. By way of example the following conditions may be employed to achieve stringent hybridization: hybridization at 5× sodium chloridelsodium citrate (SSC)/5×Denhardt's solution/1.0% SDS at Tm−5° C. based on the above equation, followed by a wash of 0.2×SSC/0.1% SDS at 60° C. Moderately stringent hybridization conditions include a washing step in 3×SSC at 42° C. It is understood, however, that equivalent stringencies may be achieved using alternative buffers, salts and temperatures. Additional guidance regarding hybridization conditions may be found in: Current Protocols in Molecular Biology, John Wiley & Sons, N.Y., (1989, 2002), and in: Sambrook et al., Molecular Cloning: a Laboratory Manual, Cold Spring Harbor Laboratory Press, (2001).

The term “substantially identical” or “essentially identical” as used herein means a nucleic acid sequence that, when optimally aligned, for example using the methods described herein, share at least 75%, 80%, 85%, 90%, 95%, 96%, 97%, 98%, 99%, or 100% sequence identity with a second nucleic acid sequence.

The term “sequence identity” as used herein refers to the percentage of sequence identity between two nucleotide sequences.

The term “pharmaceutically acceptable” means compatible with the treatment of animals, suitably humans.

The term “a cell” as used herein includes a plurality of cells and refers to all vascular or endothelial cells including pericytes. Administering a compound to a cell includes in vivo, ex vivo and in vitro treatment.

### Nucleic Acids

The present disclosure provides an isolated nucleic acid encoding a large intergenic RNA (LIVE), wherein the nucleic acid comprises the nucleic acid sequence as shown in SEQ ID NO:1 or a variant thereof.

Also provided is a vector comprising the nucleic acid encoding LIVE RNA and the necessary regulatory sequences for the transcription of the inserted sequence.

Suitable regulatory sequences may be derived from a variety of sources, including bacterial, fungal, viral, mammalian, or insect genes (for example, see the regulatory sequences described in Goeddel, Gene Expression Technology: Methods in Enzymology 185, Academic Press, San Diego, Calif. (1990)). Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, elements within the genomic context and sequences conferring inducibility of transcription may be incorporated into the expression vector. It will also be appreciated that the necessary regulatory sequences may be supplied by LIVE sequences and/or its flanking regions.

Recombinant expression vectors can be introduced into host cells to produce a transformed host cell. The term “transformed host cell” is intended to include cells that are capable of being transformed or transfected with a recombinant expression vector of the disclosure. The terms “transduced”, “transformed with”, “transfected with”, “transformation” and “transfection” are intended to encompass introduction of nucleic acid (e.g. a vector or naked RNA or DNA) into a cell by one of many possible techniques known in the art. Prokaryotic cells can be transformed with nucleic acid by, for example, electroporation or calcium chloride-mediated transformation. For example, nucleic acid can be introduced into mammalian cells via conventional techniques such as calcium phosphate or calcium chloride co-precipitation, DEAE-dextran mediated transfection, lipofectin, electroporation, microinjection, RNA transfer, DNA transfer, artificial chromosomes, viral vectors and any emerging gene transfer technologies. Suitable methods for transforming and transfecting host cells can be found in Sambrook et al. (Molecular Cloning: A Laboratory Manual, 2nd Edition, Cold Spring Harbor Laboratory press (1989)), and other laboratory textbooks.

Accordingly, further provided is a host cell transformed with the vector.

Suitable expression vectors for directing expression in mammalian cells generally include a promoter (e.g., derived from viral material such as polyoma, Adenovirus 2, cytomegalovirus and Simian Virus 40), as well as other transcriptional and translational control sequences. Examples of mammalian expression vectors include pCDM8 (Seed, B., Nature 329:840 (1987)), pMT2PC (Kaufman et al., EMBO J. 6:187-195 (1987)) and pCMV (Clontech, California, U.S.A.). Expression vectors specific for siRNA include, for example, pSilencer 2.1-U6 vectors (Life Technologies). pRNA-U6.1 as well as other vectors available from GenScript may be used for expression of siRNA (http://www.genscript.com/mai_vector.html).

In another aspect, the present disclosure provides an isolated nucleic acid molecule, such as an siRNA or antisense oligonucleotide, that silences the expression of the large intergenic RNA encoded by SEQ ID NO:1 (LIVE).

In one embodiment, the isolated nucleic acid molecule that silences the expression of LIVE targets the sequence AGGGAGCUGCUCCCUCUGCCAUGGUCA (SEQ ID NO:2).

In another embodiment, the isolated nucleic acid molecule that silences the expression of LIVE targets the sequence CAGCAGGAAAGGCUUGUGCGAAGGCUC (SEQ ID NO:3).

In an embodiment, the isolated nucleic acid molecule that silences the expression of LIVE is an isolated siRNA molecule. The isolated siRNA molecule is optionally 19-49 nucleotides in length. In one embodiment, the siRNA molecule is 25-29 nucleotides in length.

In a particular embodiment, the siRNA molecule is a double stranded siRNA molecule comprising the sense strand 5′UGACCAUGGCAGAGGGAGCAGCUCCCU3′ (SEQ ID NO:4) and the antisense strand 5′AGGGAGCUGCUCCCUCUGCCAUGGUCA3′ (SEQ ID NO:5) or comprising nucleic acids which are at least 75%, 80%, 85% 90% or 95% identical to SEQ ID NO: 4 or 5; wherein U can also be T.

In another particular embodiment, the siRNA molecule is a double stranded siRNA molecule comprising the sense strand 5′ GAGCCUUCGCACAAGCCUUUCCUGCUG3′ (SEQ ID NO:6) and the antisense strand 5′CAGCAGGAAAGGCUUGUGCGAAGGCUC3′ (SEQ ID NO: 7) or comprising nucleic acids which are at least 75%, 80%, 85% 90% or 95% identical to SEQ ID NO: 6 or 7, wherein U can also be T.

The isolated nucleic acid molecule that silences the expression of LIVE, such as siRNA molecule, is optionally chemically modified to increase stability as disclosed herein.

Nucleic acids of the disclosure also include variant nucleic acids that comprise at least 70%, 80%, 90%, 95%, 98%, 99% or 100% nucleic acid sequence identity with the nucleic acid molecules of the disclosure that retain inhibition, e.g. nucleic acid molecules that silence the expression of LIVE, when transcribed or that retain promotion, e.g. LIVE RNA overexpression. For example, the variant nucleic acid in one embodiment comprises a nucleic acid sequence that is at least 70%, 80%, 90%, 95%, 98%, 99% or 100% identical to a nucleic acid sequence selected from the group consisting of SEQ ID NOs: 1 or 4-7. Such variant nucleic acid sequences include nucleotide sequences that hybridize to the nucleic acids corresponding to SEQ ID NOs: 1 or 4-7 under at least moderately stringent hybridization conditions.

To determine the percent identity of two sequences, the sequences are aligned for optimal comparison purposes (e.g., gaps can be introduced in the sequence of a first amino acid or nucleic acid sequence for optimal alignment with a second amino acid or nucleic acid sequence). The amino acid residues at corresponding amino acid positions are then compared in the case of a protein sequence. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecule of the present disclosure. BLAST protein searches can be performed with the XBLAST program parameters set, e.g., to score-50, wordlength=3 to obtain amino acid sequences homologous to a protein molecule of the present disclosure. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

Nucleic acid molecules of the disclosure can be constructed using chemical synthesis and enzymatic ligation reactions using procedures known in the art. For example, LIVE RNA or nucleic acid molecules that silence the expression of LIVE can be chemically synthesized using naturally occurring nucleotides or variously modified nucleotides designed to increase the biological stability of the molecules or to increase the physical stability of the duplex formed between the antisense and sense nucleic acids in the case of the siRNA, e.g., phosphorothioate derivatives and acridine-substituted nucleotides can be used.

Examples of modified nucleotides which can be used to generate the nucleic acids disclosed herein include xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8 amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine. Alternatively, the nucleic acid molecules can be produced biologically using an expression vector.

Another example of a modification is to include modified phosphorous or oxygen heteroatoms in the phosphate backbone, short chain alkyl or cycloalkyl intersugar linkages or short chain heteroatomic or heterocyclic intersugar linkages in the nucleic acid molecules. For example, the nucleic acid sequences may contain phosphorothioates, phosphotriesters, methyl phosphonates, and phosphorodithioates.

A further example of an analog of a nucleic acid molecule of the disclosure is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497). PNA analogs have been shown to be resistant to degradation by enzymes and to have extended lives in vivo and in vitro. PNAs also bind stronger to a complementary DNA sequence due to the lack of charge repulsion between the PNA strand and the DNA strand. Other nucleic acid analogs may contain nucleotides containing polymer backbones, cyclic backbones, or acyclic backbones. For example, the nucleotides may have morpholino backbone structures (U.S. Pat. No. 5,034,506). The analogs may also contain groups such as reporter groups, a group for improving the pharmacokinetic or pharmacodynamic properties of nucleic acid sequence.

The nucleic acids such as siRNA oligonucleotides can be administered by an implantable minipump such as by using Alzet osmotic minipumps implanted subcutaneously in animals. Other implantable osmotic pumps, such as Rose-Nelson Pump, Higuchi Leeper Pump, Higuchi Theuwes pump, Implantable Miniosmotic pump and the DUROS system for human parenteral delivery, or oral osmotic pumps may be used (Gupta et al., IJCP, 2011, 6(1):1-8). Oligonucleotides can be delivered using various in vivo transfection agents such as nanoparticles and lipid-based reagents (e.g. liposomes) in humans using intratumoral, intracerebroventricular, intravenous or intra-arterial injections. Oligonucleotides are safe and nontoxic and could be delivered orally, by skin patches or using an implantable pump.

### Pharmaceutical Compositions

The agents for use in the methods and uses of the disclosure are suitably formulated into pharmaceutical compositions for administration to subjects, for example human subjects, in a biologically compatible form suitable for administration in vivo. Accordingly, also provided herein is a pharmaceutical composition comprising an isolated nucleic acid molecule that silences the expression of LIVE molecule disclosed herein and a pharmaceutically acceptable carrier or diluent. Further provided is a pharmaceutical composition comprising an isolated nucleic acid molecule that encodes for LIVE and comprises the nucleic acid sequence as shown in SEQ ID NO:1.

In another embodiment, the pharmaceutical composition further comprises a PARP1 inhibitor and/or an RHA inhibitor. Such inhibitors may be any agent capable of reducing the expression, activity or functionality of PARP1 and/or RHA and include antisense oligonudeotides, siRNA, shRNA and antibodies as well as small molecules. PARP1 inhibitors are known in the art and include BYK204165, BSI-201, AG014699, rucaparib (CO-338), olaparib (AZD-2281), veliparib (ABT-888), niraparib (MK4827), BMN 673, CEP-9722, CEP8983 and E7016 (Kummar et al., BMC Medicine 10:25, 2012). Poly (ADP-Ribose) Polymerase 1 may be from any source or organism. In one embodiment, PARP1 has the sequence set forth in gene bank accession no. NM_001618 (nucleotide) and NP_001609.2 (protein). RNA helicase A may be from any source or organism. In one embodiment, RHA (also known as DHX9) has the sequence set forth in gene bank accession number: NM_001357.4 (nucleotide) and NP_001348.2 (protein).

The compositions containing the agents can be prepared by known methods for the preparation of pharmaceutically acceptable compositions which can be administered to subjects, such that an effective quantity of the active agent is combined in a mixture with a pharmaceutically acceptable vehicle. Suitable vehicles are described, for example, in Remington's Pharmaceutical Sciences (2003-20th edition) and in The United States Pharmacopeia: The National Formulary (USP 24 NF19) published in 1999. On this basis, the compositions include, albeit not exclusively, solutions of the agents in association with one or more pharmaceutically acceptable vehicles or diluents, and contained in buffered solutions with a suitable pH and iso-osmotic with the physiological fluids.

In an embodiment, the carrier is a nanoparticle for delivery. The described nanoparticle is a polymer-based vehicle that can be administered intravenously, intraarterially, intracerebroventricularly, or intratumorally. Dicerna's EnCore lipid nanoparticles (http://www.dicema.com/approach-about-Inp.php) can be used to deliver the nucleic acids disclosed herein, such as the siRNAs, of the present disclosure. Multifunctional nanoparticles for delivery of nucleic acids disclosed herein, such as siRNA, can be polymeric. Non-limiting examples of cationic polymers for delivery of nucleic acids disclosed herein, such as siRNA, include poly(ethylenimine) (PEI), poly-L-lysine (PLL), mPEG45 b-PCL100-b-PPEEA12, and poly (beta-amino ester). Non-limiting examples of non-cationic polymers include poly(isobutyl cyanoacrylate) and poly(lactic-co-glycolic acid) (PLGA) (Gao et al., Methods Mol Biol, 2010, 629:53-67).

In the case of shRNA, the carrier can be a DNA-based expression vector for delivery, said vector comprising the necessary elements for expressing the shRNA molecule in a target cell (see Marsden, N Engl J Med, 2006, 355(9):953-954).

In accordance with the methods and uses of the disclosure, the described agents, salts or solvates thereof may be administered to a subject in a variety of forms depending on the selected route of administration, as will be understood by those skilled in the art. The compositions may be administered, for example, by oral, parenteral, buccal, sublingual, nasal, rectal, patch, pump or transdermal (topical) administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration may be by continuous infusion over a selected period of time.

The agents may be administered to a subject alone or in combination with pharmaceutically acceptable carriers, as noted above, and/or with other pharmaceutically active agents for the treatment of a disorder, disease or condition associated with aberrant angiogenesis, the proportion of which is determined by the solubility and chemical nature of the agents, chosen route of administration and standard pharmaceutical practice.

When used in combination with other agents useful in treating aberrant angiogenesis, for example, PARP1 inhibitors and/or RHA inhibitors, the agents disclosed herein are suitably administered contemporaneously with those other agents. As used herein, “contemporaneous administration” of two substances to an individual means providing each of the two substances so that they are both biologically active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances.

The dosage of the agents and/or compositions can vary depending on many factors such as the pharmacodynamic properties of the agent, the mode of administration, the age, health and weight of the recipient, the nature and extent of the symptoms, the frequency of the treatment and the type of concurrent treatment, if any, and the clearance rate of the compound in the animal to be treated. One of skill in the art can determine the appropriate dosage based on the above factors. The agents may be administered initially in a suitable dosage that may be adjusted as required, depending on the clinical response. For ex vivo treatment of cells over a short period, for example for 30 minutes to 1 hour or longer, higher doses of agent may be used than for long term in vivo therapy.

### Methods and Uses

In another aspect, there is provided a method of promoting angiogenesis in a cell or animal in need thereof comprising administering to the cell or animal in need thereof the nucleic acid encoding the LIVE RNA or variant thereof, the vector comprising the nucleic acid or the host cell transformed with the vector. Also provided is a use of the nucleic acid encoding the LIVE RNA or variant thereof, the vector comprising the nucleic acid or the host cell transformed with the vector for promoting angiogenesis in a cell or animal in need thereof. Further provided is use of the nucleic acid encoding the LIVE RNA or variant thereof, the vector comprising the nucleic acid or the host cell transformed with the vector in the manufacture of a medicament for promoting angiogenesis in a cell or animal in need thereof. Even further provided is the nucleic acid encoding the LIVE RNA or variant thereof, the vector comprising the nucleic acid or the host cell transformed with the vector for use in promoting angiogenesis in a cell or animal in need thereof.

In an embodiment, “promoting angiogenesis” means increasing the extent of angiogenesis by 10%, 20%, 30% or more compared to a control that has not been treated with LIVE RNA.

In an embodiment, the methods and uses for promoting angiogenesis are useful in treating ischemic heart disease, peripheral vascular disease, cerebrovascular disease or preeclampsia. Accordingly, also provided herein is a method of treating ischemic heart disease, peripheral vascular disease, cerebrovascular disease or preeclampsia comprising administering to the cell or animal in need thereof the nucleic acid encoding the LIVE RNA or variant thereof, the vector comprising the nucleic acid or the host cell transformed with the vector. Also provided is a use of the nucleic acid encoding the LIVE RNA or variant thereof, the vector comprising the nucleic acid or the host cell transformed with the vector for treating ischemic heart disease, peripheral vascular disease, cerebrovascular disease or preeclampsia in a cell or animal in need thereof. Further provided is use of the nucleic acid encoding the LIVE RNA or variant thereof, the vector comprising the nudeic acid or the host cell transformed with the vector in the manufacture of a medicament for treating ischemic heart disease, peripheral vascular disease, cerebrovascular disease or preeclampsia in a cell or animal in need thereof. Even further provided is the nucleic acid encoding the LIVE RNA or variant thereof, the vector comprising the nucleic acid or the host cell transformed with the vector for use in treating ischemic heart disease, peripheral vascular disease, cerebrovascular disease or preeclampsia in a cell or animal in need thereof.

In yet another aspect, the present disclosure provides a method of inhibiting angiogenesis comprising administering to a cell or animal in need thereof an agent that inhibits the large intergenic RNA (LIVE) encoded by the nucleic acid sequence as shown in SEQ ID NO:1. Also provided is use of an agent that inhibits the large intergenic RNA (LIVE) encoded by the nucleic acid sequence as shown in SEQ ID NO:1 for inhibiting angiogenesis in a cell or animal in need thereof. Further provided is use of an agent that inhibits the large intergenic RNA (LIVE) encoded by the nucleic acid sequence as shown in SEQ ID NO:1 in the manufacture of a medicament for inhibiting angiogenesis in a cell or animal in need thereof. Even further provided is an agent that inhibits the large intergenic RNA (LIVE) encoded by the nucleic acid sequence as shown in SEQ ID NO:1 for use in inhibiting angiogenesis in a cell or animal in need thereof.

In an embodiment, “inhibiting angiogenesis” means reducing the extent of angiogenesis by 10%, 20%, 30% or more compared to a control that has not been treated with an agent that inhibits LIVE RNA expression.

In one embodiment, the animal has a cancer associated with vascularization, such as melanoma, renal cell carcinoma, breast carcinoma, colon carcinoma, lung cancer or choriocarcinoma. In a particular embodiment, the animal has glioblastoma.

In an embodiment, the animal has diabetic retinopathy, diabetic nephropathy, proliferative retinopathy, proliferative renal disease or wet age-related macular degeneration (AMD).

In an embodiment, the agent that inhibits LIVE in the methods and uses disclosed herein is an isolated nucleic acid molecule that silences the expression of LIVE disclosed herein.

In one embodiment, the isolated nucleic acid molecule that silences the expression of LIVE is an antisense oligonucleotide or an siRNA molecule.

In one embodiment, the isolated nucleic acid molecule that silences the expression of LIVE molecule is an siRNA molecule. The isolated siRNA molecule is optionally 19-49 nucleotides in length and optionally chemically modified to increase siRNA stability.

In another embodiment, the methods and uses for inhibiting angiogenesis further comprise contemporaneously administering a PARP1 inhibitor and/or an RHA inhibitor as disclosed herein.

In yet another aspect, the disclosure provides a method for classifying a clinical histopathological grade of glioma in a subject, comprising the steps:

(a) determining the expression of LIVE RNA in a sample from the subject; and

(b) comparing the expression of LIVE RNA from the sample with a control;

wherein the LIVE RNA is encoded by a nucleic acid comprising the nucleic acid sequence as shown in SEQ ID NO:1, and wherein a difference, or lack of difference, in the expression of LIVE RNA in the sample from the subject as compared to the control is indicative of the grade of glioma.

In another embodiment, the disclosure provides a method for determining a change in the grade of glioma in a subject over time, comprising

(a) determining the expression of LIVE RNA in a sample from the subject at a first time point;

(b) determining the expression of LIVE RNA in a sample from the subject at a second time point; and

(c) comparing the expression of LIVE RNA in a sample from the first time point to the second time point;

wherein the LIVE RNA is encoded by a nucleic acid comprising the nucleic acid sequence as shown in SEQ ID NO:1, and wherein an increase in the expression level of LIVE RNA from the first time point to the second time point is indicative of an increase in the grade of glioma in the subject and a decrease in the expression level of LIVE RNA from the first time point to the second time point is indicative of a decrease in the grade of glioma in the subject.

The inventors identified that various grades of glioma tumour cells express high levels of LIVE RNA. Thus, wherein the control is from a normal subject, known to be healthy and not have glioma, increased LIVE RNA expression level in cells from the subject as compared to the control indicates that the subject has glioma. In another example, wherein the control is a reference standard known to be indicative of a healthy individual not having glioma, increased LIVE RNA expression level from the subject as compared to the control indicates that the subject indicates that the subject has glioma.

In an embodiment, wherein the control is a reference standard known to be indicative of a subject with a particular grade of glioma, LIVE RNA expression level in cells from the subject similar to the control is indicative that the subject has the same grade of glioma. In an embodiment, wherein the control is a reference standard known to be indicative of a subject with a particular grade of glioma, LIVE RNA expression level in cells from the subject different from the control is indicative that the subject has a different grade of glioma.

The above disclosure generally describes the present application. A more complete understanding can be obtained by reference to the following specific examples. These examples are described solely for the purpose of illustration and are not intended to limit the scope of the disclosure. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Materials and Methods

RNA Extraction and qRT-PCR.

RNA was extracted from HUVEC isolated from female and male births cultured in 2D conventional culture and 3D Matrigel (BD). Cells were recovered from 3D Matrigel using a 3D cell collection kit (Trevigen). RNA was then recovered from samples using RNeasy mini plus kit (Qiagen). Exogenous luciferase RNA was introduced in a known amount during extraction to assess RNA recovery and first strand efficiency. 500 ng-1,000 ng of total RNA was used to synthesize random primed cDNA using SuperScriptIII First-strand synthesis SuperMix or Superscript VILO (Invitrogen). Absolute quantification of transcripts was performed with standard curves using Power SYBR Green (Applied Biosystems). No reverse transcriptase controls were used for all pre-mRNA measurements along with normal controls. ABI Prism and Viia7 were used and ct1000 (ct measurements for 1000 copies) were compared along with standard curves across machines to insure consistency. For lincRNA measurements, since RNA is more stable than single stranded DNA, cDNA was synthesized directly prior to use.

Microarray Analysis.

RNA was collected from 2D and 3D culture from four distinct biological samples of HUVECs (male and female births). Each biological sample was subjected to four treatment conditions, mock treatment or 100 ng/ml of VEGF-A₁₆₅ every 12 hours in 2 dimensional monolayer gelatin culture as well as 3 dimensional Matrigel culture (tumor-derived extracellular matrix that induces vascular network formation) and supplemented 30 minutes pre-collection. Same procedure was used for knockdown versus scrambled control experiments (n=4). Prior to submission to microarray, absolute copy number of cyclophilin A was measured via qRT-PCR across samples to standardize for recovery efficiency and utilized as a negative control. A known amount of exogenous synthesized luciferase RNA was added prior to RNA recovery to assess percent RNA recovery and first strand efficiency. Delta-delta CTs were obtained using cyclophilin A as a normalization gene. Angiopoietin 2 and eNOS were used as positive controls, since they have been shown to be VEGF responsive in previous literature (Abe and Sato 2001; Wary, Thakker et al. 2003). Agilent array platform was employed for microarray analysis. The sample preparation and microarray hybridization were performed based on the manufacturer's standard protocols with minor modifications. Briefly, mRNA was purified from 1 μg total RNA after removal of rRNA (mRNA-ONLY™ Eukaryotic mRNA Isolation Kit, Epicentre). Then, each sample was amplified and transcribed into fluorescently labeled cRNA. The labeled cRNAs were then hybridized to 8×60K Human LncRNA Array v2 in Agilent hybridization system to detect the expression of 33,045 LncRNA transcripts and 30,215 protein coding transcripts (Arraystar, Rockville, Md.). For each individual transcript, a specific exon or splice junction probe was designed. After hybridization and washing, the processed slides were scanned with using the Agilent DNA Microarray Scanner G2505C. The Agilent Feature Extraction software (version 10.7.3.1) was used to analyze acquired array images.

Statistical Analysis.

Arraystar Human LncRNA Microarray v2.0 was analyzed in GeneSpring GX v11.5.1. The microarray probe intensities were normalized using quantile method and then log transformed. The differentially expressed genes with statistical significance (including LncRNAs and mRNAs) were identified by Volcano Plot screening (two-tailed Student's test, equal variance, and no correction). Unsupervised hierarchical clustering and supervised hierarchical clustering of LncRNAs and mRNAs expression profiles was performed using Genespring GX v11.5.1.Venn Plot analysis in Genespring GX v11.5.1 was used to obtain the differentially expressed LncRNAs or mRNAs in both groups. Enrichment Analysis was performed using differentially expressed mRNAs towards GO terms and KEGG (Kyoto Encyclopedia of Genes and Genomes) pathway database (Ashbumer, Ball et al. 2000). Statistical significance was measured in Fisher's exact test (P-value <=0.05). Statistical analysis for all subsequent data was performed by Prism 6 (GraphPad). For comparisons of two groups, unpaired and two-tailed student's t-test was used. A value of P<0.05 was considered significant.

5′ Rapid Amplification of cDNA Ends (5′RACE). 5′ RACE was performed on 2 g of total extracted HUVEC RNA using 2^{nd} Generation 5′RACE kit (Roche). The 3 specific primers used were designed upstream of the siRNA site towards the 5′ end of the gene. High Pure PCR Product Purification kit (Roche) was used for the isolation of gene specific first strand (Table 1).

RNA Interference.

Cy3-labeled Dicer substrate siRNA was used to visualize transfection efficiency, transfection efficiency was determined. Dicer substrate siRNA designed against LIVE and Dicer substrate universal negative control (scrambled) were used at 401M per 6 cm plate. RNA was then collected at 48 hour post-transfection and qRT-PCR was performed with standard curves for quantification. Detailed protocol is as follows: 24 hrs before transfection, split HUVECs (grown on 100 mm dishes) into 60 mm dishes (˜6×10⁵ cells per plate) such that cells will be 85-95% confluent the next day. Use antibiotics-free HUVEC media.

Hypoxia Experiment.

All hypoxia experiments were carried out in Forma Anaerobic system (Thermo Fisher). A hypoxic environment (1% O₂) was achieved and maintained using a high purity anaerobic gas mixture (5% CO₂, 10% H₂, 85% N₂; Linde). HUVECs were subject to normoxia verses 0, 4 and 24 hours in hypoxic conditions, as previously described (Ho, Metcalf et al. 2012).

Vascular network formation on Matrigel. For knockdown studies, HUVECs were transfected with 40 μM of Dicer substrate siRNA against lincRNA-VEGFR1 in 6 cm plates (n=3). 24 hours after transfection, cells were plated on Matrigel on 6-well plates as previously described (Amaoutova and Kleinman 2010). Time-lapse images were taken from 4-17 hours post-seeding every 5 minutes on Zeiss widefield live-cell system. Images for scrambled control and lincRNA-VEGFR1 siRNA were taken simultaneously in 6-well format. For over expression studies, HUVECs (N=3) were transfected with either pEZM02-lncRNA-VEGFR1 or vector only control in 6 cm plates. Ten hours post-transfection, cells (counted by Milipore cell sorter) were plated on Matrigel coated 6 cm plates. Time-lapse images were taken every 5 minutes, from 2 hours post plating on Matrigel to 24 hours on Zeiss widefield live-cell system. Images for vector only control and lincRNA-VEGFR1 over-expression were taken simultaneously in 6-well format. S.Core was used to assess plexus formation and p-value was analyzed by student t-test.

Western Blot.

HUVECs were grown in M199 (Gibco 12340-030) containing antibiotics and 20% FBS at 37° C., 5% CO₂. Cells were grown to 80% confluency, washed with PBS, trypsinized, washed 2× with complete media (no antibiotics) and resuspended at 1×10⁶ cells/mi. 20 μg total of pEZ-M02 lincRNA-VEGFR1 or vector alone was added to 400 μl of the cell suspension, placed in a BTX disposable cuvette, 4 mm gap size and incubated for 5 min at room temperature. Cells were then electroporated by subjecting them to 200V, LV mode, for 70 msec using a BTX ECM830 Electrosquare Porator. Cells were incubated 5 min at room temperature before plating and growth in DME/F12 with 10% FBS. Cells were harvested at 24 and 48 hr post-electroporation by scraping in cell lysis buffer with protease inhibitors (Cell Signalling) vortexing for 10 sec and incubation on ice for 10 min. Cell debris was sedimented by centrifugation at 12,000×g for 10 min at 4° C. The supernatant was collected and quantified using the Pierce (Rockford, Ill.) BCA Protein Assay Kit and normalized for equal loading for SDS-PAGE and western blot analysis. 25 or 50 μg of cell lysates were separated by SDS-PAGE, transferred to nitrocellulose, PonceauS-stained and immunoblotted. The commercial primary antibodies used included anti-Flk1 (Santa Cruz; 1:1000), anti-NRP1 (cell signaling; 1:500), anti-VEGFR1 (AbCam #ab-32152) rabbit anti-human polyclonal and anti-lamin A/C (Santa Cruz #SC-7293) mouse anti-human monoclonal antibodies. Secondary antibodies were HRP-linked anti-rabbit or anti-mouse (used at 1:5,000 dilution) and protein bands detected with Biorad ECL (Biorad Laboratories, Hercules, Calif.). Quantification of the chemiluminescent signal from western blots was performed with Bio-Rad Fluor S Max Acquisition System (Biorad Laboratories) and Image Lab software (Biorad Laboratories).

sFLT ELISA.

Quantikine ELISA (R&D Systems; DVR100B) was used and the assay was performed according to manufacturer's instructions. SpectraMas M5e was used to assess optical density as well as establish standard curve. Media was collected at 48 hr and 72 hr post-knockdown and centrifuged.

Transfection for Episomal Expression.

BTX electroporation as well as lipofectamine LTX with Plus reagent was used to transfect pEZM02-lincRNA-VEGFR1 into HUVECs grown in 6 cm dishes. Three biologically distinct samples were used for each method. RNA was collected 24 hours post-transfection. Protein was collected 48 hours post-transfection.

Spheroid Sprouting Assay.

HUVECs isolated from female and male births (N=3) were transfected with 40 μM of Dicer substrate siRNA against lincRNA-VEGFR1 in 6 cm plates. Eight hours after transfection, the cells were trypsinized and suspended to generate 100 spheroids that were approximately 2250 cells per spheroid as previously described (Korff and Augustin 1999). Twenty-six hours after spheroid generation (34 h post-transfection), the 50 spheroids were collected and seeded on Matrigel (35% Matrigel, 35% Methocel and 30% FBS in endothelial medium) or collagen matrix (Korff and Augustin 1999). Sprouting was assayed 24 hours post-seeding in matrix. S.Core was used to quantify spheroid sprouting and statistical significance was assessed by student t-test.

Promoter Reporter Assays.

pGL2 constructs containing various promoters were used. pGL2-hFlt1(−1160 to +305), gift from Norihiko Takeda, was used to evaluate whether lincRNA-VEGFR1 interacted with the promoter of VEGFR1 (Takeda, Maemura et al. 2004). All reporter plasmids were transfected with either pEZM02-lincRNA-VEGFR1 or pCDNA-empty vector. The transfection experiments were carried out with lipofectamine LTX with Plus reagent on HUVEC lines (3 distinct biological samples). Dual-luciferase reporter assay from Promega was used and relative luminescence units (RLUs) were obtained on SpectraMax M5e. Firefly luciferase (FL) luminescence from pGL2 vectors was normalized against Renilla luciferase (RL) expressed by pRL-SV40 in order to normalize for transfection efficiency.

Tissue Samples and Cells.

Human umbilical vein endothelial cells were cultured as previously described (Marsden, Schappert et al. 1992; Flowers, Wang et al. 1995). Surgical specimens of glioma tumors were collected from the surgical suite at St. Michael's Hospital, following diagnostic confirmation by a neuropathologist. Tissues were obtained after patients' written consent under a protocol approved by the St. Michael's Hospital Research Ethics Board (REB) (REB #10-393). Tumors were dissociated into single cells as described in previous literature (Pollard, Yoshikawa et al. 2009).

Flow Cytometry.

GBM tumors (n=3) were dissociated as described above, and cells were stained with 1 μl anti-CD-144-PE (BD Biosciences) and 20 μL anti-CD-133-APC per 100 μl ice cold 0.5% BSA-1×HBSS (staining buffer). Cells were stained for 30 min then washed with staining buffer. Cells were filtered (40 μm) post-staining and suspended in 2 μl propidium iodide/mL (Sigma) and ice cold staining buffer. Samples were then processed through a fluorescence-activated cell sorting (FACS) Aria flow cytometer and data were analyzed using FlowJo software. FACS-sorted viable cell populations from GBM patients were used to extract total RNA using an RNeasy Kit (Qiagen). First-strand was synthesized from 35-50 ng of total RNA by Superscript VILO.

Differentiation of GBM Stem-Like Cells.

GliNS1 (previously referred to as G144ED) CD133+GBM stem-like cells were subjected to endothelial conditioned medium in order to obtain endothelial differentiation. GliNS1 line has been previously characterized to be tumorgenic, CD144⁻ and ˜95% CD133′ (Pollard, Yoshikawa et al. 2009). HUVECs were cultured in endothelial medium for 24 hours before collection of conditioned endothelial medium, the exact constitution of the endothelial medium used has been described previously (Fish, Matouk et al. 2007). Cells were treated with either standard stem cell medium or endothelial conditioned medium for 6 days prior to RNA collection and seeding on 3D Matrigel for 48 hours (N=4). S.Core was used to assess vascular network formation and statistical significance was assessed by student t-test. Expression of endothelial nitric oxide synthase was assessed by absolute quantification with standard curve by qRT-PCR.

RNA Interference in GBM Stem-Like Cells.

GliNS1 CD133+GBM stem-like cells were transfected with 40 μM Dicer substrate siRNA against lincRNA-VEGFR1 or Dicer substrate universal negative control (scrambled) using oligofectamine (per 6 cm plate). Endothelial differentiated GBM stem-like cells were cultured as previously described and transfected then seeded onto 3D Matrigel 16 hours post-transfection (N=4). Images were taken 72 hours post-transfection. The number of tubes was calculated by S.Core and subjected to student t-test analysis.

In Vivo Experiments.

Studies involving animals were in accordance with CCAC and approved by the Toronto Centre for Phenogenomics Animal Care Committee. 2.5 million GliNS1 cells were injected subcutaneously in the right lower flank of NOD-SCID mice. Tumor development was observed 3 to 4 weeks post-inoculation. Subcutaneous tumor size was measured using a caliper. The tumor volume in mm³ is calculated by previously established formula (Jensen, Jorgensen et al. 2008).

Nanoparticle Delivery of sIRNA.

Nanoparticle-based in vivo transfection reagent (Altogen Biosystems) was used to deliver 500 μM of either siRNA targeting LIVE (two independent dsiRNAs), negative control or Cy3-labeled negative control per injection. The nanoparticle reagent was prepared according to manufacturer's instructions. This polymer-based nanoparticle was previously characterized in vivo by the manufacturer. Nanoparticle-based RNAi was delivered through intratumoral injections every 48 hours. Injections were initiated when xenograft tumors reached approximately 1 cm³ in volume. Each animal received a total of 5 injections over 10 days.

Immunohistochemistry (IHC) and Immunofluorescence (IF).

GBM xenograft tumors were dissected and fixed in 10% buffered formalin phosphate for 24 hours at room temperature, rinsed in ethanol, paraffin embedded and sectioned (10 μm thickness) on cross section. The sections were de-paraffinized and rehydrated in a series of xylene, ethanol and PBS washes. IF: Antigen retrieval was performed by microwaving the sections in 10 mM sodium citrate, pH 6.0, 20 min at 100% power (720 Watt oven). Sections were rinsed and fibres were immunostained using pan anti-CD31 antibody (rabbit, 1:20 dilution; Abcam ab28364) with either anti-humanVEGFR1 (Flt1) (goat, 1:10 dilution; Calbiochem PC322L), anti-PCNA (1:1000; #2586 from cell signalling), anti-PDGFR-13 (1:1000 cell signaling), anti-NG2 (1:250; #5320 Milipore) or anti-SOX2 (1:1000; R&D) primary antibodies, followed by anti-rabbit Alexa 495 (1:250; Invitrogen) and biotinylated horse anti-goat antibodies (1:200, Vector Laboratory) for 1 hour at room temperature (RT). Sections were then washed 3×10 min in PBS and incubated for 1 hr at RT with Streptavidin-Alexa 594 (1:1000; Invitrogen) and DAPI or Hoechst (1:10,000, Sigma-Aldrich). Sections were washed 3×10 min with PBS and mounted in Dako mounting medium and imaged with a Zeiss 710 confocal microscope using ZEN software. IHC: Primary antibodies of anti-cleaved caspase 3 (1:1000; #9664 from cell signalling), anti-HIF-1α (1:10000; NB100-131 from Novus) and anti-HIF-2α (1:1000: NB100-122 from Novus) were used. Nuclei were counterstained with haematoxylin/eosin (H&E).

Histological Staining and Evaluation.

H&E and periodic acid-Schiff (PAS), as well as trichrome, staining protocols were performed by the Pathology core at the Centre for Modeling Human Disease in the Toronto Centre for Phenogenomics.

Fluorescein Anglography.

Each mouse with heterotrophic xenografts received either nanoparticle conjugated to control RNAi or RNAi against lincRNA-VEGFR1 as described previously, were anesthetized before tail vein injection with fluorescein. 2×10⁶ kDa FITC-Dextran (Sigma; FD2000S) was resuspended in PBS to a final concentration of 10 mg/ml and 1 ml was injected via tail-vein. 10 min post injection, mice were sacrificed and the tumours were placed in 4% paraformaldehyde for 3 hours before paraffin embedding and sectioning. Slides were visualized as described above.

Orthotopic Model and Alzet Pump Implantation.

2×10⁵ GLiNS1cells were injected in the right frontal lobe of NOD-SCID mice. AIZET brain infusion pumps (ALZET Brain Infusion Kit 3, cat #000885, and ALZET osmotic pumps cat#2002) were prepared and implanted 3 weeks after inoculation according to the manufacturer's protocol. 3000 μM/200 μL of dsiRNA against lincRNA-VEGFR1 or control dsiRNA were delivered through the cell injection site by pump for a 2-week time span. Animals were sacrificed at upon reaching an endpoint pre-defined by: persistent anorexia and dehydration that can't be alleviated or loss of body weight of more than 20%. Tissues were collected and fixed in 10% formalin for pathology analysis.

LIVE/DEAD Staining in GSCs.

GliNS1s were transfected with dsiRNA against LIVE as described above. Cy3-labeled dsiRNA was used to gauge transfection efficiency. Experiments where over 70% transfection efficiency was observed were subjected to staining protocol. 72 hr post-transfection cells were subjected to staining using LIVE/DEAD® Viability/Cytotoxicity Kit (Life technologies) according to manufacturer's instructions and visualized with Zeiss 710 confocal microscope. The dsiRNA identified as SEQ ID NO:4 in combination with SEQ ID NO:5 or lincRNA-VEGFR1 dsiRNA (I) was used for these experiments.

Over-Expression Lentiviral Vector.

LIVE cDNA was shuttled from pEZ-M02-lincRNA-VEGFR1 over-expression construct into pCDH-CMV-MCS-EF1-CopGFP vector under a CMV promoter with EF1 promoter driven expression of reporter CopGFP. Lentivector was custom packaged by System BioSciences (SBI™) at titer of 2.73×10⁸ IFU/ml. Low-LIVE expressing GSC line was chosen for the study. Using N=3 in each condition, GSC2012035 cells were transduced with either control lentivector or LIVE over-expression vector. Multiplicity of infection (MOI) of 10 was used in each condition. Cells were observed and collected at 72 hours post-transduction. GSCs were grown on 6 cm plates (4×10⁵ cells per plate), for an MOI of 10, LIVE lentivector=25 μl per plate, control lentivector=1.75 μl per plate. An infection absorption volume of 2 ml was used for each plate. Assays were performed within 76 hrs post-infection and lysates were collected. Cells were either flow sorted for pure GFP-expressing populations then implanted intracranially or RNA was extracted and used to assay for expression of LIVE and LIVE target genes via qRT-PCR with standard curve and exogenous luciferase normalization.

RNA Pull-down Mass Spectrometry. T7 promoter LIVE construct (GeneCopoeia™) was linearized and gel purified (QIAquick) Gel Extraction Kit, Qiagen). The linearized transcript was then used for in vitro transcription (MEGAscript® Kit, Life Technologies™) and transcriptional products were purified using MEGAclear® kit (Life Technologies™). Purified transcriptional products were then analyzed on Bioanalyzer (Agilent Technologies Inc.) to validate the transcript size and purity. Nuclear extracts were isolated from GLiNS1 using NE-PER™ nuclear and cytoplasmic extraction reagents (Thermo Scientific™) and Bradford protein assay was performed to calculate protein concentration. Nuclear extracts were dialyzed on the day of use (Slide-A-Lyzer MINI Dialysis Units, Thermo Scientific™) in 1 L BupH™ Carbonate-Bicarbonate buffer (Thermo Scientific™) for 75 min at 4° C. Pierce™ Magnetic RNA-Protein Pull-Down Kit (Thermo Scientific™) was used with androgen receptor (AR) RNA as a positive control and negative control poly(A)₂₅ RNA was used in parallel with two concentrations of LIVE RNA. Final eluents were sent for MS/MS at the SPARC Bioscience Centre at the Hospital for Sick Children.

Q-Exactive Method.

The peptides were loaded onto a 50 cm×75 μm ID column with RSLC 2 μm C18packing material (EASY-Spray, Thermo-Fisher, Odense, Denmark) with an integrated emitter. The peptides were eluted into a Q-Exactive hybrid mass spectrometer (Thermo-Fisher, San Jose, Calif.) using an Easy-Spray nLC 1000 chromatography system (Thermo-Fisher, Odense Denmark) with a 1 hr. gradient from 0% to 35% acetonitrile in 0.1% formic acid. The mass spectrometer was operated in a data-dependent mode with 1 MS followed by 10 MS/MS spectra. The MS was acquired with a resolution of 70,000 FWHM, a target of 1×10⁶ ions and a maximum scan time of 120 ms. The MS/MS scans were acquired with a resolution of 17,500 FWHM, a target of 1×10⁶ ions and a maximum scan time of 120 ms using a relative collision energy of 27%. A fixed first mass of 80 Da and a dynamic exclusion time of 15 seconds was used for the MS/MS scans. The raw data file was acquired with XCaibur 2.2 and processed with Proteome Discover 1.4 (Thermo-Fisher, San Jose, Calif.). The peptide identifications were imported into Scaffold 4 (Proteome Software, Portland, Oreg.) for spectral counting and GO annotation.

RNA-binding Protein Immunoprecipitation (RIP). Lysates extracted from GLiNS1 cells were subjected to Magna RIP™ (EMD Millipore) where the assay was carried out according to manufacturer's protocol. 5 μg of anti-RNA Helicase antibody (Abcam® ab26271) and anti-PARP antibody (Abcam® ab137653) were used per 100 g of lysate. The RNA isolates were synthesized into first strand using Superscript VILO (Invitrogen) and RNA levels were measured via qRT-PCR using Viia7 (Life Technologies) using absolute quantification standard curve.

RNA Interference in GBM Stem-Like Cells.

GliNS1 CD133+GBM stem-like cells were cultured in antibiotic-free-standard medium and transfected with 40 μM Dicer substrate siRNA against lincRNA-VEGFR1, other target siRNAs or Dicer substrate universal negative control (scrambled) using oligofectamine (per 6 cm plate). Protein was collected 72 hr post-transfection. Western blotting analysis was performed as previously described.

PARP1 Inhibition in GSCs.

BYK204165 (Sigma, cat.B3188) was resuspended in DMSO according to manufacturer's instructions. GSCs were cultured as described above and kept in antibiotic-free medium for 24 hr prior to transfection with Oligofectamine according to manufacturer's instructions (Invitrogen, Life Technologies). Cells were treated with BYK204165 at final concentration of 3 μM for 2 hours prior to protein and RNA extraction. Protein and RNA extraction, qRT-PCR and western blots were performed as described.

GSC RFP Reporter Line.

GliNS1 were transduced by Cignal Lenti RFP Reporter (Qiagen cat#336891) at an MOI=1. Puromycin selection was used to isolate stable integration events. Stably transduced GSCs were intracranially injected at 100,000 cells/mice to generate orthotopic xenografts. All mice were sacrificed at a pre-defined endpoint. Mouse brain sections were IF stained as described above.

### Results

VEGF-A₁₆₅ Elicits Distinct lncRNA Profiles in Human Endothelial Cells

To interrogate dynamic changes in the steady-state lncRNA profile in human endothelial cells, four distinct biological samples of human umbilical vein endothelial cells (HUVECs) were subjected to treatment with or without VEGF-A₁₆₅ in either a 2D monolayer or 3D network on Matrigel. RNA was collected and hybridized to the Human LncRNA Array V2 (Arraystar).

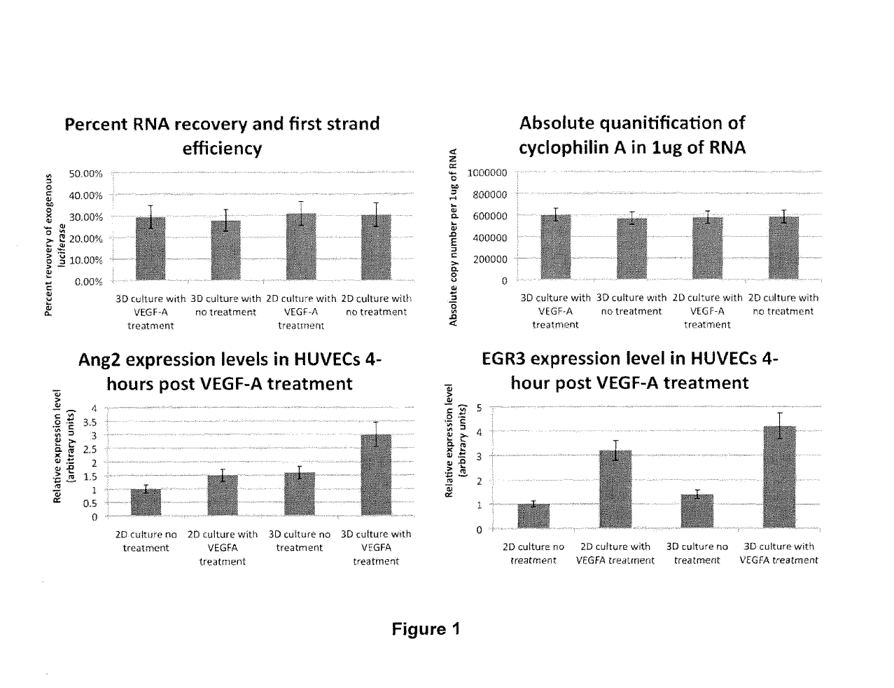

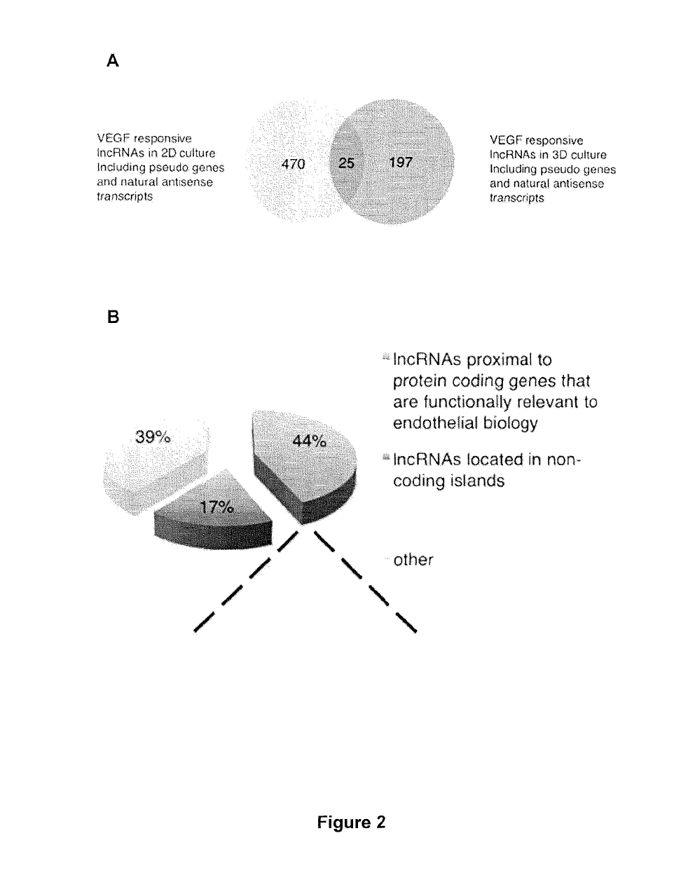

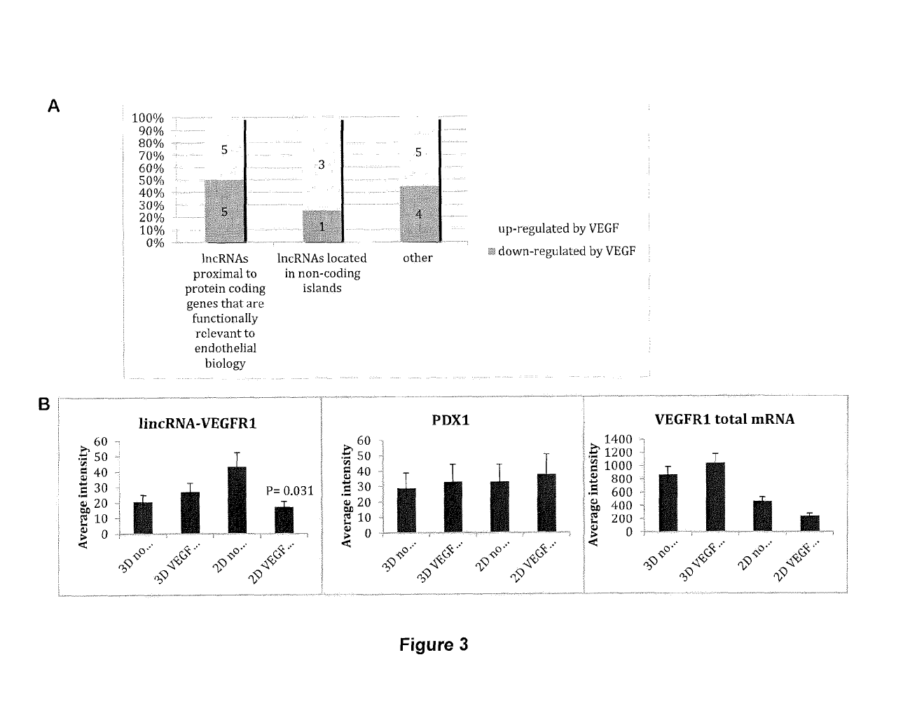

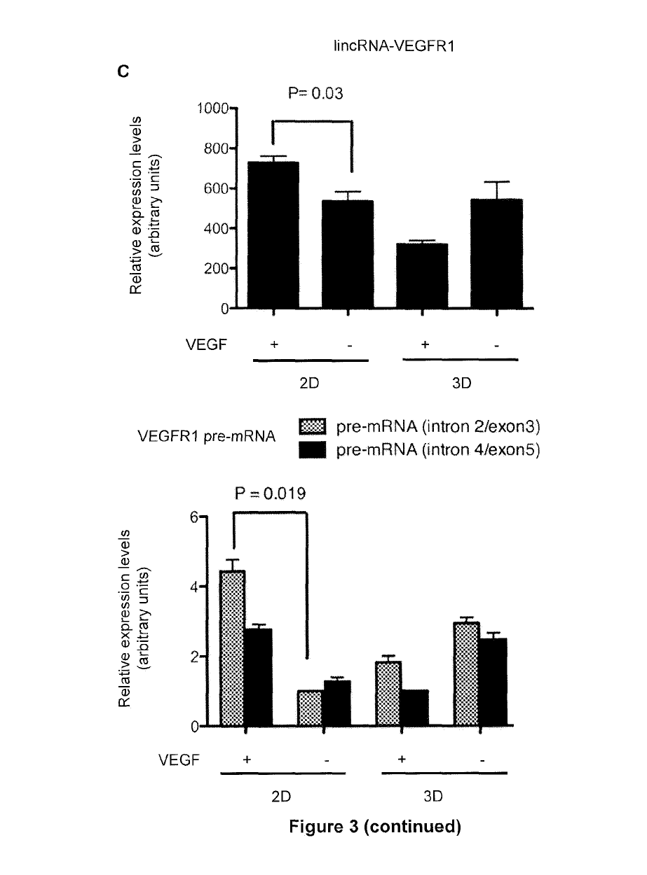

VEGF-A elicited distinct coding mRNA profiles in each culture condition, consistent with prior descriptions (FIG. 1) (Abe and Sato 2001; Wary, Thakker et al. 2003). The lncRNA profile of all four groups was then compared to identify VEGF-A responsive lncRNAs. VEGF-A elicited a distinct lncRNA signature in HUVECs cultured in 2D and 3D cultures, with minimal overlap between the two morphological states (FIG. 2A). Ensembl and UCSC were used to categorize each lncRNA target and obtain transcript sequences. Natural anti-sense transcripts and pseudogenes were excluded. From the 33,045 lncRNA transcript probe sets screened, the top 23 lincRNA candidates were identified based on greatest fold-change and smallest p-value. The genomic location of 44% of the top candidates was within 500 kb of a protein encoding gene functionally relevant to endothelial biology, such as GATA2 and angiopoietin 4, a phenomenon that has been observed previously in other organ systems (FIGS. 2B and 3A) (Cabili, Trapnell et al. 2011). The expression profile of the top 5 candidates from this group was validated using quantitative real-time PCR (qRT-PCR). One large intergenic non-coding RNA, located 452,489 bp downstream of VEGFR1 (also known as FLT1), was selected for further study, which was termed lincRNA-VEGFR (LIVE) (FIGS. 3 B,C).

Identification of lincRNA-VEGFR1 (UVE) and its Effects on Endothelial Biology and Angiogenesis

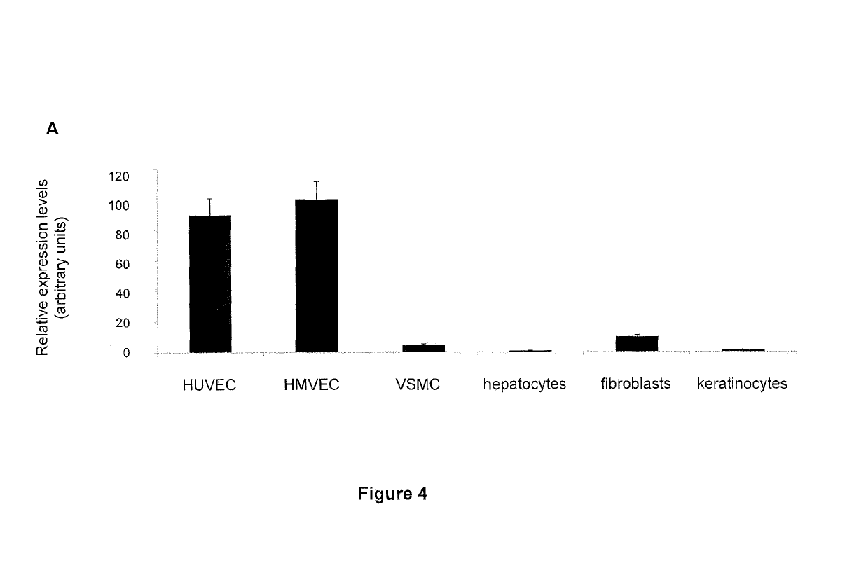

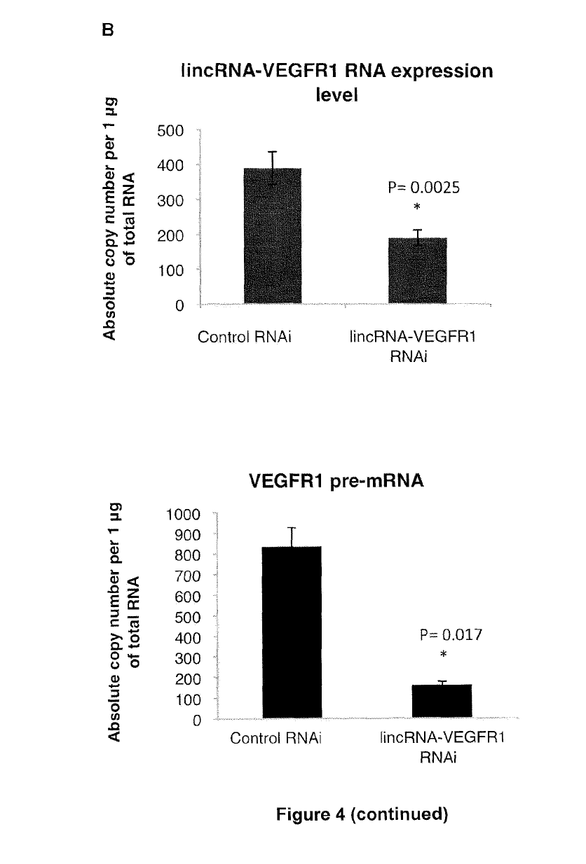

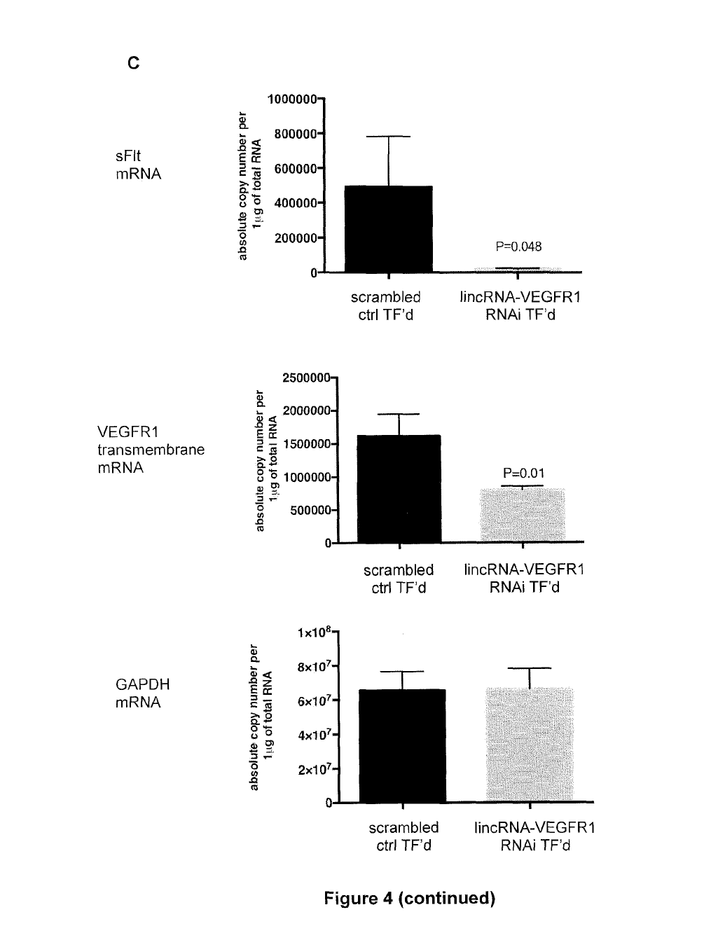

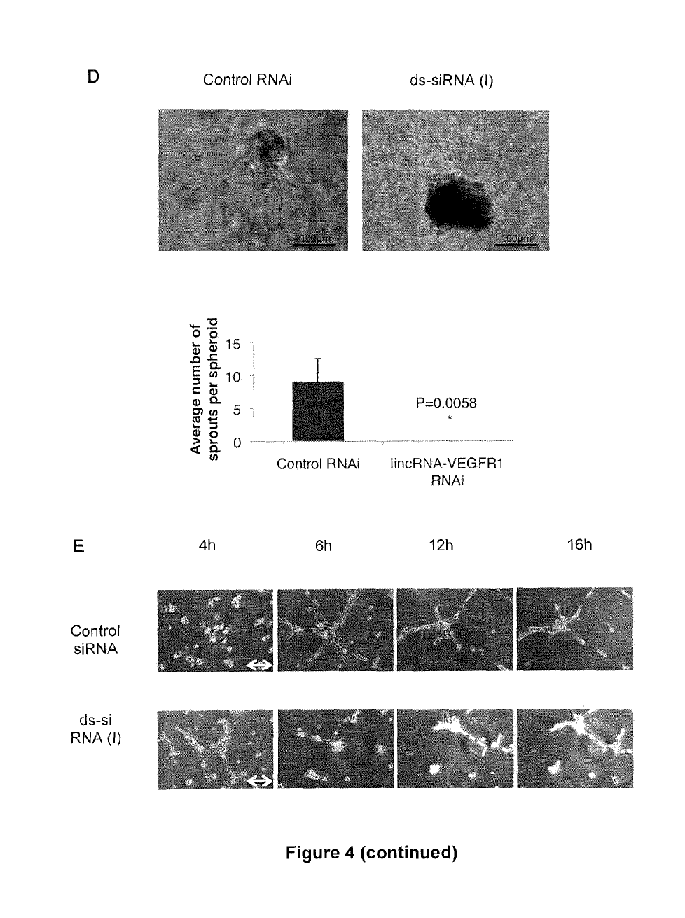

Although the gene encoding LIVE shares genomic sequence homology across higher order primate species, no annotated ESTs were found in deposited databases. The txCDsPredict score for LIVE is 158.00, suggesting that its protein coding potential is limited, and LIVE lack an in-frame start or stop codon. The gene encoding LIVE spans 18,694 bp and includes 3 exons. The processed transcript is 1751 bp as determined by 5′RACE, EST and RNA-seq data from ENCODE/Ensembl. In a panel of 6 human primary cell types, LIVE was found to be enriched in endothelial cells (FIG. 4A).

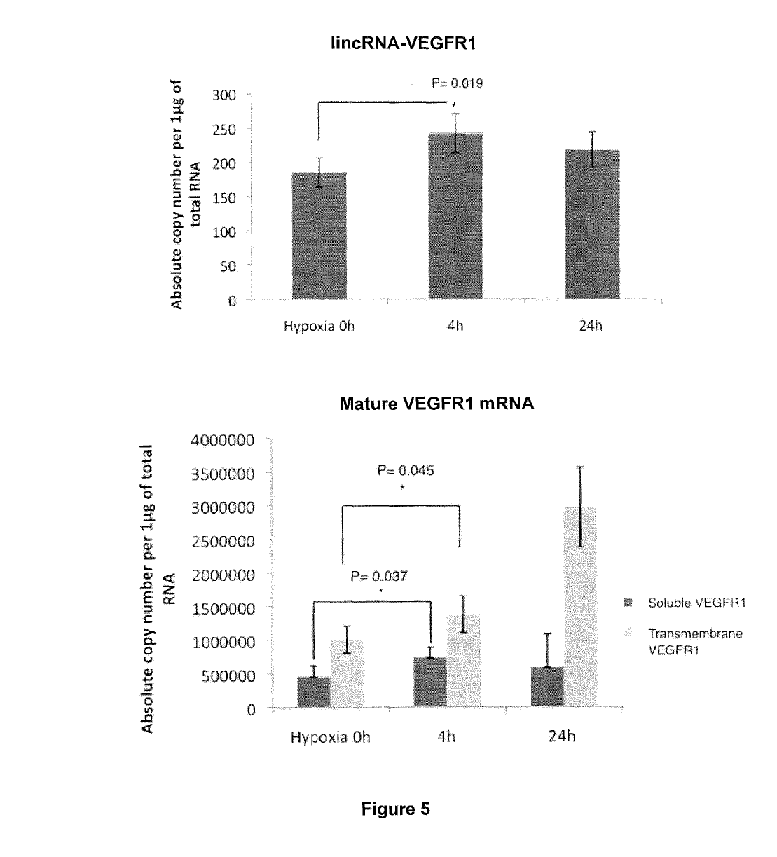

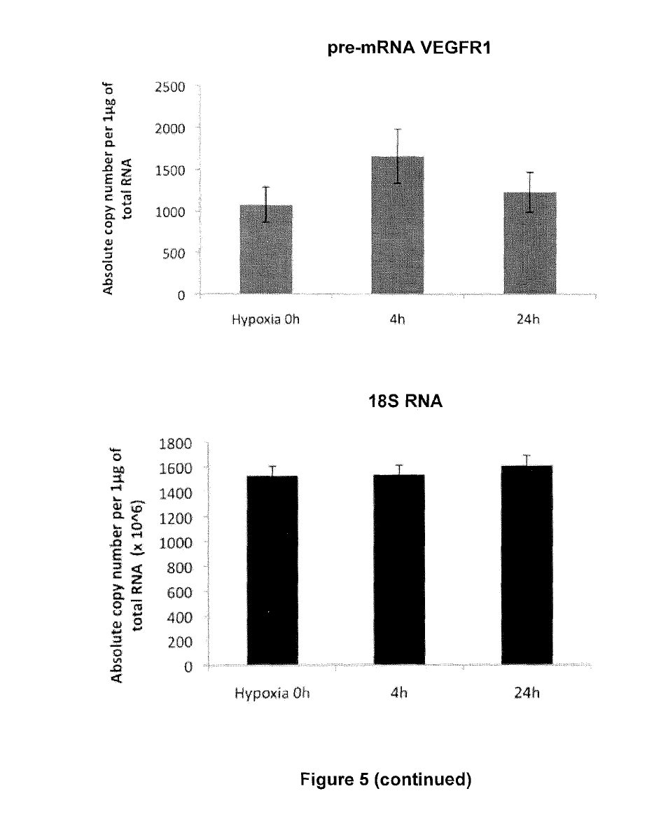

The VEGFR1 gene encodes two distinct functional variants: a trans-membrane receptor and a soluble variant that acts as an inhibitory decoy receptor. Both are crucial to angiogenesis (Ferrara, Gerber et al. 2003; Chappell, Taylor et al. 2009). VEGFR1 and soluble VEGFR1 (sFlt) have been shown to be crucial for angiogenic sprouting and vascular network formation and to underlie pericyte function (Boeckel, Guarani et al. 2011; Jin, Sison et al. 2012). As alternative splicing of VEGFR1 pre-mRNA is important in the regulation of VEGF activity, VEGFR1 pre-mRNA levels were studied as an indicator of overall transcription of VEGFR1 gene in the context of VEGF treatment (He, Smith et al. 1999). In addition, the steady state levels of mature mRNA products are dependent on transcriptional input as well as post-transcriptional regulation and RNA stability. To assess transcriptional input, VEGFR1 pre-mRNA was measured with primer sets that crossed intron-exon boundaries, which is reflective of overall transcription of both VEGFR1 transcriptional products (FIG. 3C). LIVE levels did not correlate with the expression profile of mature VEGFR1 gene mRNA products, but did correlate with VEGFR1 pre-mRNA products (FIGS. 3 B,C). To examine whether LIVE is responsive to other environmental stimuli relevant to endothelial biology, HUVECs were subjected to hypoxia for 4 and 24 hr, which has been previously found to increase VEGF transcript abundance and affect endothelial biology (White, Carroll et al. 1995; Claffey, Shih et al. 1998; Fischer, Clauss et al. 1999). LIVE expression increased significantly after 4 hours of hypoxia, as did VEGFR1 pre-mRNA expression but not mature VEGFR1 mRNA expression (FIG. 5).

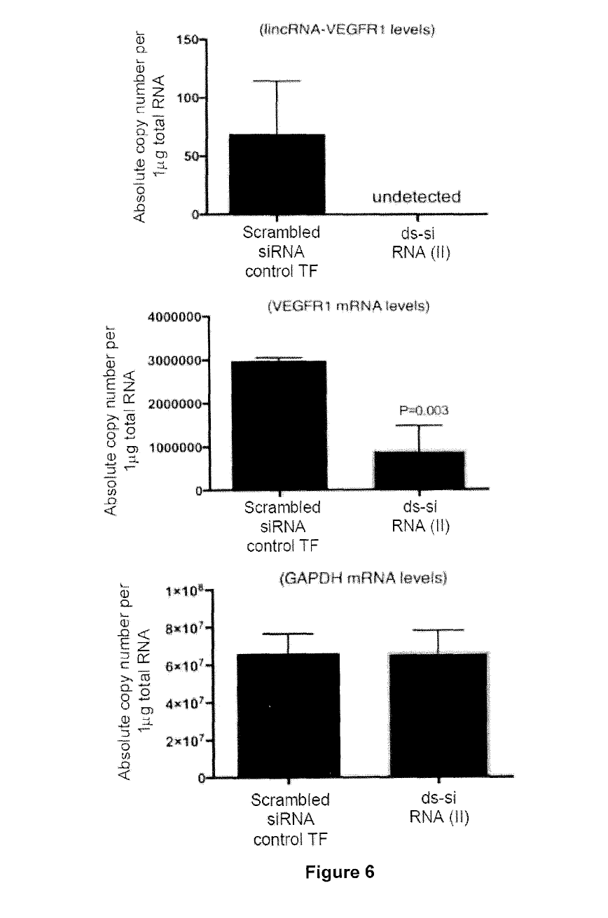

To determine if LIVE effects VEGFR1 transcription and endothelial cell function, Dicer substrate siRNA (dsiRNA (I), 27-mer) was used to knockdown LIVE in HUVECs. As LIVE transcript decreased in abundance, VEGFR1 pre-mRNA decreased by 4-fold, soluble VEGFR1 (sFIt) mature mRNA decreased by 4-fold and transmembrane VEGFR1 mRNA decreased by over 2-fold (FIGS. 4 B,C). Downregulation of VEGFR1 mRNA by LIVE knockdown was validated with a second siRNA (dsiRNA (II)) (FIG. 6). Thus, two dsiRNAs produced consistent knockdown under high transfection efficiency (FIG. 4B and Table 1).

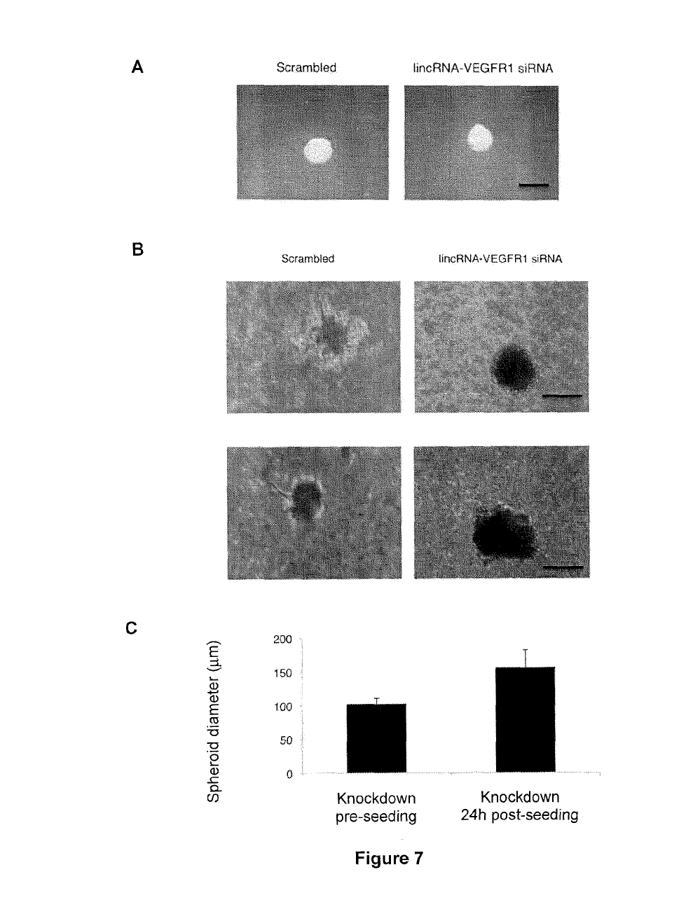

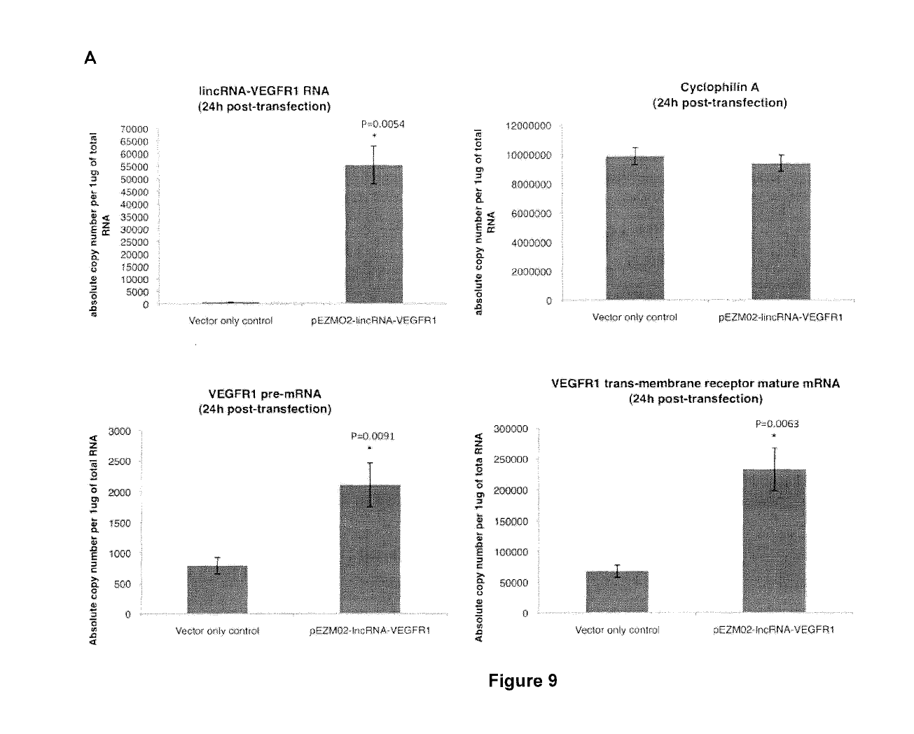

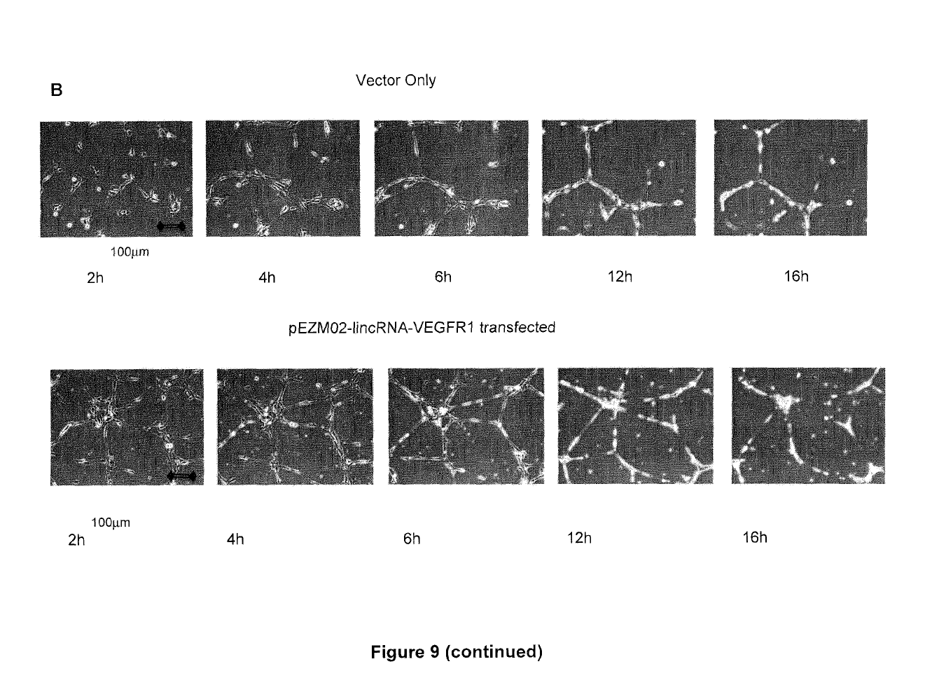

To interrogate whether LIVE functionally participates in endothelial biology, its effects on vascular formation were studied using two in vitro assay systems. First, HUVECs were transfected with control or LIVE siRNA and cultured as spheroids in collagen matrix (FIG. 7A). Spheroids formed from HUVECs transfected with control siRNA formed angiogenic sprouts of varying length and number, whereas spheroids derived from HUVECs transfected with LIVE dsiRNA increased in size but did not produce sprouts (FIGS. 4D, E and FIG. 7). Spheroid-sprouting assays were also performed in Matrigel and revealed similar findings (FIG. 7B). Second, time-lapse images were taken over 16-hour time course to evaluate the impact of LIVE knockdown on vascular network formation on Matrigel (FIG. 9B). The process of vascular network formation of HUVECs on Matrigel has been described as occurring though five stages: aggregation (1 hr post-seeding), spreading (2 hr), elongation (3-4 hr), plexus stabilization (4-7 hr) and plexus reorganization (8-12 hr) (Parsa, Upadhyay et al. 2011). Cells transfected with dsiRNA against LIVE were unable to form stable networks on Matrigel compared to cells transfected by control dsiRNA (FIG. 9B).

To determine the effect of LIVE over-expression on vascular network formation, HUVECs were transfected with a vector engineered to result in overexpression of LIVE and then plated on Matrigel. Compared to empty vector-transfected control, LIVE over-expressing cells underwent accelerated plexus stabilization at 2-hr post-seeding and formed significantly more networks (FIG. 9A).

LIVE is Highly Expressed in Human Glioblastoma and is Enriched in CD133⁺ Glioma Stem Cells and CD133⁺CD144⁺ Neoplastic Endothelial Progenitor Cells

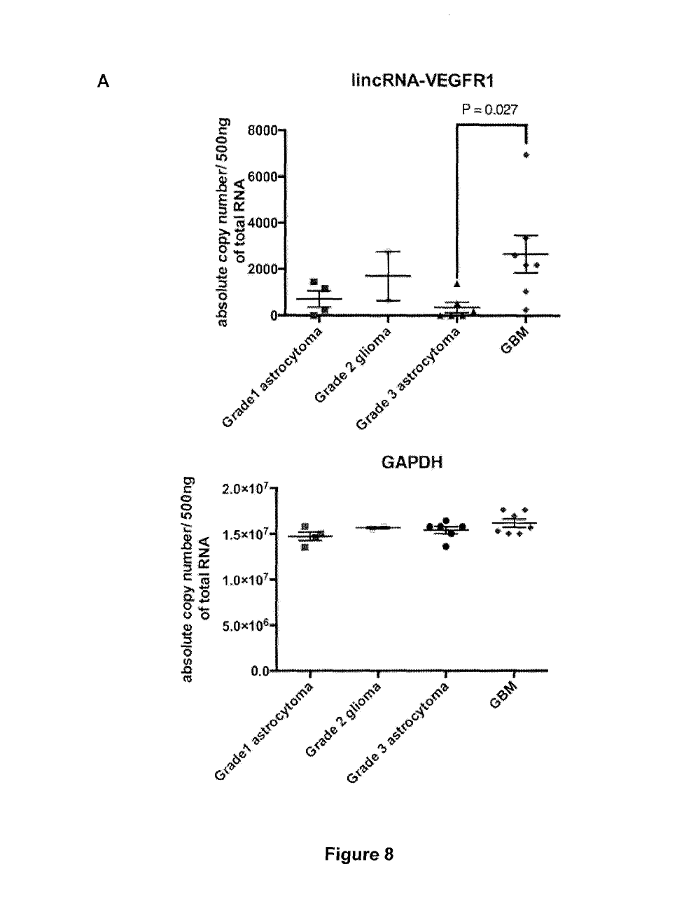

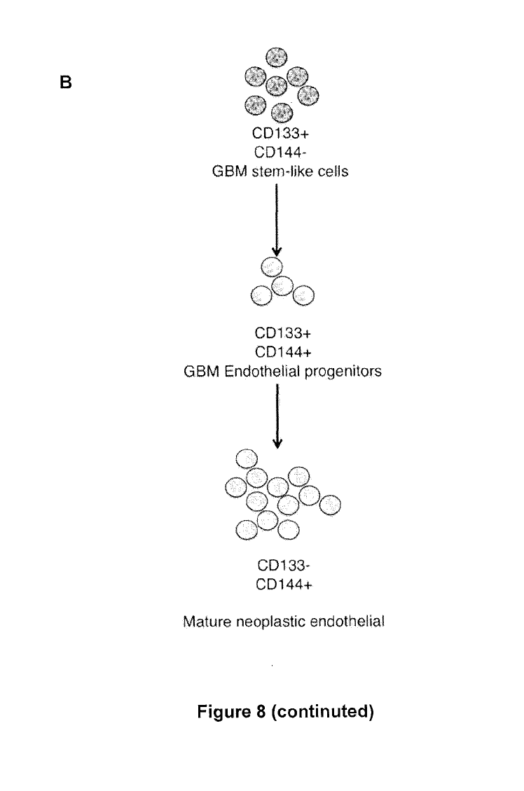

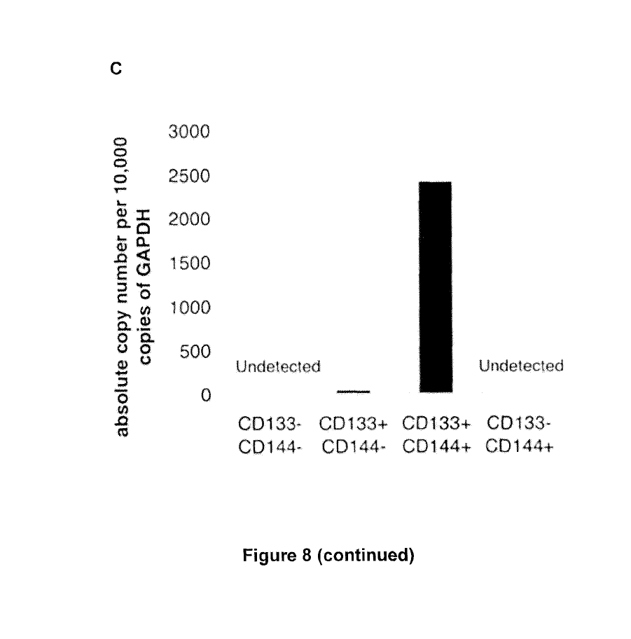

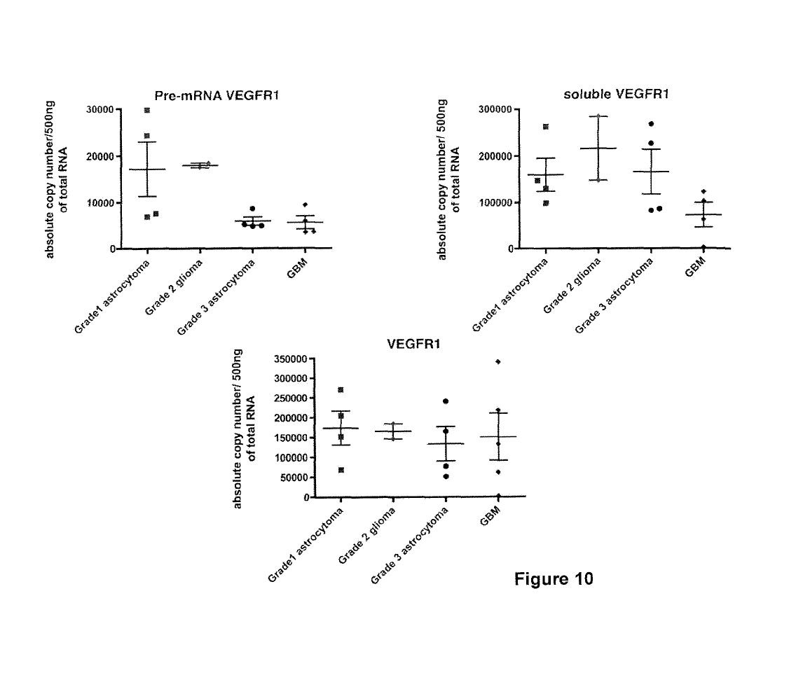

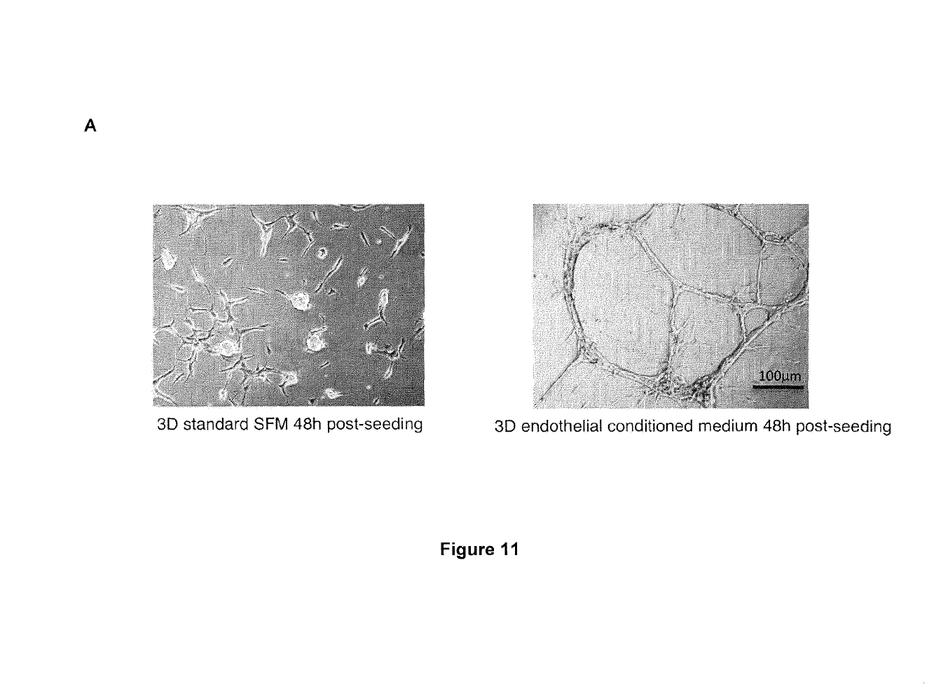

Glioblastoma is the most common primary brain tumour in adults. Despite the employment of aggressive, multi-modality therapy involving all of surgery, radiation and chemotherapy, median survival following diagnosis with glioblastoma remains only 14 months (Stupp, Mason et al. 2005). One of the features distinguishing glioblastoma from lower grade gliomas is the presence of microvascular proliferation. In a panel of 19 gliomas, LIVE was found to be expressed in gliomas of all grades but expressed at the highest levels in glioblastoma (FIG. 8A). VEGFR1 transcriptional products were found in gliomas of all grades (FIG. 10).

In addition to their role in tumour initiation and propagation (Singh, Hawkins et al. 2004), glioma stem cells (GSCs) have recently been found to give rise to a CD133⁺CD144⁺ population capable of maturation into endothelial cells and pericytes (FIG. 8B) (Ricci-Vitiani, Pallini et al. 2010; Wang, Chadalavada et al. 2010; Cheng, Huang et al. 2013). To interrogate the expression profile of LIVE within glioblastoma, three acutely dissociated human glioblastoma specimens were subjected to FACS to sort for CD133+ and CD144+ expression. LIVE expression levels were measured in four isolated cell populations. LIVE was enriched in the CD133⁺CD144⁻ (GSC) and CD133⁺CD144⁺ (neoplastic endothelial progenitor) populations. LIVE was undetectable in the CD133⁻CD144⁺ (mature endothelial cell) population, and very low levels of expression were identified in the CD133⁻CD144⁻ (non stem-like) fraction (FIG. 8C).

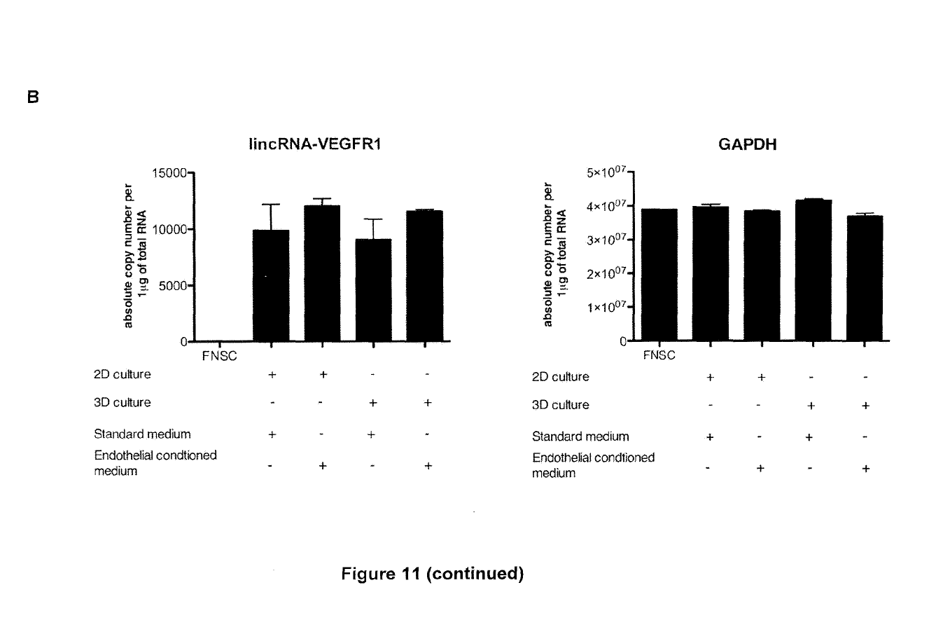

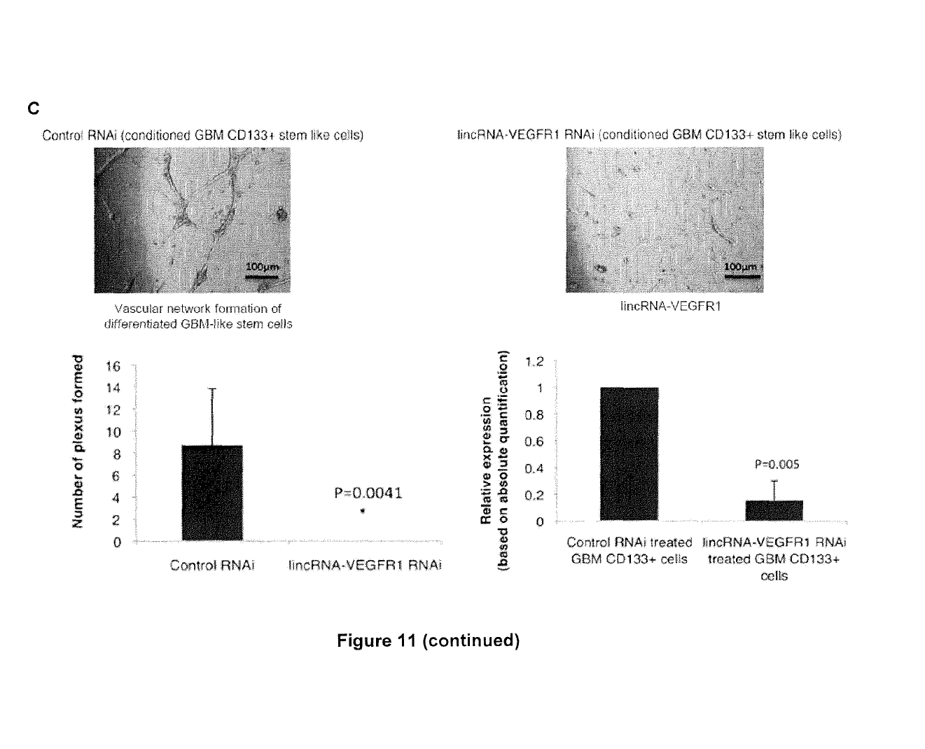

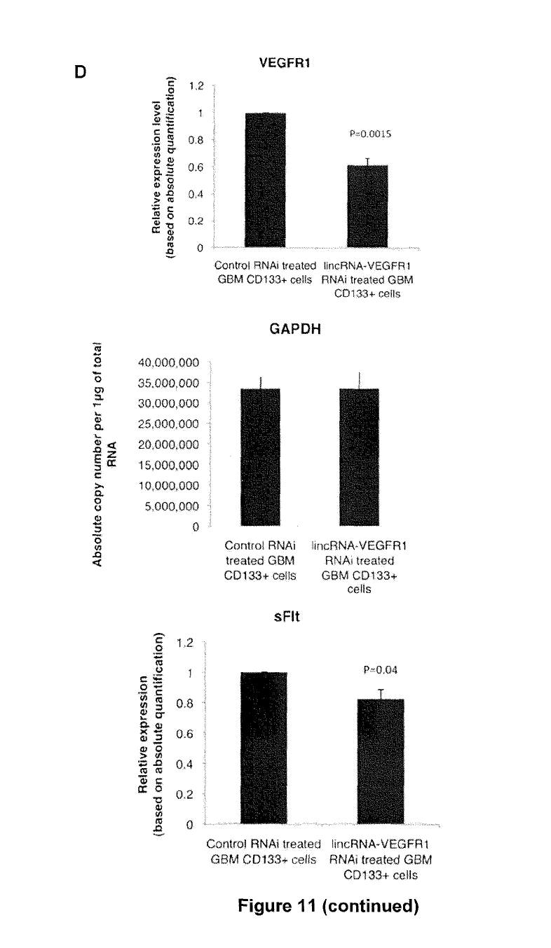

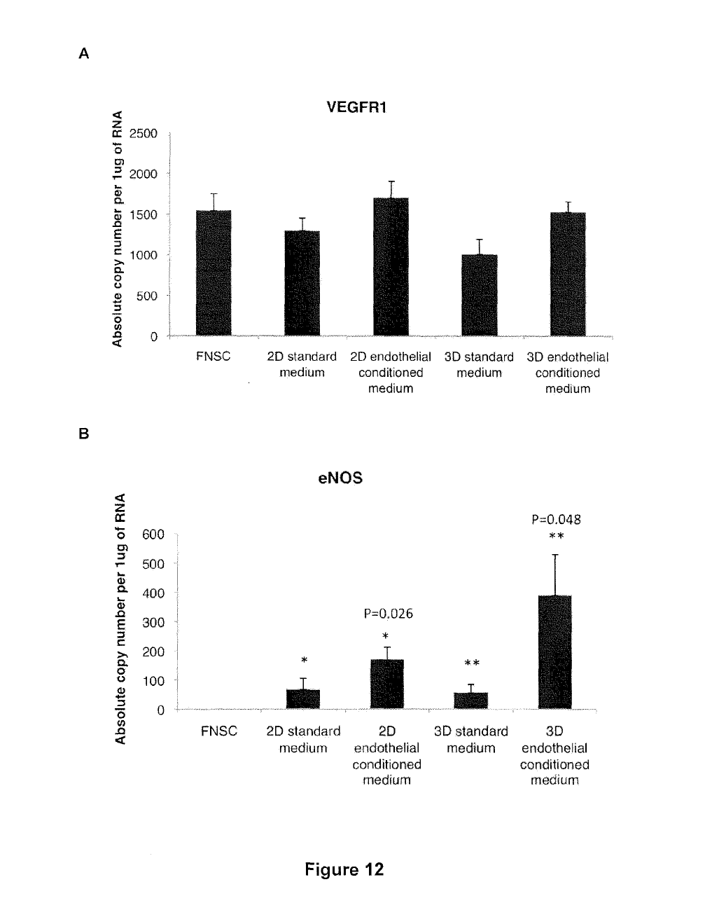

To assess the role of LIVE expression in angiogenesis in glioblastoma, CD133⁺ GSCs were cultured in either serum-free medium with growth factors to maintain a stem-like state or endothelial cell-conditioned medium to induce endothelial differentiation (Pollard, Yoshikawa et al. 2009; Ricci-Vitiani, Pallini et al. 2010; Wang, Chadalavada et al. 2010; Soda, Marumoto et al. 2011). Consistent with previous reports, GSCs adopted an endothelial cell phenotype in endothelial-conditioned medium (FIGS. 11A and 12) (Ricci-Vitiani, Pallini et al. 2010). LIVE expression remained high in both CD133⁺ glioma cells cultured in stem-cell media and following endothelial differentiation (FIG. 11B). Endothelial-differentiated glioma cells expressed significantly higher levels of nitric oxide synthase (eNOS) and formed highly structured vascular networks when seeded on Matrigel (FIG. 11A and FIG. 12). Knockdown of LIVE in CD133⁺ cells by transfection with Dicer substrate LIVE siRNA led to a decrease in transcript abundance of VEGFR1 and soluble VEGFR1 (sFlt) mature mRNA, and inhibited plexus formation on Matrigel compared to control siRNA (FIGS. 11 C,D).

### Intratumoural Nanoparticle-Mediated Delivery of RNAi Against LIVE Decreases Tumour Volume in a Glioblastoma Xenograft Model

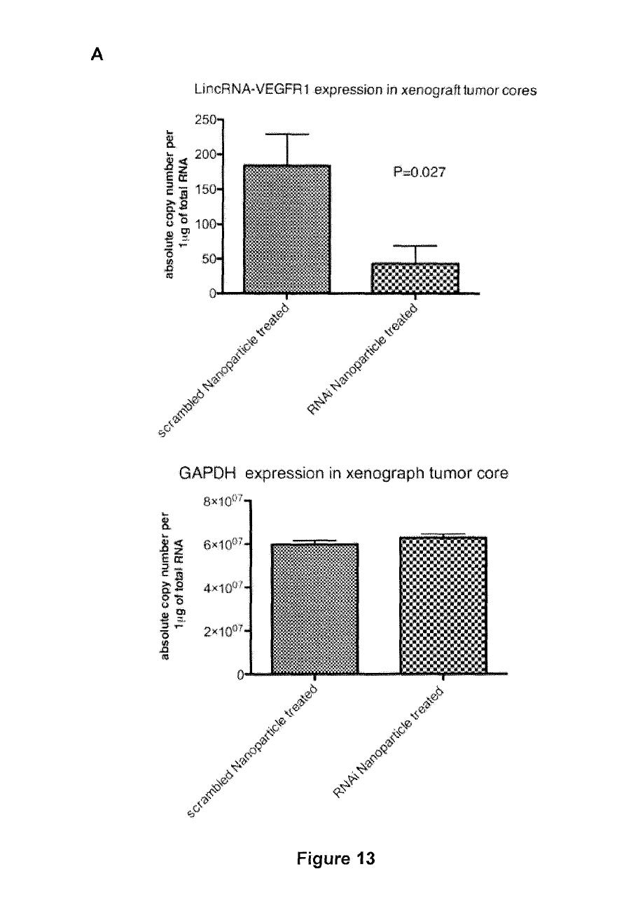

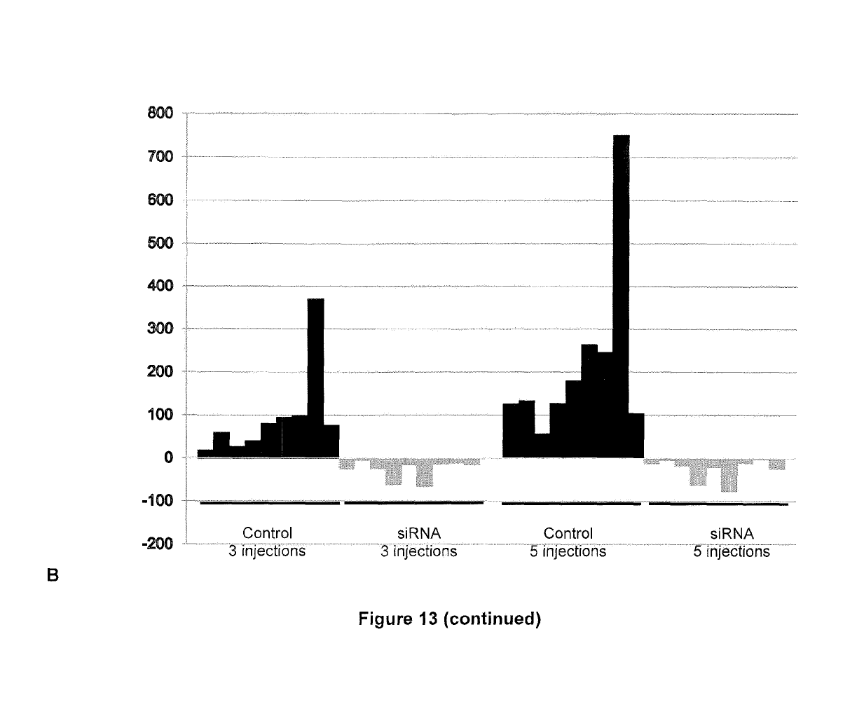

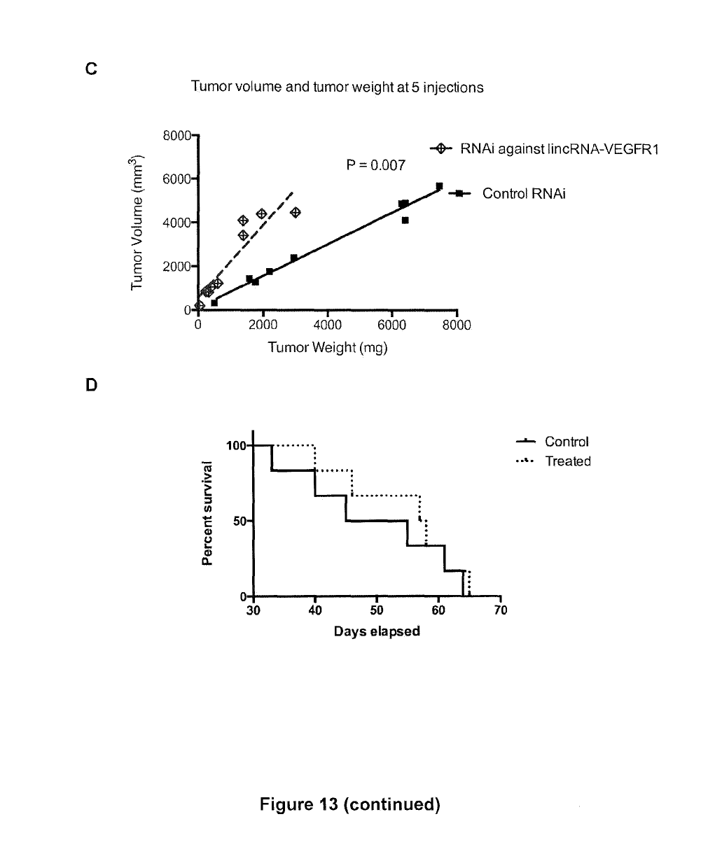

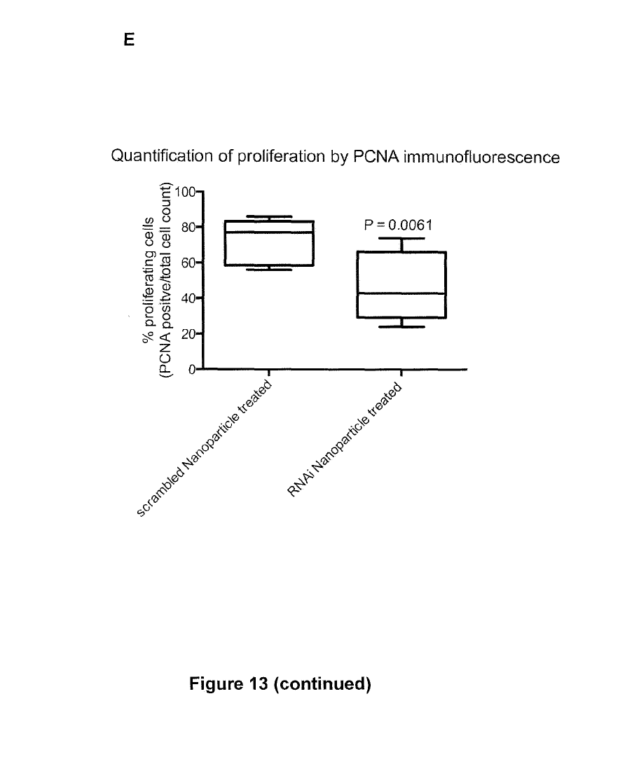

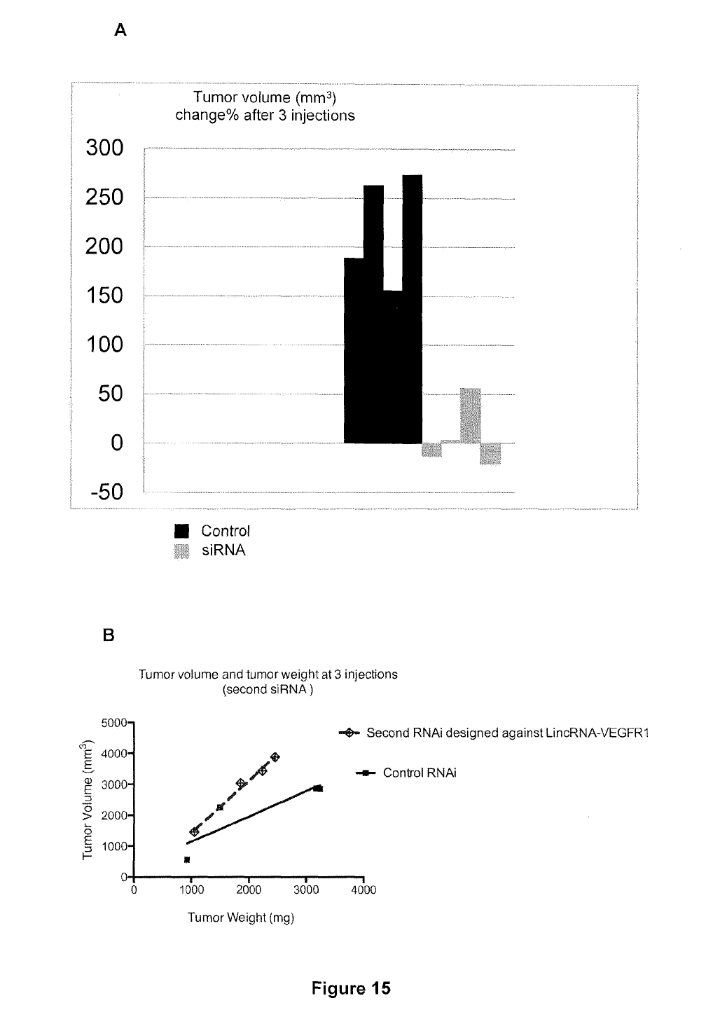

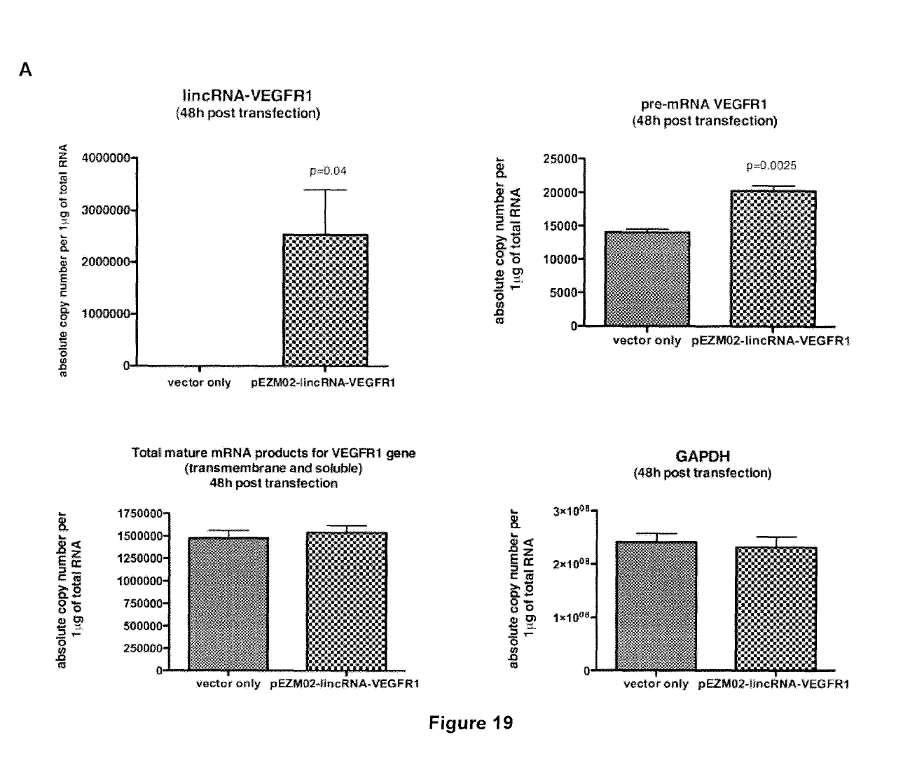

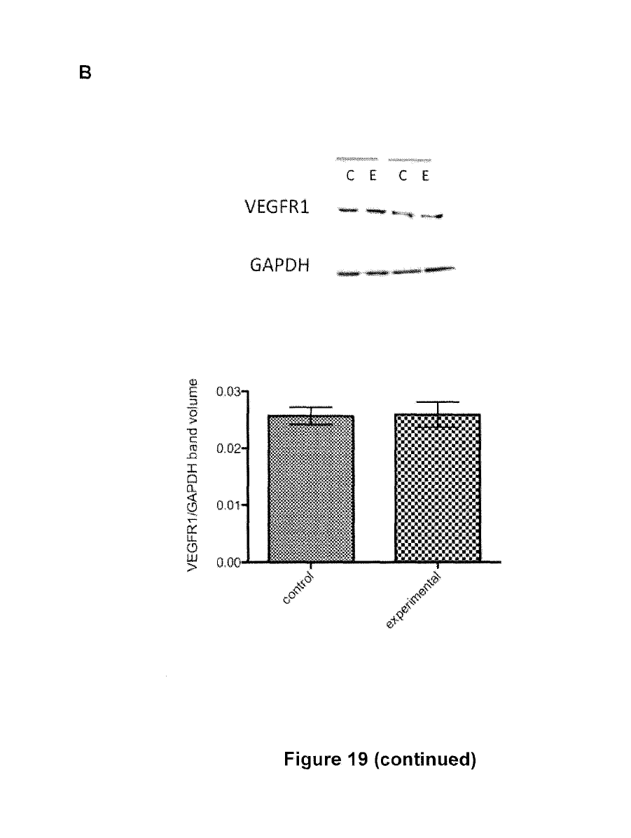

To evaluate the role of LIVE within the glioblastoma microenvironment, knockdown studies were performed using a nanoparticle-mediated siRNA delivery system targeting LIVE in a glioblastoma xenograft model. Nanoparticles carrying Dicer-substrate siRNA against LIVE or scrambled (negative) control were delivered by intratumoural injection into subcutaneous xenograft tumours in NOD-SCID mice (N=9 in each group). Injection of siRNA against LIVE resulted in effective knockdown of LIVE expression in xenograft tumour cores compared to controls (P=0.027, 74%; FIG. 13A). LIVE knockdown resulted in a decrease in tumour volume and weight compared to control in all heterotopic tumours studied (FIG. 13B,C and FIG. 19). Although overall survival was unchanged, tumour progression was slowed with osmotic pump administration of dsiRNAs against LIVE in an intracranial xenograft model (N=5 in each group; FIG. 13D). Similar results were found with employment of a second dsiRNA (dsiRNA (II)) against LIVE (FIG. 15). Compared to control tumours, LIVE knockdown tumours showed increased intratumoural necrosis (P=0.038; FIG. 11E) and decreased cell proliferation, as determined by staining for the cell proliferation marker, PCNA (47%, P=0.0061; FIG. 13E), both of which could account for the slowing of tumour progression seen with LIVE knockdown.

### Knockdown of LIVE Decreases Vascular Density and Pericyte Coverage in Glioblastoma

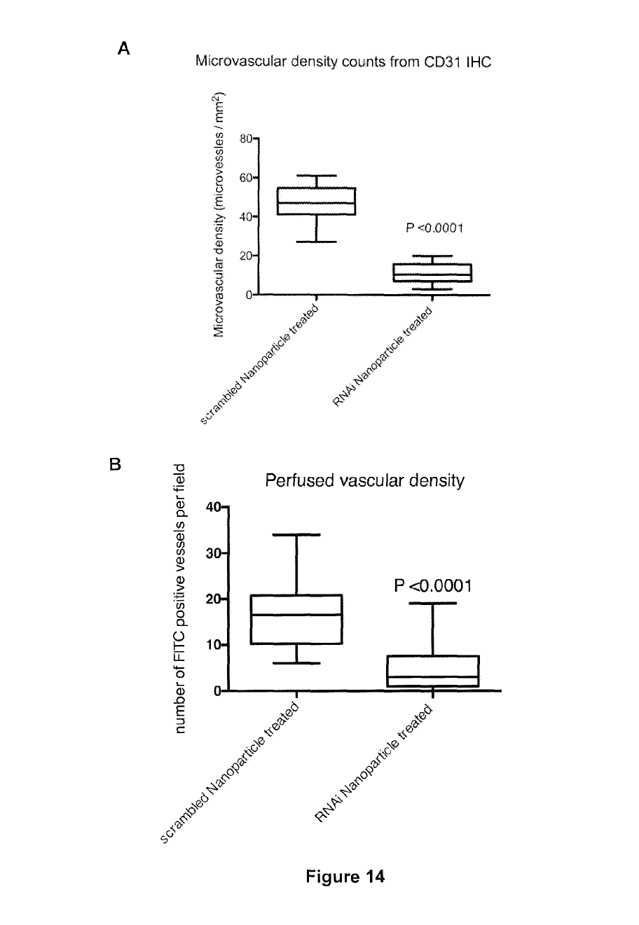

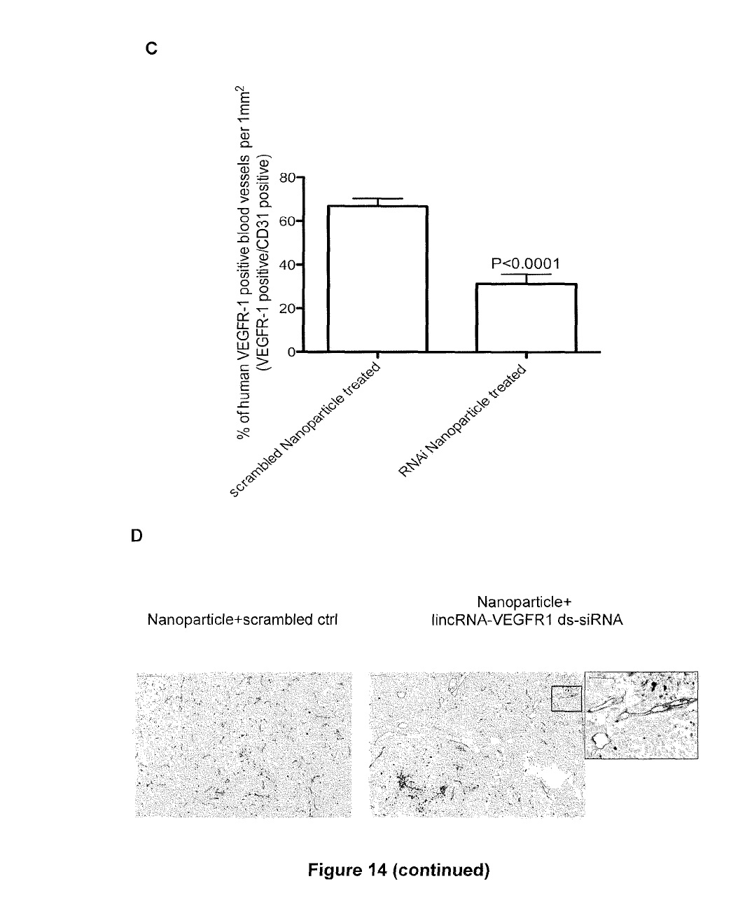

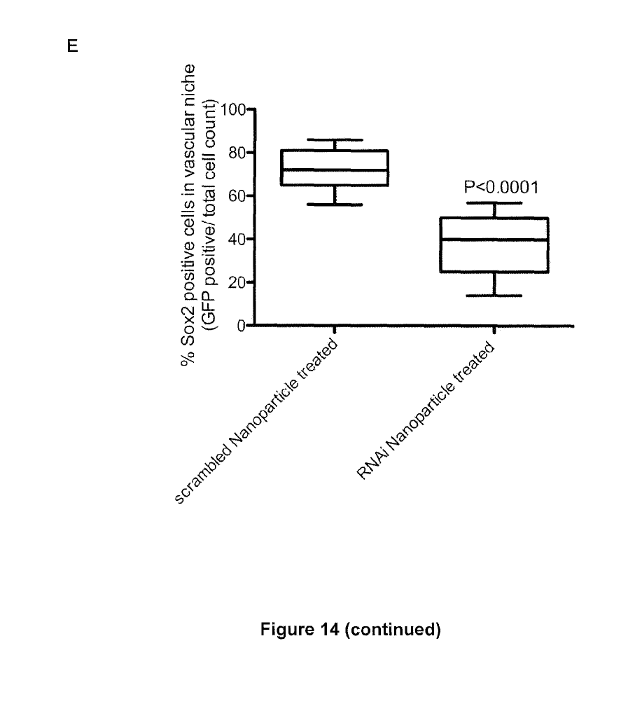

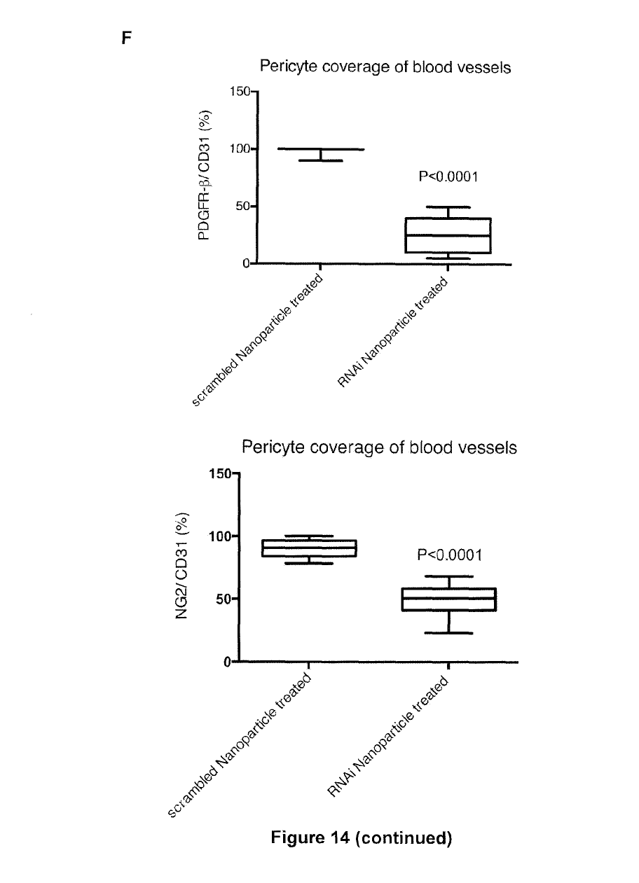

To determine the changes underlying the increase in necrosis and decrease in cell proliferation seen following LIVE knockdown in glioblastoma, immunohistochemistry (IHC) for CD31 was used to evaluate microvascular density (MVD) in LIVE knockdown tumours compared to control. MVD was decreased by 5-fold in LIVE-targeted tumours compared to control tumours (P<0.001; FIGS. 14 A,B). Using high molecular weight FITC-dextran fluorescein angiography, the number of functional vessels was then quantified in treated and control tumours. LIVE knockdown resulted in a 72% decrease in functional intratumoural blood vessels compared to control (P<0.0001; FIG. 14C).

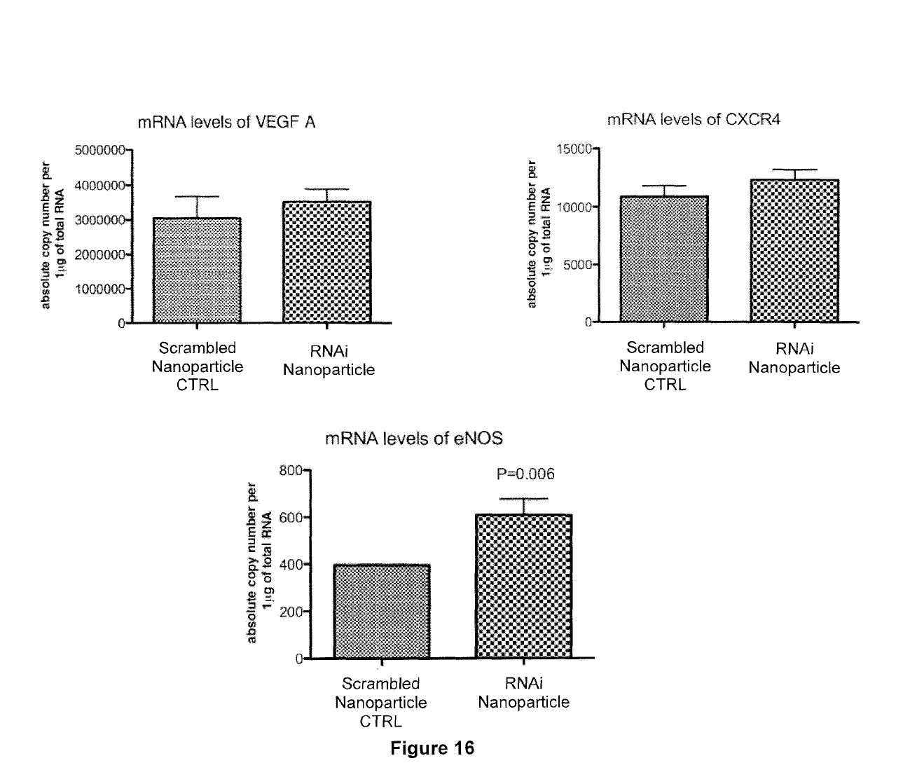

The present inventors then sought to ascertain the etiology of the decrease in MVD associated with RNAi-based targeting of LIVE in glioblastoma. Using antibodies against human-specific VEGFR1 and human- and mouse-specific CD31 (FIG. 14D), IHC of mouse xenograft tumours showed that LIVE knockdown resulted in a substantial decrease in human VEGFR1-positive blood vessels compared to tumours treated with scrambled control (53%, P<0.0001; FIG. 14D). No significant differences were found in HIF-1α and HIF-2α expression, nor in HIF-dependent (VEGF-A, CXCR4) or—independent (eNOS) mRNA transcripts known to be affected by hypoxia between the treatment group and control (FIG. 16).

Using cleaved caspase 3 to evaluate for apoptosis, large apoptotic clusters were observed near CD31⁺ microvessels in LIVE-targeted tumours when compared to control tumours. Co-immunofluorescence (Co-IF) staining for Sox2 and CD31 showed a decrease in the perivascular Sox2-positive glioma cell population (54%, P<0.0001; FIG. 14E). Moreover, co-IF staining with CD31 and two independent pericyte markers, PDGFR-β and NG2, demonstrated a decrease in pericyte coverage of vessels in LIVE knockdown xenografts (45-69%, P<0.0001; FIG. 14F).

### LIVE Knockdown Exerts Broad Effects on the VEGF Axis

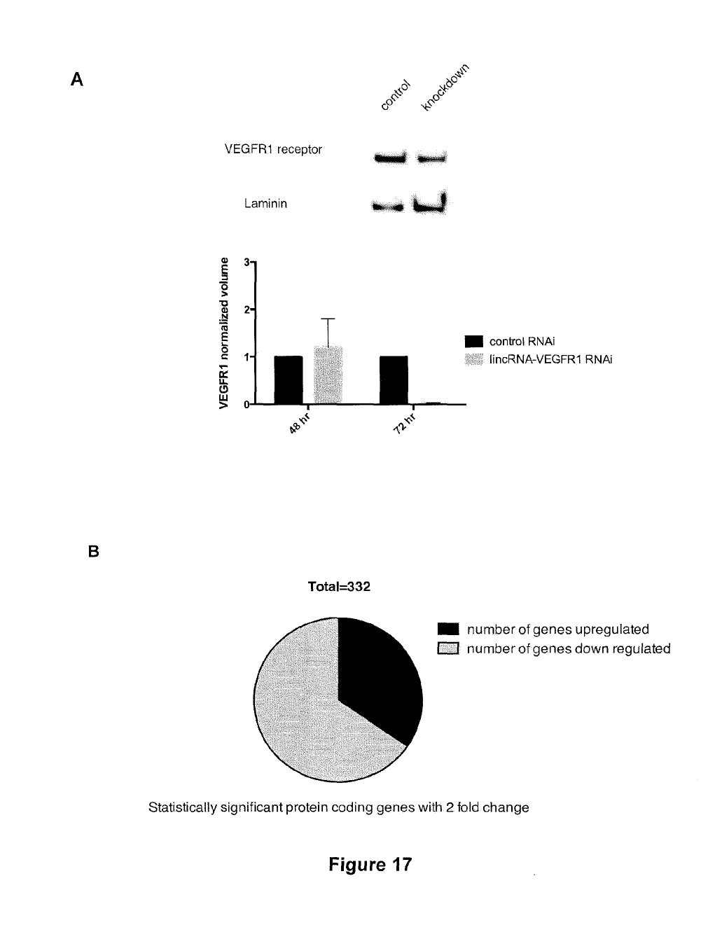

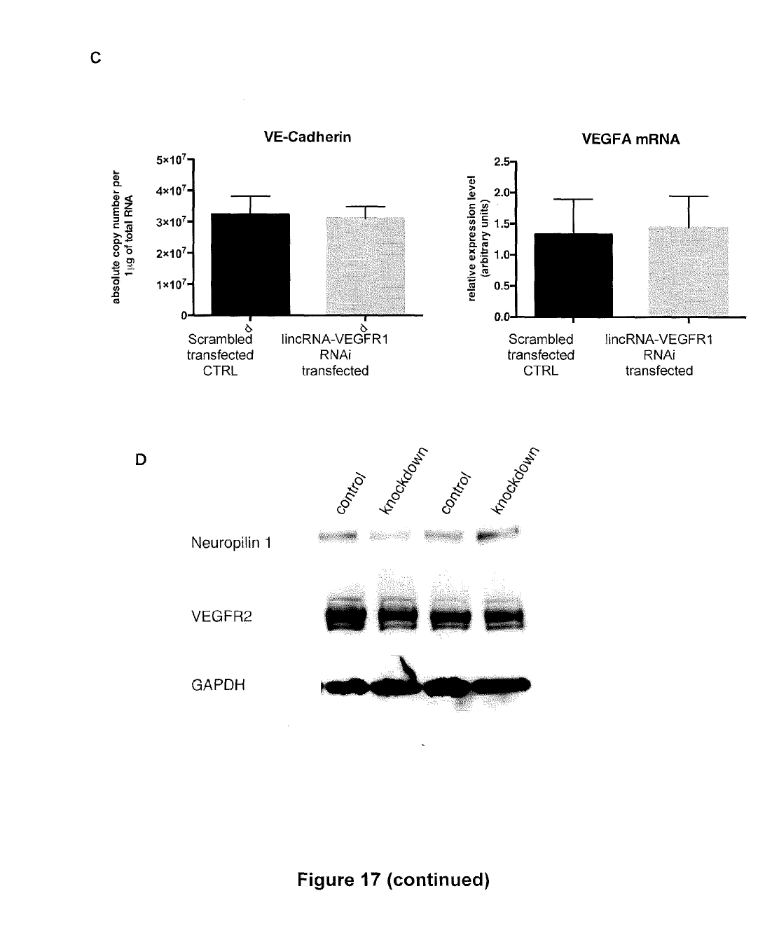

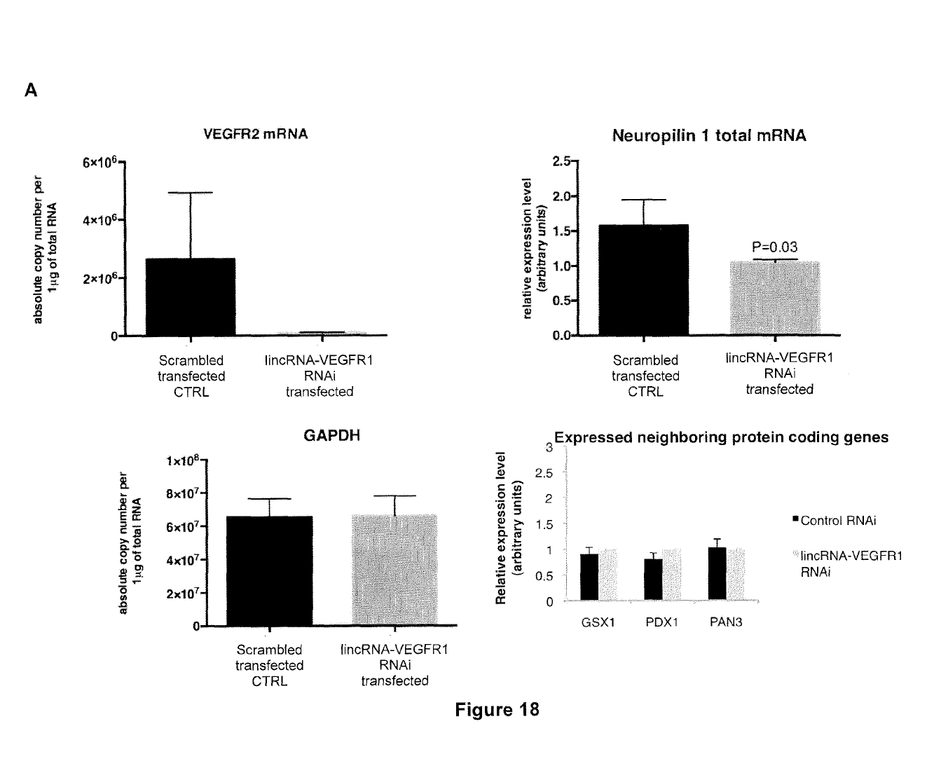

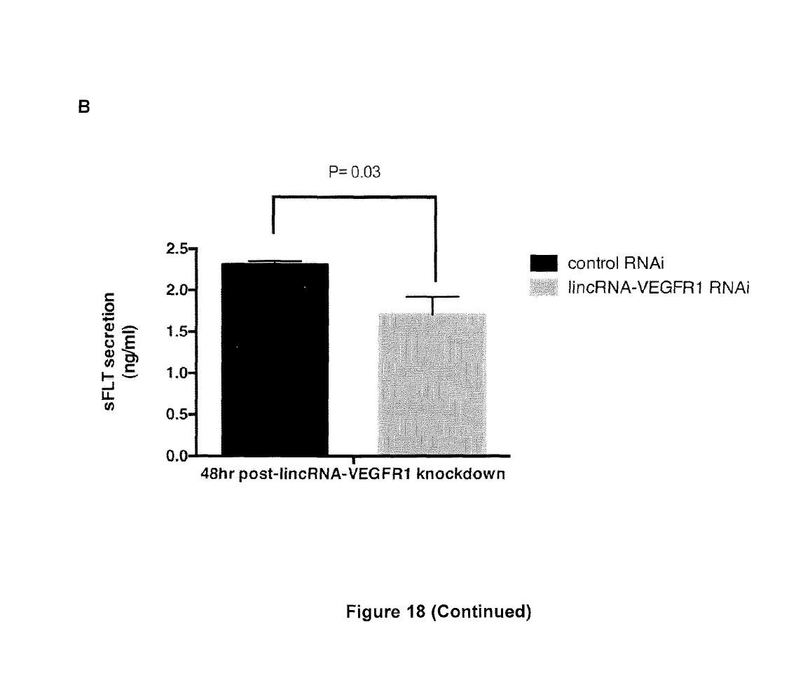

To assess the effect of LIVE knockdown on overall gene expression, microarray profiling was performed on four distinct biological samples of HUVECs transfected with either scrambled control or dsiRNA targeting LIVE (FIG. 17A). Two dsiRNAs against LIVE were used. Of the top statistically significant downregulated genes identified by the microarray, neuropilin 1 (NRP1), a VEGF receptor, was significantly downregulated by 34% and VEGFR2 transcript levels decreased over 10-fold, though the results from absolute quantification failed to reach statistical significance because of variation in transcript copy number across biological samples (FIG. 17A, Table 3). Other top candidates, such as VE-Cadherin, demonstrated no change when assayed by absolute quantification by qRT-PCR (FIG. 17C and Table 3). Moreover, neighboring genes within the genomic context of LINCRNAVEGFRI and VEGFR1 were unaffected by LIVE knockdown (FIG. 18A). To assess the impact of LIVE knockdown on de novo synthesized VEGFR1 protein products, ELISA of secreted VEGFR1 was used. Media collected from HUVECs grown in monolayers treated with either control RNAi or LIVE RNAi for 48 and 72 hr was assayed. LIVE RNAi treatment resulted in a significant decrease in secreted VEGFR1 (FIG. 17A). Decreases in stable membrane-bound VEGFR1 as determined by western blot were not significant (FIG. 17A).

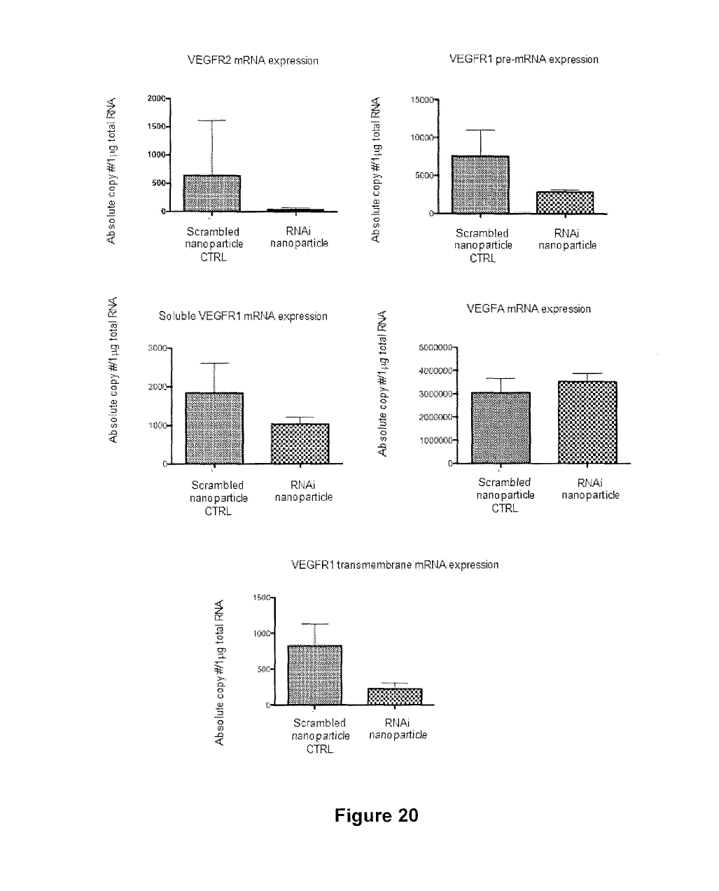

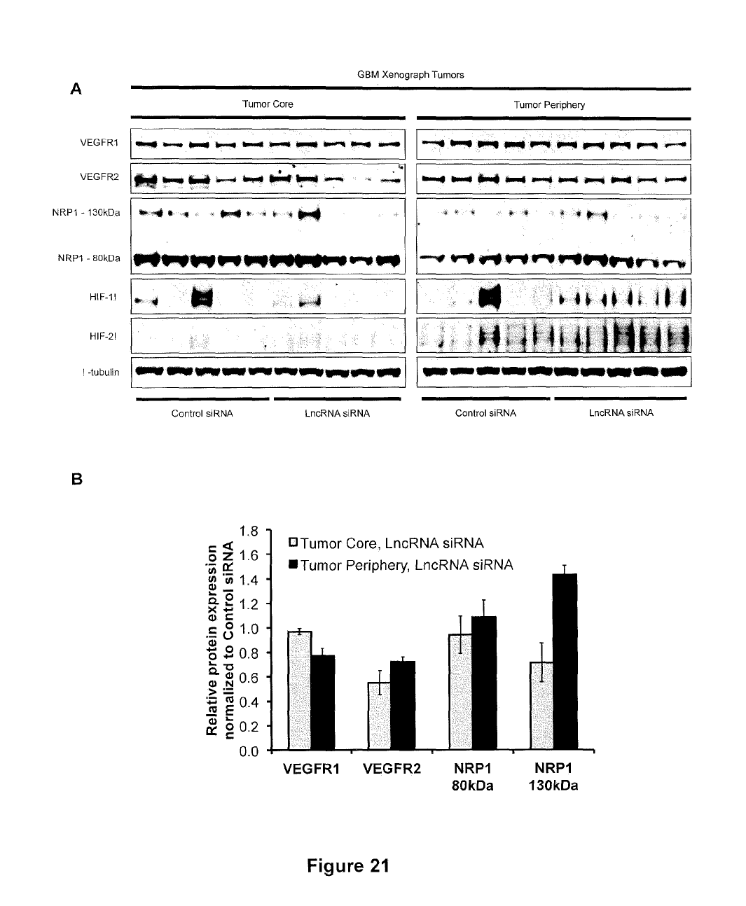

It was then asked if the cis location of LIVE was critical for induction of the neighboring VEGFR1 locus. To address this issue, a heterologous expression vector was designed for episomal expression of LIVE in HUVECs. Transcription, overall mature mRNA abundance and protein levels were assessed. Twenty-four hours post-transfection, LIVE over-expression led to a 3-fold increase in the transcription of endogenous VEGFR1 pre-mRNA as well as VEGFR1 trans-membrane receptor mature mRNA (FIG. 19A). At 48 hours post-transfection, although VEGFR1 pre-mRNA was consistently higher compared to control, mature mRNA transcripts of the VEGFR1 gene were expressed at levels comparable to control transfection (FIG. 19A). Western blot analysis for VEGFR1 protein demonstrated no difference between cells transfected with control or LIVE over-expression vector at 48 hours (FIG. 19B). In the heterotopic xenografts, VEGF-A mRNA levels were unchanged between the treatment and control groups, however, VEGFR1 and VEGFR2 transcriptional products decreased in abundance as determined by qRT-PCR (FIG. 20) (Seidel, Garvalov et al. 2010). Immunoblotting of core tumour extracts showed a decrease in VEGFR2 protein levels (FIG. 21).

Given its consistent effect on VEGFR1 transcription, a pGL2-hFIt1 (−1160 to +305) firefly luciferase promoter reporter was used for VEGFR1 to investigate whether LIVE acted on the VEGFR1 promoter. pGL2-eNOS(−1001 to +109) and pGL2-Basic (empty promoter reporter vector) were used as negative controls. pGL2-Control (SV40 promoter) was used as a positive control for firefly luciferase expression. All promoter reporter plasmids were co-transfected with either LIVE over-expression plasmid or empty pCDNA vector along with pRL-SV40 to normalize for transfection efficiency. LIVE expression vector led to an increase in relative luciferase activity only when co-transfected with pGL2-hFlt1 (−1160 to +305) compared to pCDNA control, indicating a VEGFR1 promoter-specific effect (FIG. 18B).

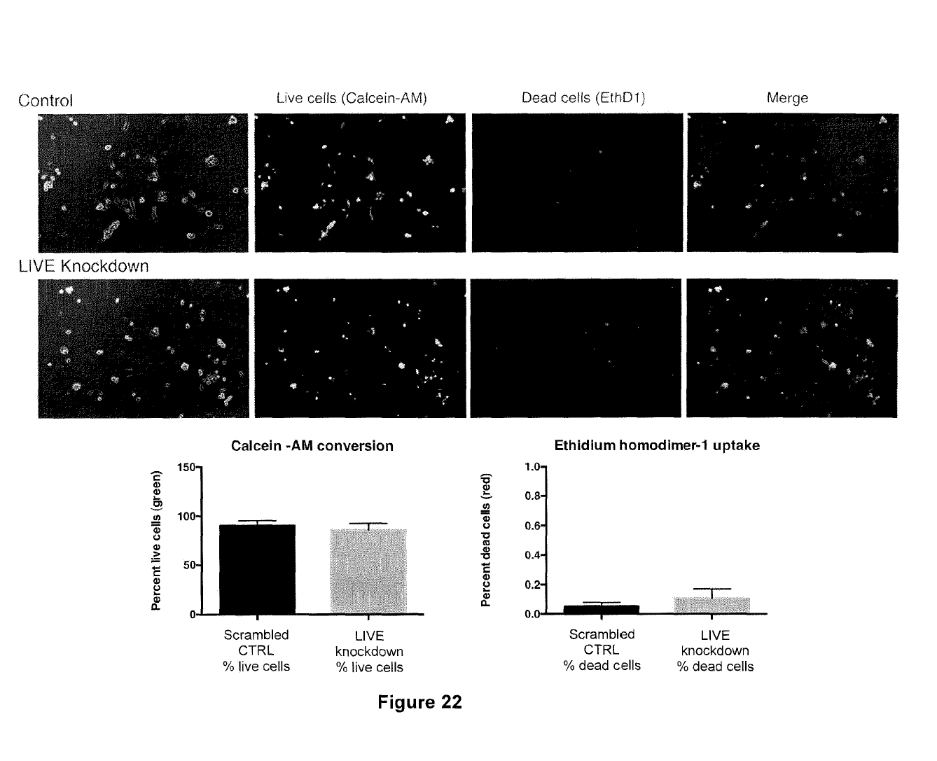

Experiments designed to investigate effects of LIVE depletion in glioma stem cell viability or vitality showed there were no cellular effects. Live/dead staining was performed on GliNS1 GSCs 72-hours post-transfection with either dsiRNA against LIVE or scrambled control (FIG. 22). No difference was observed between LIVE knockdown and scrambled control. Cy3-labeled dsiRNA was used to measure transfection efficiency (N=3, 100 μm; non-significant vs scrambled control).

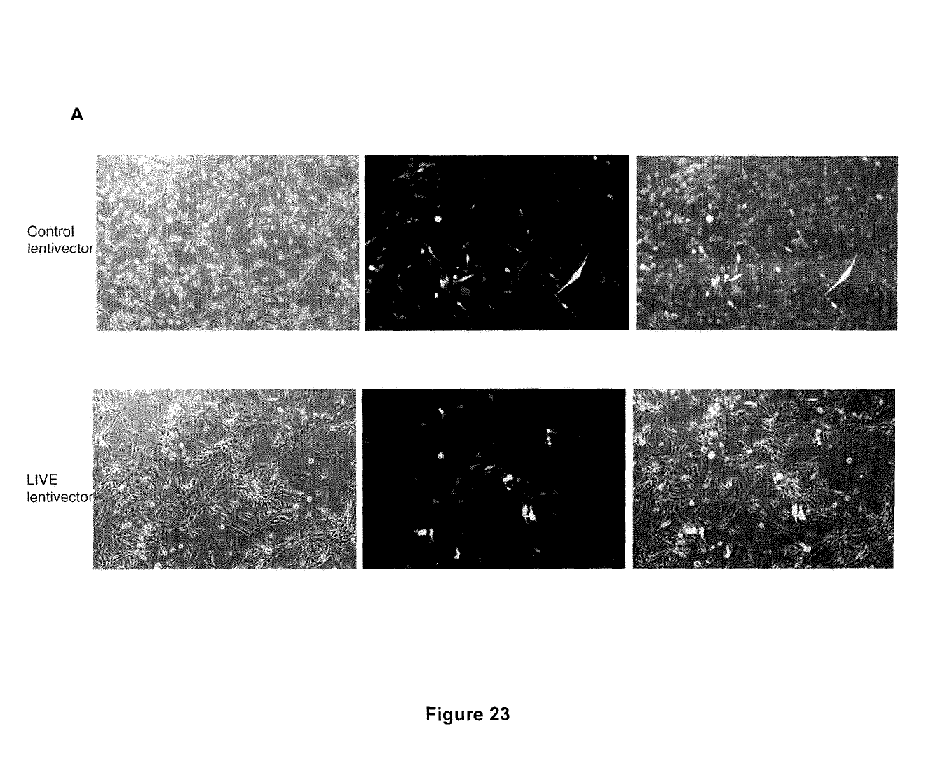

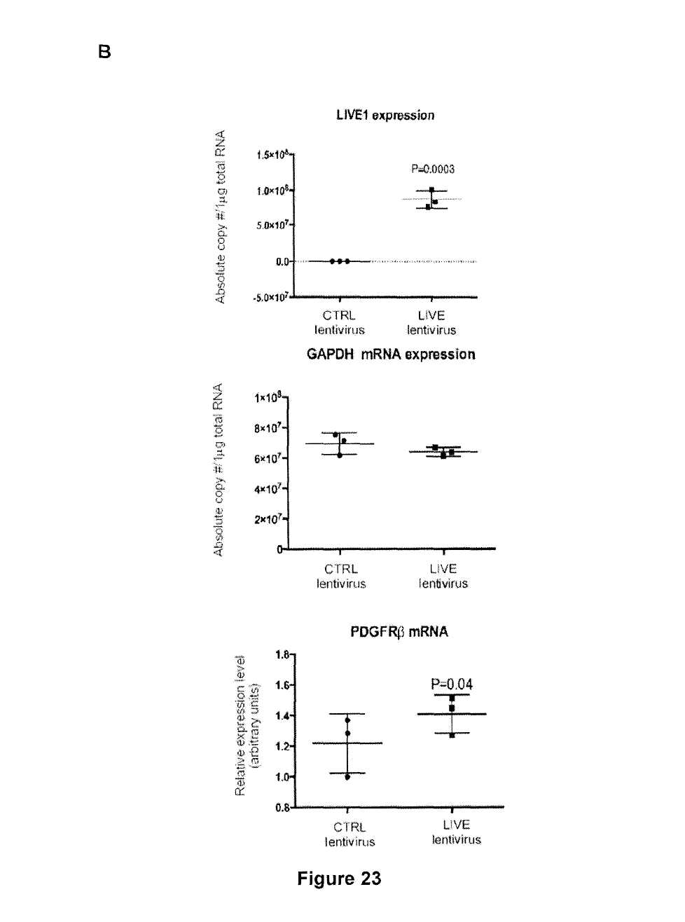

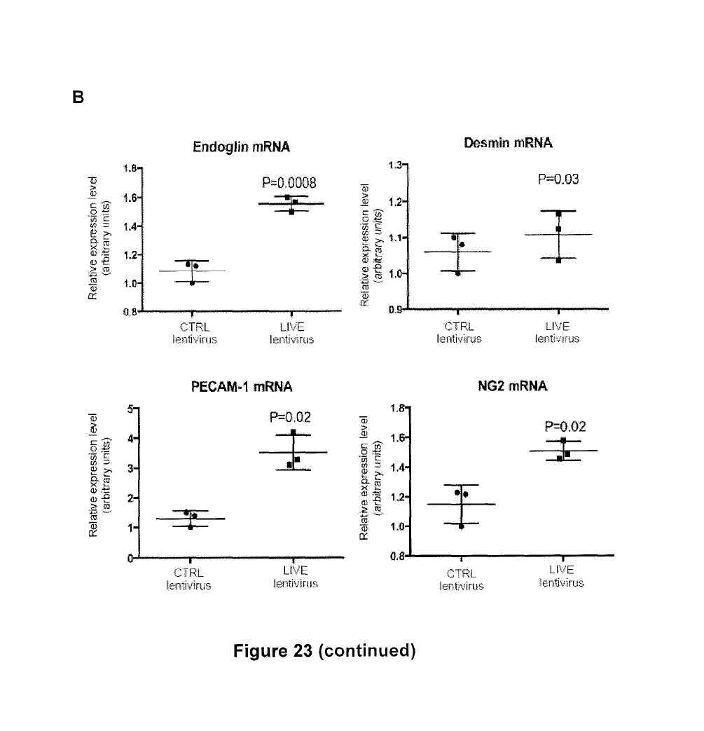

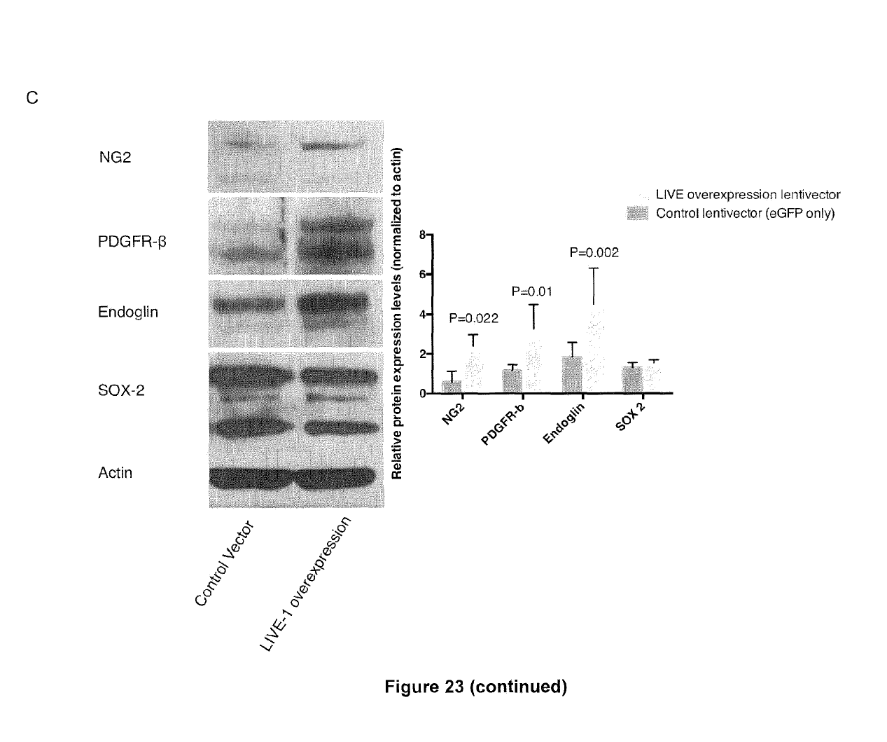

In low LIVE expressing glioma stem cells, LIVE over-expression led to significant increases in pericyte and endothelial marker expression at the RNA and protein levels (FIG. 23). Low LIVE expressing GSC line GSC2012035 was transduced with LIVE-containing lentivirus at a multiplicity of infection (MOI) of 2. Cells were visualized and collected at 72 hrs post-infection (N=4, scale bar=100 μm). RNA was collected and assayed for the expression of LIVE and GAPDH via absolute quantification with standard curve and luciferase normalization. PDGFR-B, NG2, Endoglin, PECAM-1 and desmin levels were measured via delta CT in Viia7 (Life Technologies; N=3, mean±SEM, P-values vs. control lentivector shown). Western blot analysis performed on protein lysates collected at 72 hours post-infection show significant changes in the protein levels of NG2, PDGFR-β and endoglin, however, Sox2 expression levels remained unchanged (P-values vs. control lentivector shown; N=3, mean±SEM, multiple t-test Holm-Sidak method with alpha=5%). All statistical significance was determined by one-tailed student t-test unless otherwise indicated.

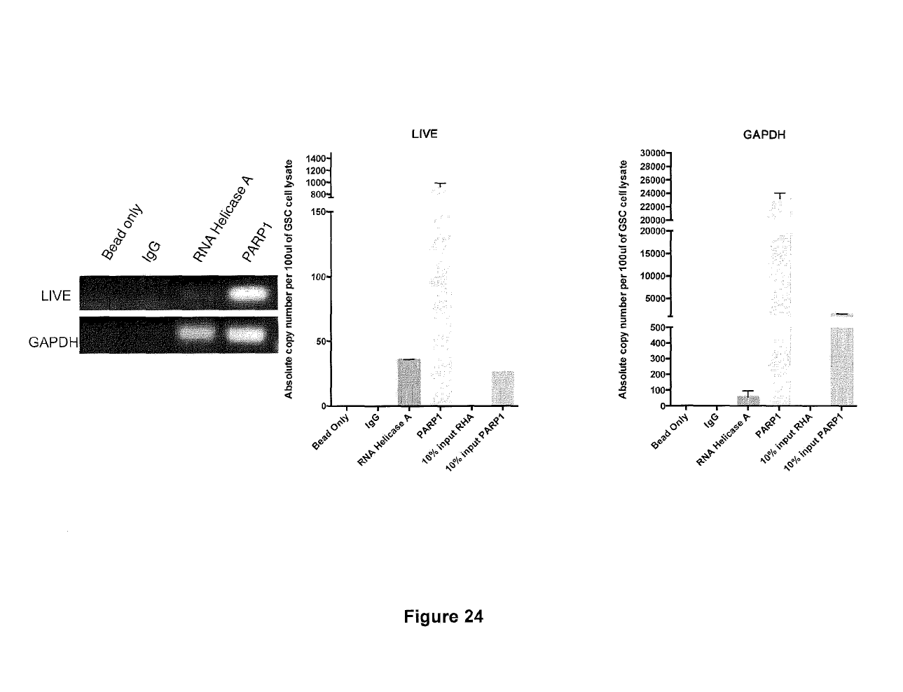

LIVE nuclear protein pull-down was coupled with mass spectrometry to identify protein-binding partners. LIVE was transcribed in vitro and used at two different concentrations for nuclear protein pull-down with GliNS1 nuclear lysates. Samples were sent for tandem mass spectrometry and results were analyzed with GO annotations. Various binding partners are shown in Table 2 including PARP1 and RHA which were the subject of further investigation. RNA-binding protein immunoprecipitation (RIP) was then used to confirm association of LIVE with RHA and PARP1 in GLiNS1 cell lysate (FIG. 24).

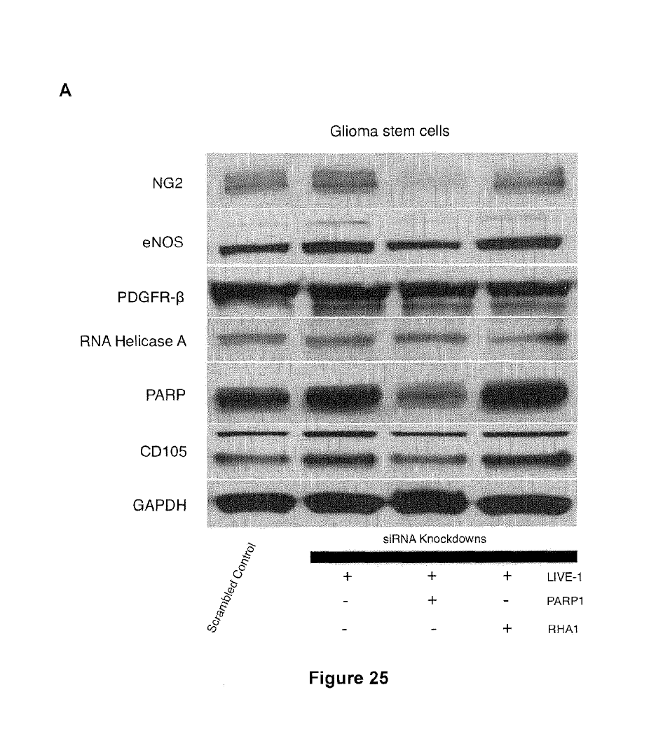

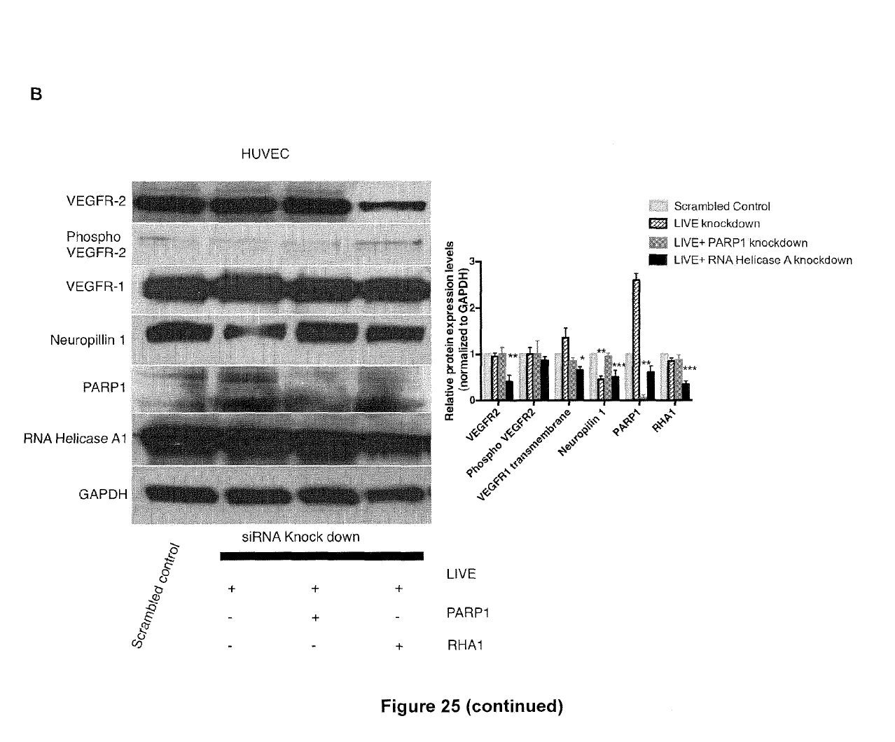

LIVE associates with PARP1 to drive endothelial and pericyte gene expression in glioma stem cells (FIG. 25). In human endothelial cells, LIVE associates with RHA to drive the expression of VEGF receptors. GliNS1s were subject to LIVE knockdown, LIVE+RHA knockdown using a single siRNA designed against/specific to RHA (SEQ ID NOs:17 and SEQ ID NO:18), LIVE+PARP1 knockdown using a single siRNA designed against and specific for PARP1 (SEQ ID NOs:15 and 16) or scrambled control (SEQ ID NOs:12 and 13). Western blots were performed 72 hrs after treatment (N=3; representative image). HUVECs were subject to knockdown with LIVE or double knockdown with LIVE+PARP1 or RNA helicase 1. Westerns blots were 72 hr post-transfection, significant decreases in VEGFR2, VEGFR1 and Neuropilin1 was observed (FIG. 17D)(Normalized to GAPDH, *=P<0.05, **=P<0.01, *=P<0.001, two-way ANOVA).

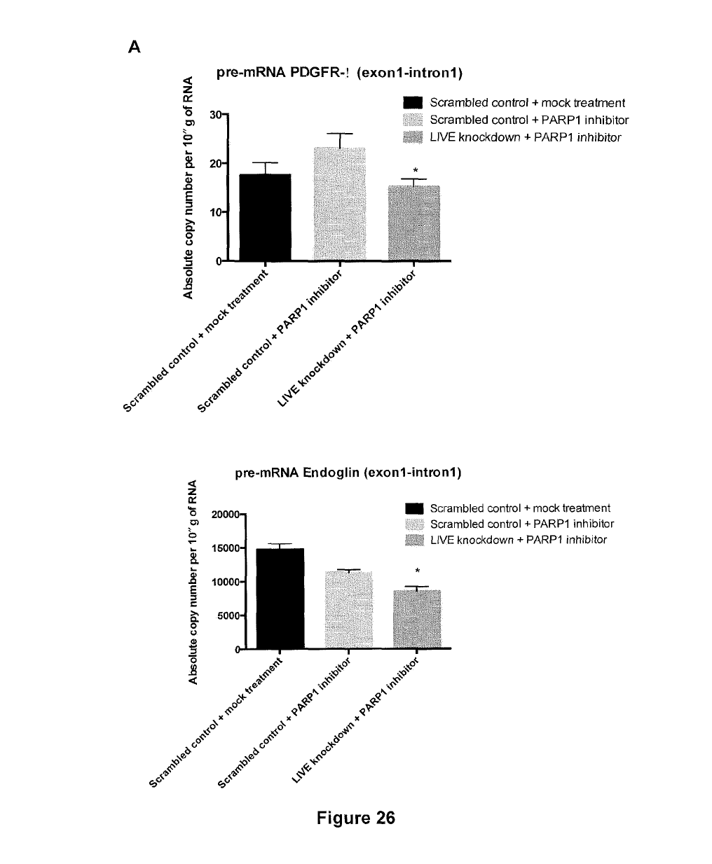

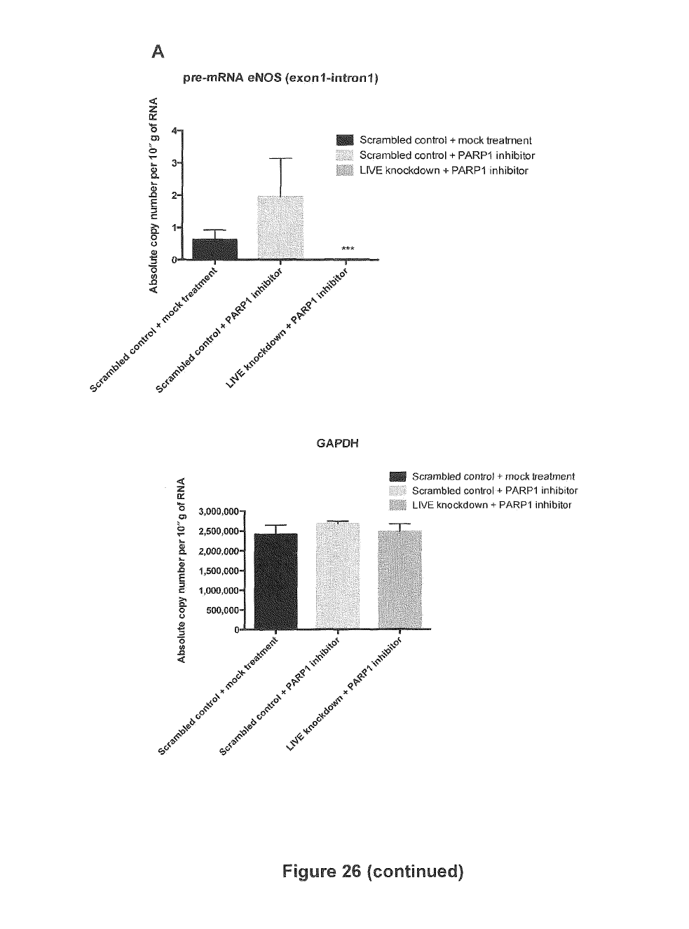

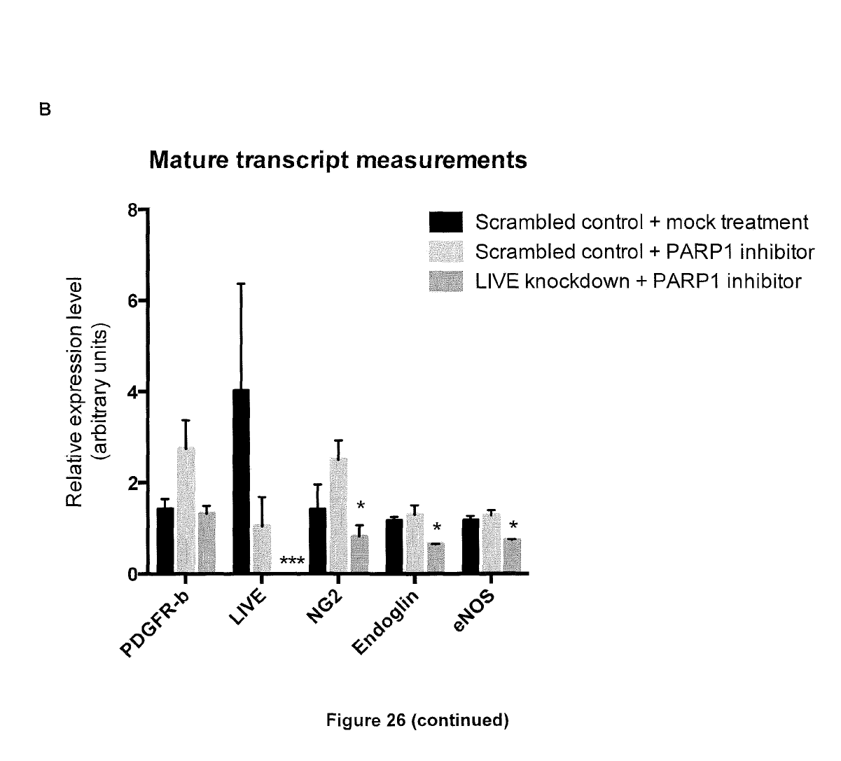

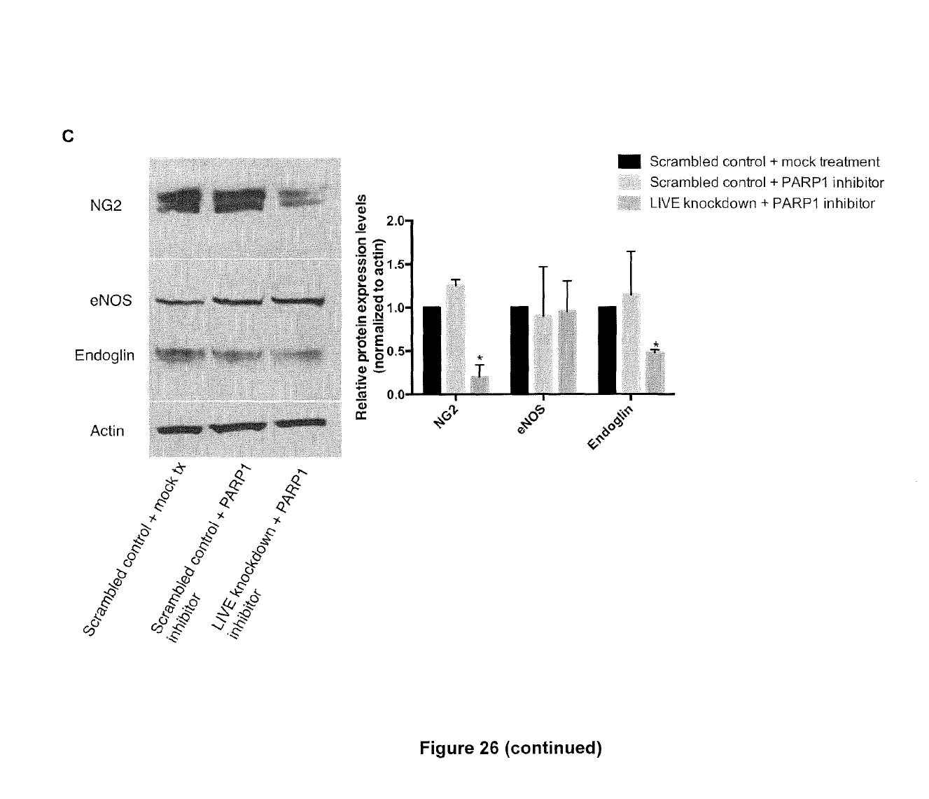

Combined with the PARP1-specific inhibitor, BYK204165, depletion of LIVE leads to decreases in endothelial and pericyte gene expression in glioma stem cells (FIG. 26). GliNS1s were subject to scrambled control, scrambled control with BYK204165 treatment or LIVE knockdown with BYK204165 treatment. RNA was reverse transcribed and pre-mRNA and mRNA targets were measured via qRT-PCR (N=3, mean±s.d.). (j) Western blots were performed on exact experimental conditions delineated above (N=3; representative image, normalized to actin, *=P<0.05, *=P<0.01, ***=P<0.001, two-way ANOVA).

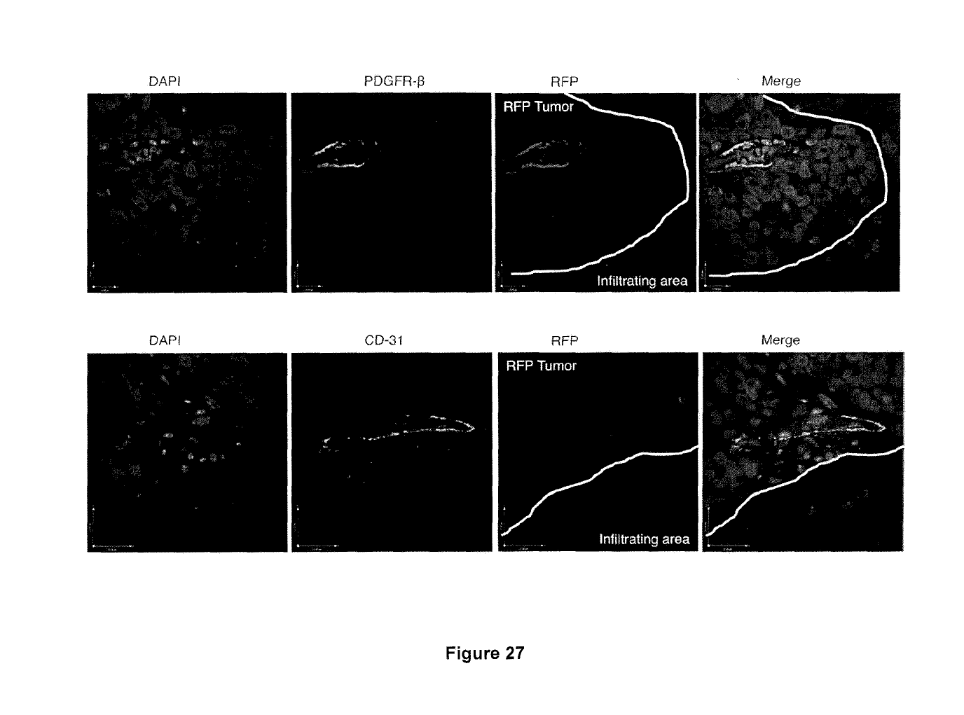

Glioma stem cells give rise to vascular pericytes in xenografts and not endothelial cells (FIG. 27). Co-immunofluorescence (Co-IF) staining of orthotopic intracranial xenografts derived from RFP reporter GliNS1s using PDGFR-β and CD-31 antibodies demonstrates RFP only co-localizes with PDGFR-β (representative image; N=4, scale bar=13 μm). Without wishing to be bound by theory, this further supports the role of LIVE in glioma stem cell-mediated angiogenesis in glioblastoma multiforme by offering a novel mechanism by which LIVE contributes to tumor vascularization by directing glioma stem cell differentiation into vascular pericytes to support tumor vasculature.

### DISCUSSION

The present inventors have demonstrated a novel lincRNA that functions as a VEGF-A responsive element and is relevant to angiogenesis and vascular formation in normal human endothelial cells and in glioblastoma. Previously, lncRNAs have been shown to enhance transcription of neighboring genes by acting as molecular scaffolds to recruit various proteins to the genomic region (Feng, Bi et al. 2006; Orom, Derrien et al. 2010; Wang, Yang et al. 2011). The majority of these lncRNAs can be categorized as lincRNAs and are not to be confused with recently discovered enhancer-templated non-coding RNA (eRNA) (Feng, Bi et al. 2006; Wang, Garcia-Bassets et al. 2011; Wang, Yang et al. 2011). LincRNAs such as HOTTIP need to be expressed in cis in a distance-dependent manner in order to exert their function, whereas LIVE can be expressed in an ectopic manner to exert transcriptional enhancement (Wang, Yang et al. 2011). In terms of overall function, LIVE is a VEGF-responsive cellular effector molecule that is required for vascular network formation. Given the complex nature of angiogenesis as well as the transcriptomic profile upon LIVE knockdown, it is clear that there is a global transcriptomic effect and VEGFR1 is not the only target of LIVE. Knockdown of LIVE consistently decreases transcriptional output of VEGFR1 gene, thus newly synthesized and secreted sFLT1 may be more reflective of de novo synthesis post-knockdown than overall cellular bound VEGFR1 due to mechanisms of protein stability.

As both normal endothelial cells and GSCs hold angiogenic potential, it is of interest that a lincRNA that has functional effects on vascular network formation would be expressed and enriched in both of these cell types. However, angiogenesis in a normal physiological context involves processes distinct from vascularization in a neoplastic state. Due to the fact that LIVE is enriched in the glioma neoplastic endothelial precursor (CD133⁺CD144⁺) population and its expression lost in the terminally differentiated CD133⁻CD144⁺ population, it is likely that pro-angiogenic LIVE expression in the endothelial precursor population is an important component of the angiogenic mechanism in glioblastoma. LIVE may also be expressed in other cell subpopulations in GBM, such as pericytes.

The in vivo tumour xenograft studies demonstrate the therapeutic potential of lincRNAs and recapitulate in vitro knockdown findings in both healthy human endothelial cells and endothelial-conditioned GSCs. Nanoparticle-mediated delivery of RNAi against LIVE decreased VEGFR1 trans-membrane signaling protein expression, microvessel density, perfused vascular counts and pericyte coverage in heterotopic xenograft tumours. Interestingly, HIFα expression and mRNAs known to be regulated by hypoxia were not significantly different in LIVE knockdown xenograft tumours versus control-treated tumours, suggesting that the differences in VEGFR1 expression and intratumoural vascularization between the RNAi- and control-treated group are not mediated by hypoxia (Dutta, Ray et al. 2008). A decrease in intratumoural microvasculature not only decreases the blood flow into the tumour, which in turn depletes the oxygen and nutrient supply within the tumour, but also decreases endothelial-glioma cell interactions. The decrease in tumour volume could also be due to the disruption of the perivascular niche, which is important for the maintenance of the GSC population (Calabrese, Poppleton et al. 2007; Hjelmeland, Lathia et al. 2011). Furthermore, intratumoural knockdown of LIVE decreases pericyte coverage, which could be contributing to the observed tumour regression. GSC-derived pericytes have recently been shown to be crucial for intratumoural vascularization and therapeutic targeting of GSC-derived pericytes have been shown to lead to tumor regression, which is consistent with the present findings (Cheng, Huang et al. 2013). In the present study, intratumoural knockdown of LIVE resulted in a decrease in perivascular cell proliferation and a decrease in Sox2-positive populations clustered around the microvasculature. The present examples affirm that lincRNAs are important cellular effector molecules that have significant therapeutic potential and merit attention as targets for pharmaceutical innovation.

While the present disclosure has been described with reference to what are presently considered to be the examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
(Table of Sequences)

| Primer Name | Sequence | SEQ ID NO: |
|---|---|---|
| 5′RACE specific | 5′cagcagcttcctcatccctgg3′ | 9 |
| primer 1 | | |
| Specific primer 2 | 5′tccccctctcactgaccatgg3′ | 10 |
| Specific primer 3 | 5′cctgagaggcctgtatggtgg3′ | 11 |
| siRNA sequence | Sequence | SEQ ID NO: |
| lineRNA-VEGFR1 DS | 5′ugaccauggcagagggagcagcucccu3′ | 4 |
| siRNA (1) | | |
| lineRNA-VEGFR1 DS | 3′acugguaccgucucccucgucgaggga5′ | 5 |
| siRNA (1) antisense | | |
| line-RNA-VEGFR1 | 5′ gagccuucgcacaagccuuuccugcug3′ | 6 |
| dsiRNA (II) | | |
| line-RNA-VEGFR1 | 3′ cucggaagcguguucggaaaggacgac5′ | 7 |
| dsiRNA (II) antisense | | |
| DS scrambled Neg | 5′cuuccucucuuucucucccuuguga3′ | 12 |
| Custom Scramble Neg | 5′cuuccucucuuucucucccuugccccc3′ | 13 |
| Cy3 DS control | 5′ Cy3-uccuuccucucuuucucucccuuguga3′ | 14 |
| PARP1 siRNA | 5′gcagcuucauaaccgaagatt3′ | 15 |
| (Silencer ™ Select; | | |
| life technologies) | | |
| Antisense | 5′ucuucgguuaugagcugctt3′ | 16 |
| RHA (DHX9; | 5′gguugaagcuuacuccggatt3′ | 17 |
| (Silencer ™ Select; | | |
| life technologies) | | |
| Antisense | 5′uccggaguaagcuucaaccac3′ | 18 |
| Cy3 DS control | 5′Cy3-tccuuccucucuuucucucccuuguga3′ | 19 |

LineRNA-VEGFR1 cDNA sequence
(SEQ ID NO: 1)
CTGGACCTTCCACAAGTGTTTGCACATGACCTGGAAGACAGTCAGAGGCAGAAGTCCCTGCAAATGCTC
CTGGCTACTTCCTGCAGGCAGCTCATGCTGCTGCAGCAGAAGACTTTCCTATGTCTAGCTGCCAGTATT
CTGGGTGCTGGGGGCATCCTGTTCCTAGGATCACAGCCATGGTGGTATGTTCTAGAACTCAGAAATTTA
CCAAATCACTGGATCGACCAGAGGAGTGAGCTCCAGGTTGGGTTTCCAGAAGGGACTCCCAGAACTCTT
CCACCATACAGGCCTCTCAGGGAGCTGCTCCCTCTGCCATGGTCAGTGAGAGGGGGAAGCAGGAGCCGC
CATTGGGGTTGTTGAGTTCGTGGCTGCAACCCAGGGATGAGGAAGCTGCTGCTACACACCCATGAAGCT
GATGCCTGGACATAAATCCCTACTGATAAGTGTTTACGACATTTCCAGCGTGGTGCCGACACTGCATGG
AAATGCTGCATGGAAAGTCCTTATACATCTATCTTTGTGCATTTATTGTGAGCACCTACTATGAGTAAA
ACCTGGGCTGGTGGCTGGAGAAACATGAAGATGAGTAAGAGCCAATTCCTGTTCTTGGGGATTTAATAA
TATATTCAAGGGAAAAGACACAAAATAACCATTTCCAGGTAAACTCTGGTGGGGAGGGTGGGAGGAGAA
CCGTGGTTTGCTTTTGTGCCCAACACTTCACATTCCTTACCTCTTTCTCCCCACCGAGACCTTGAGGAG
CAGCCTGAGCCAGAGGACCAGCATCCTCATCTTCTCTCCACCTCTTACTAGGGGGGTGGCTTTGGGCCA
GTTTCTTACATTCTTTGGGCCACTTTAGTTTCCTTATTGAAAAATGGGGATAATAATAGTGGCTACATC
TCAGTGTGATTCTGAGGAAACCAGAATTGTACATGCAGAGCACATGCACAGAACAGTGCCTGGCACAGT
CAGTAATTGATCAATGTGCGCTATTGTTGTTGTGTATATTAGTTCTCCCCTGATACAGATGAGAGCTAT
CTCTAACTCAGAGACTTGCCACATCATTAAATTAGGAGTGAGAACTGAGCCTGGATAAGGAAGAGGAAC
AGGAATTCAACACAGTGAACACAGCAGGAAAGGCTTGTGCGAAGGCTCTGGGCTGGAGATGTGTAAAGC
ATGTCTGGAGATGGGGAAGGTCCATTGGGCCAGGAAACCACAGGCTCTGCTCTCCTGGAGCAGAAGCAC
AGCGAAATCACAGCCGAGCAGAGGAGGGCAAGGGAGAGGGCCGCCCCAGTCTGAGAGCTGCAGGGTCTG
GCTGGAACTGCTCCCGGCCAGCGGACTTCACCCGGGCGCGGGGGCCGCACCTGCCGGGCGCGGCCTGCT
CTATGGCGCCCTCTGCTGTTAGTCCGCCCCAGGCTCCGCGCCGGCCTCTCCTGGGTCCGTGGGGCCTGC
GGGCTGCGGGGATCACCGAGACCCACATTCCCGTGGCCAGCAGCCTTTCGCTCTGCTCAGAGGAGAGGC
AGAAGGGCATATTGCTGTTTCCCAGTCGCTTTTTACACCTGCCTTCTTCGGATAAACCCAAAAATCTTC
CTTCAGAGAAGACGGCCCGTATTTCCCGTTATTTGGGGGTGGAGGTGGGGCTAAGGGCGTCATAGGGAG
AGCCTTACTTTCAACATTCTGCATTATGAAACCAAGGGAGACTTTTTTTCCCAACAAGTGTGAACATTT
TTTTTCAAGAGAATTAAATCGTTTAT
*Alternating unbold and bold indicates exons within the transcript
**Underlined sequences are the targets of dsiRNA knockdown
LineRNA-VEGFR1 mRNA sequence
(SEQ ID NO: 8)
CUGGACCUUCCACAAGUGUUUGCACAUGACCUGGAAGACAGUCAGAGGCAGAAGUCCCUGCAAAUGCUC
CUGGCUACUUCCUGCAGGCAGCUCAUGCUGCUGCAGCAGAAGACUUUCCUAUGUCUAGCUGCCAGUAUU
CUGGGUGCUGGGGGCAUCCUGUUCCUAGGAUCACAGCCAUGGUGGUAUGUUCUAGAACUCAGAAAUUUA
CCAAAUCACUGGAUGGACCAGAGGAGUGAGCUCCAGGUUGGGUUUCCAGAAGGGACUCCCAGAACUCUU
CCACCAUACAGGCCUCUCAGGGAGCUGCUCCCUCUGCCAUGGUCAGUGAGAGGGGGAAGCAGGAGCCGC
CAUUGGGGUUGUUGAGUUCUGUGGCUGCAACCCAGGGAUGAGGAAGCUGCUGCUACACACCCAUGAAGC
UGAUGCCUGGACAUAAAUCCCUACUGAUAAGUGUUUACGACAUUUCCAGCGUGGUGCCGACACUGCAUG
GAAAUGCUGCAUGGAAAGUCCUUAUACAUCUAUCUUUGUGCAUUUAUUGUGAGCACCUACUAUGAGUAA
AACCUGGGCUGGUGGCUGGAGAAACAUGAAGAUGAGUAAGAGCCAAUUCCUGUUCUUGGGGAUUUAAUA
AUAUAUUCAAGGGAAAAGACACAAAAUAACCAUUUCCAGGUAAACUCUGGUGGGGAGGGUGGGAGGAGA
ACCGUGGUUUGCUUUUGUGCCCAACACUUCACAUUCCUUACCUCUUUCUCCCCACCGAGACCUUGAGGA
GCAGCCUGAGCCAGAGGACCAGCAUCCUCAUCUUCUCUCCACCUCUUACUAGGGGGGUGGCUUUGGGCC
AGUUUCUUACAUUCUUUGGGCCACUUUAGUUUCCUUAUUGAAAAAUGGGGAUAAUAAUAGUGGCUACAU
CUCAGUGUGAUUCUGAGGAAACCAGAAUUGUACAUGCAGAGCACAUGCACAGAACAGUGCCUGGCACAG
UCAGUAAUUGAUCAAUGUGCGCUAUUGUUGUUGUGUAUAUUAGUUCUCCCCUGAUACAGAUGAGAGCUA
UCUCUAACUCAGAGACUUGCCACAUCAUUAAAUUAGGAGUGAGAACUGAGCCUGGAUAAGGAAGAGGAA
CAGGAAUUCAACACAGUGAACACAGCAGGAAAGGCUUGUGCGAAGGCUCUGGGCUGGAGAUGUGUAAAG
CAUGUCUGGAGAUGGGGAAGGUCCAUUGGGCCAGGAAACCACAGGCUCUGCUCUCCUGGAGCAGAAGCA
CAGCGAAAUCACAGCCGAGCAGAGGAGGGCAAGGGAGAGGGCCGCCCCAGUCUGAGAGCUGCAGGGUCU
GGCUGGAACUGCUCCCGGCCAGCGGACUUCACCCGGGCGCGGGGGCCGCACCUGCCGGGCGCGGCCUGC
UCUAUGGCGCCCUCUGCUGUUAGUCCGCCCCAGGCUCCGCGCCGGCCUCUCCUGGGUCCGUGGGGCCUG
CGGGCUGCGGGGAUCACCGAGACCCACAUUCCCGUGGCCAGCAGCCUUUCGCUCUGCUCAGAGGAGAGG
CAGAAGGGCAUAUUGCUGUUUCCCAGUCGCUUUUUACACCUGCCUUCUUCGGAUAAACCCAAAAAUCUU
CCUUCAGAGAAGACGGCCCGUAUUUCCCGUUAUUUGGGGGUGGAGGUGGGGCUAAGGGCGUCAUAGGGA
GAGCCUUACUUUCAACAUUCUGCAUUAUGAAACCAAGGGAGACUUUUUUUCCCAACAAGUGUGAACAUU
UUUUUUCAAGAGAAUUAAAUCGUUUAU
*Alternating unbold and bold indicates exons within the transcript
**Underlined sequences are the targets of dsiRNA knockdown

TABLE 2
LIVE binding partners identified via mass spectroscopy.

| Binding Partner | GenBank Accession No. |
|---|---|
| ATP-dependent RNA helicase A (DHX9) | sp\|Q08211\|DHX9_human |
| Poly(ADP-ribose) polymerase 1 (PARP1) | PARP1_human |
| DNA-dependent protein kinase catalytic subunit (PRKDC) | E7EUY0_human |
| Myosin-10 (MYH10) | F8VTL3_human |
| Pre-mRNA-processing-splicing factor 8 (PRPF8) | PRP8_human |
| Interleukin enhancer binding factor 3, isoform CRA_b (ILF3) | G5E9M5_human |
| Isoform beta-1 of DNA topoisomerase 2-beta (TOP2B) | sp\|Q02880-2\|TOP2B_human |
| Heterogeneous nuclear ribonucleoprotein R (HNRNPR) | Sp\|O43390\|HNRPR_human |
| Probable ATP-dependent RNA helicase DDX5 (DDX5) | DDX5_human |
| Heterogeneous nuclear ribonucleoprotein A1 (HNRNPA1) | sp\|P09651\|ROA1_human |
| U5 small nuclear ribonucleoprotein 200 kDa helicase (SNRNP200) | sp\|075643\|U520_human |
| Heterogeneous nuclear ribonucleoproteins A2 (HNRNPA2B1) | sp\|P22626\|ROA2_human |
| DNA topoisomerase 1 (TOP1) | TOP1_human |
| Heterogeneous nuclear ribonucleoprotein U (HNRNPU) | sp\|Q00839\|HNRPU_human |
| Nuclcolar RNA helicase 2 (DDX21) | sp\|Q9NR30\|DDX21_human |
| Interleukin enhancer-binding factor 2 (ILF2) | ILF2_human |

TABLE 3

| | P-Value | Fold change |
|---|---|---|
| Top Protein | | |
| coding genes | | |
| down regulated | | |
| upon knockdown | | |
| VEGFR2 (KDR) | 0.032286786 | 6.0603495 |
| Beta 1 laminin | 0.042480692 | 4.7357855 |
| Neuropilin 1 (NRP1) | 0.038766786 | 2.2537851 |
| Jagged 2 | 0.028555268 | 2.4257205 |
| Pim-1 | 0.047159757 | 2.8305585 |
| Protocadherin 18 | 0.004101617 | 3.0197878 |
| VE-Cadherin | 0.049039003 | 2.420578 |
| Top Protein | | |
| coding genes | | |
| up-regulated | | |
| upon knockdown | | |
| CLL5 (RANTES) | 0.0309014 | 14.414031 |
| TNF | 0.040116537 | 12.645307 |
| HSP70 | 0.019905597 | 5.650953 |
| GATA 5 | 0.000531443 | 3.522768 |
| HOXB9 | 0.013234486 | 2.409886 |
| KLF 16 | 0.008766258 | 2.7619383 |

### REFERENCES

Abe, M. and Y. Sato (2001). “cDNA microarray analysis of the gene expression profile of VEGF-activated human umbilical vein endothelial cells.” Angiogenesis 4(4): 289-298.
Arnaoutova, I. and H. K. Kleinman (2010). “In vitro angiogenesis: endothelial cell tube formation on gelled basement membrane extract.” Nat Protoc 5(4): 628-635.
Ashburner, M., C. A. Ball, et al. (2000). “Gene ontology: tool for the unification of biology. The Gene Ontology Consortium.” Nat Genet 25(1): 25-29.
Boeckel, J. N., V. Guarani, et al. (2011). “Jumonji domain-containing protein 6 (Jmjd6) is required for angiogenic sprouting and regulates splicing of VEGF-receptor 1.” Proc Natl Acad Sci USA 108(8): 3276-3281.
Cabili, M. N., C. Trapnell, et al. (2011). “Integrative annotation of human large intergenic noncoding RNAs reveals global properties and specific subclasses.” Genes Dev 25(18): 1915-1927.
Calabrese, C., H. Poppleton, et al. (2007). “A perivascular niche for brain tumor stem cells.” Cancer Cell 11(1): 69-82.
Cesana, M., D. Cacchiarelli, et al. (2011). “A long noncoding RNA controls muscle differentiation by functioning as a competing endogenous RNA.” Cell147(2): 358-369.
Chappell, J. C., S. M. Taylor, et al. (2009). “Local guidance of emerging vessel sprouts requires soluble Fit-1.” Dev Cell 17(3): 377-386.
Cheng, L., Z. Huang, et al. (2013). “Glioblastoma stem cells generate vascular pericytes to support vessel function and tumor growth.” Cell 153(1): 139-152.
Claffey, K. P., S. C. Shih, et al. (1998). “Identification of a human VPF/VEGF 3′ untranslated region mediating hypoxia-induced mRNA stability.” Mol Biol Cell 9(2): 469-481.
Dutta, D., S. Ray, et al. (2008). “Activation of the VEGFR1 chromatin domain: an angiogenic signal-ETS1/HIF-2alpha regulatory axis.” J Biol Chem 283(37): 25404-25413.
Feng, J., C. Bi, et al. (2006). “The Evf-2 noncoding RNA is transcribed from the Dlx-5/6 ultraconserved region and functions as a Dlx-2 transcriptional coactivator.” Genes Dev 20(11): 1470-1484.
Ferrara, N., H. P. Gerber, et al. (2003). “The biology of VEGF and its receptors.” Nat Med 9(6): 669-676.
Fischer, S., M. Clauss, et al. (1999). “Hypoxia induces permeability in brain microvessel endothelial cells via VEGF and NO.” Am J Physiol 276(4 Pt 1): C812-820.
Fish, J. E., C. C. Matouk, et al. (2007). “Hypoxia-inducible expression of a natural cis-antisense transcript inhibits endothelial nitric-oxide synthase.” J Biol Chem 282(21): 15652-15666.
Flowers, M. A., Y. Wang, et al. (1995). “Reciprocal regulation of endothelin-1 and endothelial constitutive NOS in proliferating endothelial cells.” Am J Physiol 269(6 Pt 2): H1988-1997.
Gong, C. and L. E. Maquat (2011). “lncRNAs transactivate STAU1-mediated mRNA decay by duplexing with 3′ UTRs via Alu elements.” Nature 470(7333): 284-288.
Gupta, R. A., N. Shah, et al. (2010). “Long non-coding RNA HOTAIR reprograms chromatin state to promote cancer metastasis.” Nature 464(7291): 1071-1076.
Guttman, M., J. Donaghey, et al. (2011). “lincRNAs act in the circuitry controlling pluripotency and differentiation.” Nature 477(7364): 295-300.
He, Y., S. K. Smith, et al. (1999). “Alternative splicing of vascular endothelial growth factor (VEGF)-R1 (FLT-1) pre-mRNA is important for the regulation of VEGF activity.” Mol Endocrinol 13(4): 537-545.
Hjelmeland, A. B., J. D. Lathia, et al. (2011). “Twisted tango: brain tumor neurovascular interactions.” Nat Neurosci 14(11): 1375-1381.
Ho, J. J., J. L. Metcalf, et al. (2012). “Functional importance of Dicer protein in the adaptive cellular response to hypoxia.” J Biol Chem 287(34): 29003-29020.
Jensen, M. M., J. T. Jorgensen, et al. (2008). “Tumor volume in subcutaneous mouse xenografts measured by microCT is more accurate and reproducible than determined by 18F-FDG-microPET or external caliper.” BMC Med Imaging 8: 16.
Jin, J., K. Sison, et al. (2012). “Soluble FLT1 Binds Lipid Microdomains in Podocytes to Control Cell Morphology and Glomerular Barrier Function.” Cell 151(2): 384-399.
Kino, T., D. E. Hurt, et al. (2010). “Noncoding RNA gas5 is a growth arrest- and starvation-associated repressor of the glucocorticoid receptor.” Sci Signal 3(107): ra8.
Korff, T. and H. G. Augustin (1999). “Tensional forces in fibrillar extracellular matrices control directional capillary sprouting.” J Cell Sci 112 (Pt 19): 3249-3258.
Kotake, Y., T. Nakagawa, et al. (2011). “Long non-coding RNA ANRIL is required for the PRC2 recruitment to and silencing of p15(INK4B) tumor suppressor gene.” Oncogene 30(16): 1956-1962.
Kummar, S., Chen, A., et al. (2012). “Advances in using PARP inhibitors to treat cancer.” BMC Medicine 10:25.
Lai, M. C., Z. Yang, et al. (2011). “Long non-coding RNA MALAT-1 overexpression predicts tumor recurrence of hepatocellular carcinoma after liver transplantation.” Med Oncol. 29(3):1810-1816.
Leimgruber, A., S. Ostermann, et al. (2006). “Perfusion and diffusion MRI of glioblastoma progression in a four-year prospective temozolomide clinical trial.” Int J Radiat Oncol Biol Phys 64(3): 869-875.
Li, K., Y. Blum, et al. (2010). “A noncoding antisense RNA in tie-1 locus regulates tie-1 function in vivo.” Blood 115(1): 133-139.
Marsden, P. A., K. T. Schappert, et al. (1992). “Molecular cloning and characterization of human endothelial nitric oxide synthase.” FEBS Lett 307(3): 287-293.
Orom, U. A., T. Derrien, et al. (2010). “Long noncoding RNAs with enhancer-like function in human cells.” Cell 143(1): 46-58.
Paez-Ribes, M., E. Allen, et al. (2009). “Antiangiogenic therapy elicits malignant progression of tumors to increased local invasion and distant metastasis.” Cancer Cell 15(3): 220-231.
Pandey, R. R., T. Mondal, et al. (2008). “Kcnq1ot1 antisense noncoding RNA mediates lineage-specific transcriptional silencing through chromatin-level regulation.” Mol Cell 32(2): 232-246.
Parsa, H., R. Upadhyay, et al. (2011). “Uncovering the behaviors of individual cells within a multicellular microvascular community.” Proc Natl Acad Sci USA 108(12): 5133-5138.
Pollard, S. M., K. Yoshikawa, et al. (2009). “Glioma stem cell lines expanded in adherent culture have tumor-specific phenotypes and are suitable for chemical and genetic screens.” Cell Stem Cell 4(6): 568-580.
Prensner, J. R., M. K. lyer, et al. (2011). “Transcriptome sequencing across a prostate cancer cohort identifies PCAT-1, an unannotated lincRNA implicated in disease progression.” Nat Biotechnol 29(8): 742-749.
Ren, S., Z. Peng, et al. (2012). “RNA-seq analysis of prostate cancer in the Chinese population identifies recurrent gene fusions, cancer-associated long noncoding RNAs and aberrant alternative splicings.” Cell Res.
Ricci-Vitiani, L, R. Pallini, et al. (2010). “Tumour vascularization via endothelial differentiation of glioblastoma stem-like cells.” Nature 468(7325): 824-828.
Robb, G. B., A. R. Carson, et al. (2004). “Post-transcriptional regulation of endothelial nitric-oxide synthase by an overlapping antisense mRNA transcript” J Biol Chem 279(36): 37982-37996.
Roberts, K A., V. E. Abraira, et al. (2012). “Mutation of ruble, a novel long non-coding RNA located upstream of bmp4, causes vestibular malformation in mice.” PLoS One 7(1): e29495.
Seidel, S., B. K. Garvalov, et al. (2010). “A hypoxic niche regulates glioblastoma stem cells through hypoxia inducible factor 2 alpha.” Brain 133(Pt 4): 983-995.
Singh, S. K., C. Hawkins, et al. (2004). “Identification of human brain tumour initiating cells.” Nature 432(7015): 396-401.
Soda, Y., T. Marumoto, et al. (2011). “Transdifferentiation of glioblastoma cells Into vascular endothelial cells.” Proc Natl Acad Sci USA 108(11): 4274-4280.
Spoerke, E. D., N. G. Murray, et al. (2005). “A bioactive titanium foam scaffold for bone repair.” Acta Biomater 1(5): 523-533.
Stupp, R., W. P. Mason, et al. (2005). “Radiotherapy plus concomitant and adjuvant temozolomide for glioblastoma.” N Eng J Med 352(10): 987-996.
Takeda, N., K. Maemura, et al. (2004). “Endothelial PAS domain protein 1 gene promotes angiogenesis through the transactivation of both vascular endothelial growth factor and its receptor, Flt-1.” Circ Res 95(2): 146-153.
Tripathi, V., J. D. Ellis, et al. (2010). “The nuclear-retained noncoding RNA MALAT1 regulates alternative splicing by modulating SR splicing factor phosphorylation.” Mol Cell 39(6): 925-938.
Ulitsky, I., A. Shkumatava, et al. (2011). “Conserved function of lincRNAs in vertebrate embryonic development despite rapid sequence evolution.” Cell 147(7): 1537-1550.
Wang, D., I. Garcia-Bassets, et al. (2011). “Reprogramming transcription by distinct classes of enhancers functionally defined by eRNA.” Nature474(7351): 390-394.
Wang, K. C., Y. W. Yang, et al. (2011). “A long noncoding RNA maintains active chromatin to coordinate homeotic gene expression.” Nature 472(7341): 120-124.
Wang, R., K. Chadalavada, et al. (2010). “Glioblastoma stem-like cells give rise to tumour endothelium.” Nature 468(7325): 829-833.
Wary, K. K., G. D. Thakker, et al. (2003). “Analysis of VEGF-responsive genes involved in the activation of endothelial cells.” Mol Cancer 2: 25.
White, F. C., S. M. Carroll, et al. (1995). “VEGF mRNA is reversibly stabilized by hypoxla and persistently stabilized in VEGF-overexpressing human tumor cell lines.” Growth Factors 12(4): 289-301.
Yang, C., X. Li, et al. (2012). “Long non-coding RNA UCA1 regulated cell cycle distribution via CREB through PI3-K dependent pathway in bladder carcinoma cells.” Gene 496(1): 8-16.
Zhang, W., Y. Chen, et al. (2012). “Variants on chromosome 9p21.3 correlated with ANRIL expression contribute to stroke risk and recurrence in a large prospective stroke population.” Stroke 43(1): 14-21.

1.-3. (canceled)

4. A method of promoting angiogenesis comprising administering an isolated nucleic acid encoding a large intergenic RNA (LIVE), wherein the nucleic acid comprises the nucleic acid sequence as shown in SEQ ID NO:1 or a variant thereof, a vector of comprising the nucleic acid or a host cell transformed with the vector to a cell or animal in need thereof.

5. The method of claim 4, for treating ischemic heart disease, peripheral vascular disease, cerebrovascular disease or preeclampsia.

6. An isolated nucleic acid molecule that silences the expression of the large intergenic RNA encoded by SEQ ID NO:1 (LIVE).

7. The isolated nucleic acid molecule of claim 6 that targets the sequence AGGGAGCUGCUCCCUCUGCCAUGGUCA (SEQ ID NO:2) or the sequence CAGCAGGAAAGGCUUGUGCGAAGGCUC (SEQ ID NO:3).

8. The isolated nucleic acid molecule of claim 6 or 7, wherein the nucleic acid molecule is an antisense oligonucleotide or an siRNA molecule.

9. (canceled)

10. (canceled)

11. The isolated siRNA molecule of claim 8, wherein the siRNA molecule is a double stranded siRNA molecule comprising the sense strand 5′UGACCAUGGCAGAGGGAGCAGCUCCCU3′ (SEQ ID NO:4) and the antisense strand 5′AGGGAGCUGCUCCCUCUGCCAUGGUCA3′ (SEQ ID NO:5) or comprising nucleic acids which are at least 75%, 80%, 85% 90% or 95% identical to SEQ ID NO: 4 or 5, wherein U can also be T.

12. The isolated siRNA molecule of claim 8, wherein the siRNA molecule is a double stranded siRNA molecule comprising the sense strand 5′ GAGCCUUCGCACAAGCCUUUCCUGCUG3′ (SEQ ID NO:6) and the antisense strand 5′CAGCAGGAAAGGCUUGUGCGAAGGCUC3′ (SEQ ID NO: 7) or comprising nucleic acids which are at least 75%, 80%, 85% 90% or 95% identical to SEQ ID NO: 6 or 7, wherein U can also be T.

13. The isolated nucleic acid molecule of claim 6, wherein the nucleic acid molecule is chemically modified to increase stability.

14. (canceled)

15. (canceled)

16. A pharmaceutical composition comprising the isolated nucleic acid molecule of claim 6, and further comprising a PARP1 inhibitor and/or an RHA inhibitor.

17. A method of inhibiting angiogenesis comprising administering an agent that inhibits the large intergenic RNA (LIVE) encoded by the nucleic acid sequence as shown in SEQ ID NO:1 to a cell or animal in need thereof.

18. (canceled)

19. The method of claim 17, wherein the subject has cancer, wherein the cancer is melanoma, renal cell carcinoma, breast carcinoma, colon carcinoma, lung cancer or choriocarcinoma.

20. The method of claim 17, wherein the subject has cancer, wherein the cancer is glioblastoma.

21. The method of claim 17, for treating diabetic retinopathy, diabetic nephropathy, proliferative retinopathy, proliferative renal disease and wet age-related macular degeneration.

22. The method of claim 17, wherein the agent that inhibits LIVE is a siRNA molecule or antisense oligonucleotide.

23. The method of claim 22, wherein the siRNA molecule or antisense oligonucleotide is chemically modified to increase stability.

24. (canceled)

25. (canceled)

26. The method of claim 22, wherein the siRNA molecule or antisense oligonucleotide targets the sequence AGGGAGCUGCUCCCUCUGCCAUGGUCA (SEQ ID NO:2) or targets the sequence CAGCAGGAAAGGCUUGUGCGAAGGCUC (SEQ ID NO:3).

27. (canceled)

28. The method of claim 22, wherein the siRNA molecule is a double stranded siRNA molecule comprising the sense strand 5′UGACCAUGGCAGAGGGAGCAGCUCCCU3′ (SEQ ID NO:4) and the antisense strand 5′AGGGAGCUGCUCCUCUGCCAUGGUCA3′ (SEQ ID NO:5) or comprising nucleic acids which are at least 75%, 80%, 85% 90% or 95% identical to SEQ ID NO: 4 or 5, wherein U can also be T.

29. The method of claim 22, wherein the siRNA molecule is a double stranded siRNA molecule comprising the sense strand 5′ GAGCCUUCGCACAAGCCUUUCCUGCUG3′ (SEQ ID NO:6) and the antisense strand 5′CAGCAGGAAAGGCUUGUGCGAAGGCUC3′ (SEQ ID NO: 7) or comprising nucleic acids which are at least 75%, 80%, 85% 90% or 95% identical to SEQ ID NO: 6 or 7, wherein U can also be T.

30. The method of claim 17, further comprising administering a PARP1 inhibitor and/or an RHA inhibitor.

31. (canceled)

32. (canceled)
